(19) United States
(12) Patent Application Publication
(10) Pub. No.: US 2026/0242340 A1
(43) Pub. Date: Aug. 20, 2026

(54) COMPOUNDS AND METHODS FOR TREATING PROTEIN AGGREGATION DISEASES

(71) Applicant: ACELOT, INC., South San Francisco, CA (US)

(72) Inventors: Ambuj K. SINGH, Santa Barbara, CA (US); Jacob B. Schwarz, San Ramon, CA (US)

(21) Appl. No.: 19/472,024

(22) PCT Filed: Apr. 12, 2024
(86) PCT No.: PCT/US2024/024481

§ 371 Date: Oct. 2, 2025

Related U.S. Application Data
(60) Provisional application No. 63/459,196, filed on Apr. 13, 2023.

(51) Int. Cl. C07D 217/04; A61K 31/4035; A61K 31/472; A61P 25/28; C07D 209/44

(52) U.S. Cl. CPC C07D 217/04; A61K 31/4035; A61K 31/472; A61P 25/28; C07D 209/44

(57) ABSTRACT

Described herein, inter alia, are compounds and methods for treating protein aggregation diseases.

### CROSS-REFERENCES TO RELATED APPLICATIONS

This application claims the benefit of U.S. Provisional Application No. 63/459,196, filed Apr. 13, 2023, which is incorporated herein by reference in its entirety and for all purposes.

### BACKGROUND

Toxic protein aggregates spur neuronal degeneration and are a pathologic hallmark of multiple neurodegenerative diseases, such as Alzheimer's disease, amyotrophic lateral sclerosis, Parkinson's disease, and frontotemporal dementia. These toxic aggregates can be present both inside and outside of the cell. Across each of these diseases, there are many protein species that can develop toxic aggregates, such as β amyloid, alpha synuclein, tau, and TDP-43. A large percentage of patients have toxic aggregates from greater than one of these toxic protein aggregate species in the brain upon postmortem autopsy. Patients are usually diagnosed well after a large amount of these toxic aggregates have built up in the brain. Current FDA approved therapies for protein aggregates, such as recently approved anti-β amyloid antibody therapies, fail to fully treat these protein aggregate diseases in several ways: they have poor cell penetrance, and they do not physically break up existing protein aggregates. Disclosed herein, inter alia, are solutions to these and other problems in the art.

### BRIEF SUMMARY

In an aspect is provided a compound, or a pharmaceutically acceptable salt thereof, having the formula:

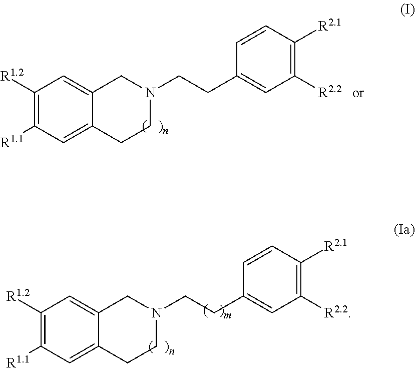

The symbol n is 0 or 1. The symbol m is an integer from 0 to 2.

R^{1.1} and R^{1.2} are independently hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, or substituted or unsubstituted heteroalkyl.

R^{2.1} and R^{2.2} are independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NR^{2C}C(O)NR^{2A}R^{2B}, —N(O)ₘ₂, —NR^{2A}R^{2B}, —C(O)R^{2C}, —C(O)OR^{2C}, —OC(O)R^{2C}, —OC(O)OR^{2C}, —C(O)NR^{2A}R^{2B}, —OC(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, or substituted or unsubstituted heteroalkyl.

R^{1A}, R^{1B}, R^{1C}, R^{1D}, R^{2A}, R^{2B}, R^{2C}, and R^{2D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl.

Each X¹ and X² is independently —F, —Cl, —Br, or —I. The symbols n1 and n2 are independently an integer from 0 to 4. The symbols m1, m2, v1, and v2 are independently 1 or 2.

In an aspect is provided a pharmaceutical composition including a compound described herein, or a pharmaceutically acceptable salt thereof, and a pharmaceutically acceptable excipient.

In an aspect is provided a method of treating a protein aggregation disease in a subject in need thereof, the method including administering to the subject a therapeutically effective amount of a compound described herein, or a pharmaceutically acceptable salt thereof.

In an aspect is provided a method of reducing formation of a protein aggregate or disrupting a protein aggregate, the method including contacting the protein aggregate with an effective amount of a compound described herein, or a pharmaceutically acceptable salt thereof.

### BRIEF DESCRIPTION OF THE DRAWINGS

FIGS. 1A and 1B are atomic force microscopy images of amyloid beta peptide 42 (Aβ42) protein aggregates and the effects of compound 1 thereon, as described in more detail in Example 1.

FIG. 2 shows three ion mobility mass spectrographs of TAR DNA-binding protein 43 (TDP-43), one with no protein aggregation disruption compound present (control), one with compound 1 present and one with compound 2 present, as more fully described in Example 2.

FIG. 3 shows two ion mobility mass spectrographs and two mass/charge figures for a TAR DNA-binding protein 43 (TDP-43) co-incubation experiment, one of each of the spectrographs and figures for no protein aggregation disruption compound present (controls) and one of each with compound 1 present. First row shows raw mass/charge ratio figures. Second row (the peak arrival times) shows protein size peaks.

FIG. 4 shows two ion mobility mass spectrographs and two mass/charge figures for a TAR DNA-binding protein 43 (TDP-43) co-incubation experiment, one of each of the spectrographs and figures for no protein aggregation disruption compound present (controls) and one of each with compound 2 present. First row shows raw mass/charge ratio figures. Second row (the peak arrival times) shows protein size peaks.

FIG. 5 shows two ion mobility mass spectrographs and two mass/charge figures for a TAR DNA-binding protein 43 (TDP-43) recovery experiment, one of each of the spectrographs and figures for no protein aggregation disruption compound present (controls) and one of each with compound 1 present. First row shows raw mass/charge ratio figures. Second row (the peak arrival times) shows protein size peaks.

FIG. 6 shows two ion mobility mass spectrographs and two mass/charge figures for a TAR DNA-binding protein 43 (TDP-43) recovery experiment, one of each of the spectrographs and figures for no protein aggregation disruption compound present (controls) and one of each with compound 2 present. First row shows raw mass/charge ratio figures. Second row (the peak arrival times) shows protein size peaks.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH—CH—O—CH₃, —Si(CH₃)₃, —CH₂—CH—N—OCH₃, —CH—CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzooxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

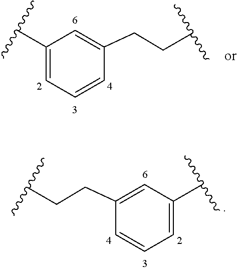 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NRC(NR′R″R′″)═NR″″, —NRC(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O) CH₃, —C(O) CF₃, —C(O) CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NR—C(NR′R″R′″)═NR″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur(S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R_{WW.1} or R^{LWW.1}, respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . . R^{100A.1}. R^{L1.1}, R^{L2.1}, R^{L3.1}, R^{L4.1}, R^{L5.1}, R^{L100.1}) may be further substituted with one or more second substituent groups (e.g., R^{1.2}, R^{2.2}, R^{3.2}, R^{4.2}, R^{5.2} . . . . R^{100.2}; R^{1A.2}, R^{2A.2}, R^{3A.2}, R^{4A.2}, R^{5A.2} . . . R^{100A.2}, R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

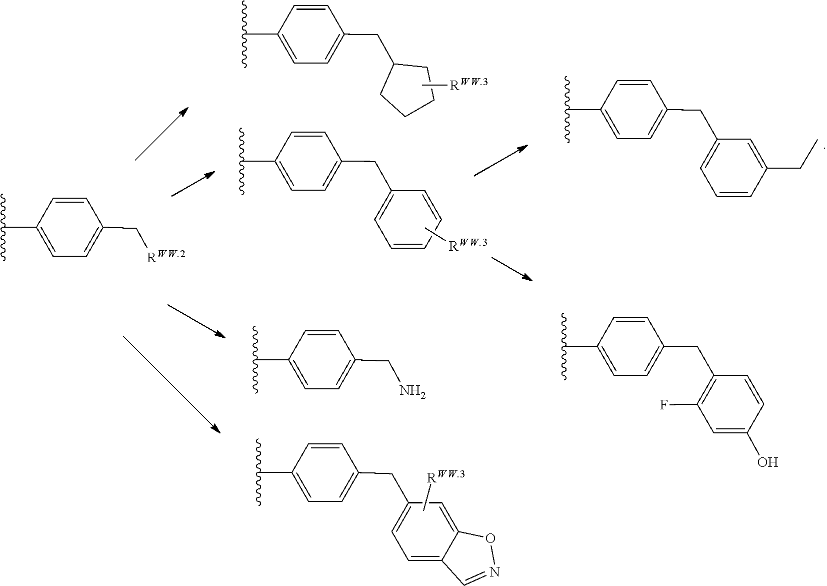

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}. R^{WW.2} is R^{100A.2}, and R^{WW.3} is R^{100A.3} Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}, R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O) NH—, —NHC(O)—, —NHC(O)NH—, —NHC(NH)NH—, —C(O)O—, —OC(O)—, —S—, —SO₂—, —SO₂NH—, R^{LWW.1}, substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or(S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and(S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently. Where an R moiety, group, or substituent as disclosed herein is attached through the representation of a single bond and the R moiety, group, or substituent is oxo, a person having ordinary skill in the art will immediately recognize that the oxo is attached through a double bond in accordance with the normal rules of chemical valency.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Co-administer” is meant that a composition described herein is administered at the same time, just prior to, or just after the administration of one or more additional therapies. The compounds disclosed herein can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease. In embodiments, treating is preventing. In embodiments, treating does not include preventing. In embodiments, the treating or treatment is no prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient”, “patient in need thereof”, “subject”, or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In embodiments, a patient is human. In embodiments, a patient in need thereof is human. In embodiments, a subject is human. In embodiments, a subject in need thereof is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is a protein aggregation disease. In embodiments, the disease is a neurodegenerative disease. In embodiments, the disease is diabetes. In embodiments, the disease is a p53 mutant cancer.

As used herein, the term “protein aggregation disease” refers to a disease or condition characterized by protein aggregation. In embodiments, the protein aggregation disease includes diseases outside the nervous system, such as primary systemic amyloidosis (AL amyloidosis), reactive systemic amyloidosis (AA amyloidosis), type II diabetes, injection-localized amyloidosis, beta-2 microglobulin amyloidosis, hereditary non-neuropathic amyloidosis, and Finnish hereditary systemic amyloidosis. In embodiments, the protein aggregation disease is a neurodegenerative disease. In embodiments, the protein aggregation disease is diabetes. In embodiments, the protein aggregation disease is a p53 mutant cancer.

As used herein, the term “neurodegenerative disease” refers to a disease or condition in which the function of a subject's nervous system becomes impaired. Examples of neurodegenerative diseases that may be treated with a compound, pharmaceutical composition, or method described herein include, but are not limited to, Alexander's disease, Alper's disease, Alzheimer's disease, amyloidosis, amyotrophic lateral sclerosis (ALS), amyotrophic lateral sclerosis and frontotemporal lobar degeneration, antibody light chain amyloidosis, Ataxia telangiectasia, Batten disease (also known as Spielmeyer-Vogt-Sjogren-Batten disease), bovine spongiform encephalopathy (BSE), C9orf72 amyotrophic lateral sclerosis, Canavan disease, Cockayne syndrome, corticobasal degeneration, Creutzfeldt-Jakob disease, dementia with Lewy bodies, diffuse Lewy body disease, facial onset sensory and motor neuropathy, familial amyloid polyneuropathy, frontotemporal dementia, frontotemporal lobar degeneration, frontotemporal lobar degeneration tau predominant pathology, frontotemporal lobar degeneration TDP-43 predominant pathology, genetic human prion disease, Gerstmann-Sträussler-Scheinker syndrome, hereditary cerebral amyloid angiopathy, Huntington's disease, HIV-associated dementia, Kennedy's disease, Krabbe's disease, kuru, Lewy body dementia, Machado-Joseph disease (spinocerebellar ataxia type 3), multiple sclerosis, multiple system atrophy, multiple system proteinopathy, myotonic disorder, narcolepsy, Neuroborreliosis, Parkinson's disease, Parkinson's disease-associated dementia, Pelizaeus-Merzbacher Disease, Perry syndrome, Pick's disease, poly-Q disease, primary lateral sclerosis, primary progressive aphasia, prion diseases, progressive supranuclear palsy, Refsum's disease, Sandhoff's disease, Schilder's disease, scrapie, semantic dementia, spinocerebellar ataxias and other Poly-Q diseases, sporadic amyotrophic lateral sclerosis, inclusion body myositis, subacute combined degeneration of spinal cord secondary to Pernicious Anaemia, schizophrenia, spinocerebellar ataxia (multiple types with varying characteristics), spinal muscular atrophy, spinocerebellar ataxia, Steele-Richardson-Olszewski disease, Tabes dorsalis, transthyretin amyloidosis, and variant Creutzfeldt-Jakob disease.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus medulloblastoma, colorectal cancer, or pancreatic cancer. Additional examples include Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

The term “p53 mutant cancer” as used herein refers to a cancer expressing a mutant p53 protein. p53 mutant cancers include, but are not limited to, small cell lung cancer, squamous cell lung cancer, triple-negative breast cancer, and high-grade serous ovarian cancer.

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorocarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, 198 Au, ¹⁹⁹Au, ²¹¹At, ²¹¹Pb, ²¹²Bi, ²¹²Pb, ²¹³Bi, ²²³Ra, and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

“Pharmaceutically acceptable excipient” and “pharmaceutically acceptable carrier” refer to a substance that aids the administration of an active agent to and absorption by a subject and can be included in the compositions disclosed herein without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds disclosed herein. One of skill in the art will recognize that other pharmaceutical excipients are useful in the presently disclosed pharmaceutical compositions.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies. The compounds disclosed herein can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions disclosed herein can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In therapeutic use for the treatment of a disease, compound(s) utilized in the pharmaceutical compositions disclosed herein may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., cancer) diagnosed in a particular patient. The dose administered to a patient, in the context of the presently disclosed methods of therapeutic treatment, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., cancer) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “electrophilic” as used herein refers to a chemical group that is capable of accepting electron density. An “electrophilic substituent,” “electrophilic chemical moiety,” or “electrophilic moiety” refers to an electron-poor chemical group, substituent, or moiety (monovalent chemical group), which may react with an electron-donating group, such as a nucleophile, by accepting an electron pair or electron density to form a bond.

“Nucleophilic” as used herein refers to a chemical group that is capable of donating electron density.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, Y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “protein aggregate” is used in accordance with its plain ordinary meaning and refers to an aberrant collection or accumulation of proteins (e.g., misfolded proteins). Protein aggregates are often associated with diseases (e.g., amyloidosis). In embodiments, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates (also referred to herein as amorphous protein aggregates), oligomers (also referred to herein as protein oligomers), and amyloid fibrils.

The terms “oligomer” and “protein oligomer” as used herein refer to a protein aggregate that has a size range of from a trimer to a 20-mer. In embodiments, the oligomer has a size range of from 40 kD to 500 kD.

The term “reducing formation of a protein aggregate” as used herein refers to decreasing formation of a protein aggregate relative to the absence of administration of a compound provided herein.

The term “disrupting a protein aggregate” as used herein refers to reducing or preventing formation of a protein aggregate relative to the absence of administration of a compound provided herein.

The term “amyloid beta” or “beta amyloid” is used in accordance with its plain ordinary meaning and refers to a mixture of peptides of 36 to 43 amino acids in length that are formed by sequential cleavage of the amyloid precursor protein (APP) by β- and γ-secretase. Amyloid beta is the main component of amyloid plaques found in the brains of people with Alzheimer's disease.

The term “amyloid beta peptide 42” or “Aβ42” or “Aβ 1-42” as used herein refers to a 42-amino acid isoform of amyloid beta.

The term “TAR DNA-binding protein 43” or “TDP-43” as used herein refers to a protein encoded by the TARDBP gene. In embodiments, TDP-43 has the amino acid sequence set forth in or corresponding to Entrez 23435, UniProt Q13148, or RefSeq (protein) NP 031401.1. In embodiments, the amino acid sequence is the sequence known at the time of filing of the present application.

The term “alpha-synuclein” or “aSyn” as used herein refers to a protein encoded by the SNCA gene. In embodiments, aSyn has the amino acid sequence set forth in or corresponding to Entrez 6622, UniProt P37840, RefSeq (protein) NP_000336.1, RefSeq (protein) NP_001139526.1, RefSeq (protein) NP_001139527.1, RefSeq (protein) NP_009292.1, or RefSeq (protein) NP_001362214. In embodiments, the amino acid sequence is the sequence known at the time of filing of the present application.

### II. Compounds

In an aspect is provided a compound, or a pharmaceutically acceptable salt thereof, having the formula:

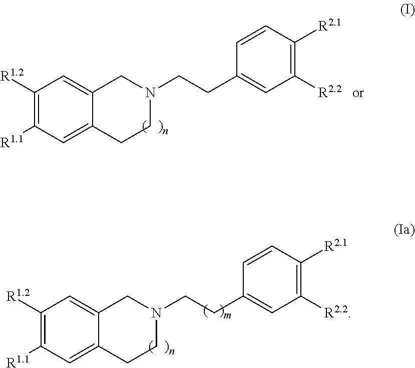

The symbol n is 0 or 1.

The symbol m is an integer from 0 to 2.

R^{1.1} and R^{1.2} are independently hydrogen, halogen, —CX¹₃, —CHX¹₂, —CH₂X¹, —OCX¹₃, —OCH₂X¹, —OCHX¹₂, —CN, —SO_{b1}R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

R^{2.1} and R^{2.2} are independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NR^{2C}C(O)NR^{2A}R^{2B}, —N(O)ₘ₂, —NR^{2A}R^{2B}, —C(O)R^{2C}, —C(O) OR^{2C}, —OC(O)R^{2C}, —OC(O)OR^{2C}, —C(O)NR^{2A}R^{2B}, —OC(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

R^{1A}, R^{1B}, R^{1C}, R^{1D}, R^{2A}, R^{2B}, R^{2C}, and R^{2D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

Each X¹ and X² is independently —F, —Cl, —Br, or —I.

The symbols n1 and n2 are independently an integer from 0 to 4.

The symbols m1, m2, v1, and v2 are independently 1 or 2.

In embodiments, the compound has the formula:

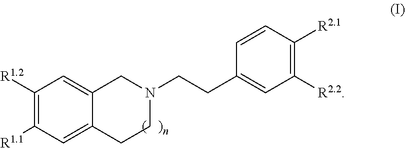

R^{1.1}, R^{1.2}, R^{2.1}, R^{2.2}, and n are as described herein, including in embodiments.

In an aspect is provided a compound, or a pharmaceutically acceptable salt thereof, having the formula:

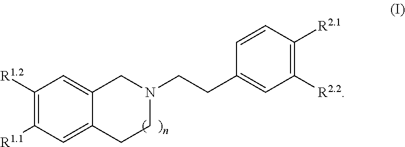

The symbol n is 0 or 1. R^{1.1} and R^{1.2} are independently hydrogen, halogen, —C(O)NR^{1A}R^{1B}, or —NR^{1A}C(O)R^{1C}. R^{2.1} and R^{2.2} are independently hydrogen, halogen, —CX²₃, —CHX²₂, —CH₂X², —OCX²₃, —OCH₂X², —OCHX²₂, —CN, —SOₙ₂R^{2D}, —SO₁₂NR^{2A}R^{2B}, —NR^{2C}NR^{2A}R^{2B}, —ONR^{2A}R^{2B}, —NR^{2C}C(O)NR^{2A}R^{2B}, —N(O)ₘ₂, —NR^{2A}R^{2B}, —C(O)R^{2C}, —C(O) OR^{2C}, —OC(O)R^{2C}, —OC(O)OR^{2C}, —C(O)NR^{2A}R^{2B}, —OC(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered). R^{1A}, R^{1B}, R^{1C}, R^{2A}, R^{2B}, R^{2C}, and R^{2D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered). Each X² is independently —F, —Cl, —Br, or —I. The symbol n2 is an integer from 0 to 4. The symbols m2 and v2 are independently 1 or 2. In embodiments, at least one of R^{1.1} and R^{1.2} is not hydrogen.

In an aspect is provided a compound, or a pharmaceutically acceptable salt thereof, having the formula:

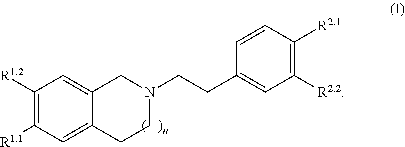

The symbol n is 0 or 1. R^{1.1} and R^{1.2} are independently hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O) OR^{1C}, —OC(O)R^{1C}, —OC(O) OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered). R^{2.1} and R^{2.2} are independently hydrogen or halogen. R^{1A}, R^{1B}, R^{1C}, and R^{1D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered). Each X¹ is independently —F, —Cl, —Br, or —I. The symbol n1 is an integer from 0 to 4. The symbols m1 and v1 are independently 1 or 2. In embodiments, at least one of R^{2.1} and R^{2.2} is not hydrogen.

In embodiments, the compound is not:

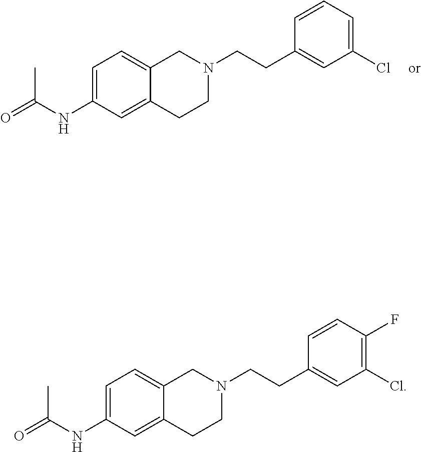 [SMILES: CC(=O)Nc1ccc2c(c1)CCN(CCc1cccc(Cl)c1)C2 ; CC(=O)Nc1ccc2c(c1)CCN(CCc1ccc(F)c(Cl)c1)C2]

In embodiments, each substituted alkyl or substituted heteroalkyl is not substituted with a substituent group that includes a cyclic group, e.g., cycloalkyl, heterocycloalkyl, aryl, or heteroaryl.

In embodiments, n is 0. In embodiments, n is 1.

In embodiments, a substituted R^{1.1} (e.g., substituted alkyl and/or substituted heteroalkyl) is substituted with at least one substituent group, size-limited substituent group, or lower substituent group; wherein if the substituted R^{1.1} is substituted with a plurality of groups selected from substituent groups, size-limited substituent groups, and lower substituent groups; each substituent group, size-limited substituent group, and/or lower substituent group may optionally be different. In embodiments, when R^{1.1} is substituted, it is substituted with at least one substituent group. In embodiments, when R^{1.1} is substituted, it is substituted with at least one size-limited substituent group. In embodiments, when R^{1.1} is substituted, it is substituted with at least one lower substituent group.

In embodiments, R^{1.1} is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, or substituted or unsubstituted heteroalkyl.

In embodiments, R^{1.1} is hydrogen or —OR^{1D}, wherein R^{1D} is as described herein, including in embodiments. In embodiments, R^{1.1} is hydrogen or —OR^{1D}, wherein R^{1D} is hydrogen or unsubstituted C₁-C₄ alkyl. In embodiments, R^{1.1} is hydrogen or —OCH₃.

In embodiments, R^{1.1} is hydrogen. In embodiments, R^{1.1} is halogen. In embodiments, R^{1.1} is —F. In embodiments, R^{1.1} is —Cl. In embodiments, R^{1.1} is —Br. In embodiments, R^{1.1} is —I. In embodiments, R^{1.1} is —CCl₃. In embodiments, R^{1.1} is —CBr₃. In embodiments, R^{1.1} is —CF₃. In embodiments, R^{1.1} is —Cl₃. In embodiments, R^{1.1} is —CH₂Cl. In embodiments, R^{1.1} is —CH₂Br. In embodiments, R^{1.1} is —CH₂F. In embodiments, R^{1.1} is —CH₂I. In embodiments, R^{1.1} is —CHCl₂. In embodiments, R^{1.1} is —CHBr₂. In embodiments, R^{1.1} is —CHF₂. In embodiments, R^{1.1} is —CHI₂. In embodiments, R^{1.1} is —CN. In embodiments, R^{1.1} is —OH. In embodiments, R^{1.1} is —NH₂. In embodiments, R^{1.1} is —COOH. In embodiments, R^{1.1} is —CONH₂. In embodiments, R^{1.1} is —NO₂. In embodiments, R^{1.1} is —SH. In embodiments, R^{1.1} is —SO₃H. In embodiments, R^{1.1} is —OSO₃H. In embodiments, R^{1.1} is —SO₂NH₂. In embodiments, R^{1.1} is —NHNH₂. In embodiments, R^{1.1} is —ONH₂. In embodiments, R^{1.1} is —NHC(O)NH₂. In embodiments, R^{1.1} is —NHSO₂H. In embodiments, R^{1.1} is —NHC(O)H. In embodiments, R^{1.1} is —NHC(O)OH. In embodiments, R^{1.1} is —NHOH. In embodiments, R^{1.1} is —OCCl₃. In embodiments, R^{1.1} is —OCBr₃. In embodiments, R^{1.1} is —OCF₃. In embodiments, R^{1.1} is —OCI₃. In embodiments, R^{1.1} is —OCH₂Cl. In embodiments, R^{1.1} is —OCH₂Br. In embodiments, R^{1.1} is —OCH₂F. In embodiments, R^{1.1} is —OCH₂I. In embodiments, R^{1.1} is —OCHCl₂. In embodiments, R^{1.1} is —OCHBr₂. In embodiments, R^{1.1} is —OCHF₂. In embodiments, R^{1.1} is —OCHI₂. In embodiments, R^{1.1} is —SF₅. In embodiments, R^{1.1} is —N₃. In embodiments, R^{1.1} is unsubstituted C₁-C₄ alkyl. In embodiments, R^{1.1} is unsubstituted methyl. In embodiments, R^{1.1} is unsubstituted ethyl. In embodiments, R^{1.1} is unsubstituted propyl. In embodiments, R^{1.1} is unsubstituted n-propyl. In embodiments, R^{1.1} is unsubstituted isopropyl. In embodiments, R^{1.1} is unsubstituted butyl. In embodiments, R^{1.1} is unsubstituted n-butyl. In embodiments, R^{1.1} is unsubstituted isobutyl. In embodiments, R^{1.1} is unsubstituted tert-butyl. In embodiments, R^{1.1} is unsubstituted 2 to 6 membered heteroalkyl. In embodiments, R^{1.1} is unsubstituted methoxy. In embodiments, R^{1.1} is unsubstituted ethoxy. In embodiments, R^{1.1} is unsubstituted propoxy. In embodiments, R^{1.1} is unsubstituted n-propoxy. In embodiments, R^{1.1} is unsubstituted isopropoxy. In embodiments, R^{1.1} is unsubstituted butoxy.

In embodiments, a substituted R^{1.2} (e.g., substituted alkyl and/or substituted heteroalkyl) is substituted with at least one substituent group, size-limited substituent group, or lower substituent group; wherein if the substituted R^{1.2} is substituted with a plurality of groups selected from substituent groups, size-limited substituent groups, and lower substituent groups; each substituent group, size-limited substituent group, and/or lower substituent group may optionally be different. In embodiments, when R¹² is substituted, it is substituted with at least one substituent group. In embodiments, when R^{1.2} is substituted, it is substituted with at least one size-limited substituent group. In embodiments, when R^{1.2} is substituted, it is substituted with at least one lower substituent group.

In embodiments, R^{1.2} is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, or substituted or unsubstituted heteroalkyl.

In embodiments, R^{1.2} is hydrogen or —OR^{1D}, wherein R^{1D} is as described herein, including in embodiments. In embodiments, R^{1.2} is hydrogen or —OR^{1D}, wherein R^{1D} is hydrogen or unsubstituted C₁-C₄ alkyl. In embodiments, R^{1.2} is hydrogen or —OCH₃.

In embodiments, R^{1.2} is hydrogen. In embodiments, R^{1.2} is halogen. In embodiments, R^{1.2} is —F. In embodiments, R^{1.2} is —Cl. In embodiments, R^{1.2} is —Br. In embodiments, R^{1.2} is —I. In embodiments, R^{1.2} is —CCl₃. In embodiments, R^{1.2} is —CBr₃. In embodiments, R^{1.2} is —CF₃. In embodiments, R^{1.2} is —Cl₃. In embodiments, R^{1.2} is —CH₂Cl. In embodiments, R^{1.2} is —CH₂Br. In embodiments, R^{1.2} is —CH₂F. In embodiments, R^{1.2} is —CH₂I. In embodiments, R^{1.2} is —CHCl₂. In embodiments, R^{1.2} is —CHBr₂. In embodiments, R^{1.2} is —CHF₂. In embodiments, R^{1.2} is —CHI₂. In embodiments, R^{1.2} is —CN. In embodiments, R^{1.2} is —OH. In embodiments, R^{1.2} is —NH₂. In embodiments, R^{1.2} is —COOH. In embodiments, R^{1.2} is —CONH₂. In embodiments, R^{1.2} is —NO₂. In embodiments, R^{1.2} is —SH. In embodiments, R^{1.2} is —SO₃H. In embodiments, R^{1.2} is —OSO₃H. In embodiments, R^{1.2} is —SO₂NH₂. In embodiments, R^{1.2} is —NHNH₂. In embodiments, R^{1.2} is —ONH₂. In embodiments, R^{1.2} is —NHC(O)NH₂. In embodiments, R^{1.2} is —NHSO₂H. In embodiments, R^{1.2} is —NHC(O)H. In embodiments, R^{1.2} is —NHC(O)OH. In embodiments, R^{1.2} is —NHOH. In embodiments, R^{1.2} is —OCCl₃. In embodiments, R^{1.2} is —OCBr₃. In embodiments, R^{1.2} is —OCF₃. In embodiments, R^{1.2} is —OCI₃. In embodiments, R^{1.2} is —OCH₂Cl. In embodiments, R^{1.2} is —OCH₂Br. In embodiments, R^{1.2} is —OCH₂F. In embodiments, R^{1.2} is —OCH₂I. In embodiments, R^{1.2} is —OCHCl₂. In embodiments, R^{1.2} is —OCHBr₂. In embodiments, R^{1.2} is —OCHF₂. In embodiments, R^{1.2} is —OCHI₂. In embodiments, R^{1.2} is —SF₅. In embodiments, R^{1.2} is —N₃. In embodiments, R^{1.2} is unsubstituted C₁-C₄ alkyl. In embodiments, R^{1.2} is unsubstituted methyl. In embodiments, R^{1.2} is unsubstituted ethyl. In embodiments, R^{1.2} is unsubstituted propyl. In embodiments, R^{1.2} is unsubstituted n-propyl. In embodiments, R^{1.2} is unsubstituted isopropyl. In embodiments, R^{1.2} is unsubstituted butyl. In embodiments, R^{1.2} is unsubstituted n-butyl. In embodiments, R^{1.2} is unsubstituted isobutyl. In embodiments, R^{1.2} is unsubstituted tert-butyl. In embodiments, R^{1.2} is unsubstituted 2 to 6 membered heteroalkyl. In embodiments, R^{1.2} is unsubstituted methoxy. In embodiments, R^{1.2} is unsubstituted ethoxy. In embodiments, R^{1.2} is unsubstituted propoxy. In embodiments, R^{1.2} is unsubstituted n-propoxy. In embodiments, R^{1.2} is unsubstituted isopropoxy. In embodiments, R^{1.2} is unsubstituted butoxy.

In embodiments, R^{1.1} is halogen, —C(O)NR^{1A}R^{1B}, or —NR^{1A}C(O)R^{1C}; and R^{1.2} is hydrogen. In embodiments, R^{1.1} is —NHC(O) CH₃; and R^{1.2} is hydrogen. In embodiments, R^{1.1} is —NCH₃C(O)CH₃; and R^{1.2} is hydrogen. In embodiments, R^{1.1} is —C(O)NHCH₃; and R^{1.2} is hydrogen. In embodiments, R^{1.1} is hydrogen; and R^{1.2} is halogen, —C(O)NR^{1A}R^{1B}, or —NR^{1A}C(O)R^{1C}. In embodiments, R^{1.1} is hydrogen; and R^{1.2} is —NHC(O) CH₃. In embodiments, R^{1.1} is hydrogen; and R^{1.2} is —NCH₃C(O)CH₃.

In embodiments, a substituted R^{1A} (e.g., substituted alkyl and/or substituted heteroalkyl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when R^{1A} is substituted, it is substituted with at least one size-limited substituent group. In embodiments, when RIA is substituted, it is substituted with at least one lower substituent group.

In embodiments, a substituted R^{1B} (e.g., substituted alkyl and/or substituted heteroalkyl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted R^{1C} (e.g., substituted alkyl and/or substituted heteroalkyl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, a substituted R^{1D} (e.g., substituted alkyl and/or substituted heteroalkyl) is substituted with at least one substituent group, size-limited substituent group, or lower substituent group; wherein if the substituted R^{1D} is substituted with a plurality of groups selected from substituent groups, size-limited substituent groups, and lower substituent groups; each substituent group, size-limited substituent group, and/or lower substituent group may optionally be different. In embodiments, when R^{1D} is substituted, it is substituted with at least one substituent group. In embodiments, when R^{1D} is substituted, it is substituted with at least one size-limited substituent group. In embodiments, when R^{1D} is substituted, it is substituted with at least one lower substituent group.

In embodiments, RIA is independently hydrogen. In embodiments, RIA is independently unsubstituted C₁-C₄ alkyl. In embodiments, R^{1A} is independently unsubstituted methyl. In embodiments, R^{1A} is independently unsubstituted ethyl. In embodiments, R^{1A} is independently unsubstituted propyl. In embodiments, R^{1A} is independently unsubstituted n-propyl. In embodiments, R^{1A} is independently unsubstituted isopropyl. In embodiments, R^{1A} is independently unsubstituted butyl. In embodiments, RIA is independently unsubstituted n-butyl. In embodiments, R^{1A} is independently unsubstituted isobutyl. In embodiments, R^{1A} is independently unsubstituted tert-butyl.

In embodiments, R^{1B} is independently hydrogen. In embodiments, R^{1B} is independently unsubstituted C₁-C₄ alkyl. In embodiments, R^{1B} is independently unsubstituted methyl. In embodiments, RIB is independently unsubstituted ethyl. In embodiments, R^{1B} is independently unsubstituted propyl. In embodiments, R^{1B} is independently unsubstituted n-propyl. In embodiments, R^{1B} is independently unsubstituted isopropyl. In embodiments, R^{1B} is independently unsubstituted butyl. In embodiments, R^{1B} is independently unsubstituted n-butyl. In embodiments, R^{1B} is independently unsubstituted isobutyl. In embodiments, R^{1B} is independently unsubstituted tert-butyl.

In embodiments, R^{1C} is independently hydrogen. In embodiments, R^{1C} is independently unsubstituted C₁-C₄ alkyl. In embodiments, R^{1C} is independently unsubstituted methyl. In embodiments, R^{1C} is independently unsubstituted ethyl. In embodiments, R^{1C} is independently unsubstituted propyl. In embodiments, R^{1C} is independently unsubstituted n-propyl. In embodiments, R^{1C} is independently unsubstituted isopropyl. In embodiments, R^{1C} is independently unsubstituted butyl. In embodiments, R^{1C} is independently unsubstituted n-butyl. In embodiments, R^{1C} is independently unsubstituted isobutyl. In embodiments, R^{1C} is independently unsubstituted tert-butyl.

In embodiments, R^{1D} is independently hydrogen. In embodiments, R^{1D} is independently unsubstituted C₁-C₄ alkyl. In embodiments, R^{1D} is independently unsubstituted methyl. In embodiments, R^{1D} is independently unsubstituted ethyl. In embodiments, R^{1D} is independently unsubstituted propyl. In embodiments, R^{1D} is independently unsubstituted n-propyl. In embodiments, R^{1D} is independently unsubstituted isopropyl. In embodiments, R^{1D} is independently unsubstituted butyl. In embodiments, R^{1D} is independently unsubstituted n-butyl. In embodiments, R^{1D} is independently unsubstituted isobutyl. In embodiments, R^{1D} is independently unsubstituted tert-butyl.

In embodiments, a substituted R^{2.1} (e.g., substituted alkyl and/or substituted heteroalkyl) is substituted with at least one substituent group, size-limited substituent group, or lower substituent group; wherein if the substituted R^{2.1} is substituted with a plurality of groups selected from substituent groups, size-limited substituent groups, and lower substituent groups; each substituent group, size-limited substituent group, and/or lower substituent group may optionally be different. In embodiments, when R^{2.1} is substituted, it is substituted with at least one substituent group. In embodiments, when R^{2.1} is substituted, it is substituted with at least one size-limited substituent group. In embodiments, when R^{2.1} is substituted, it is substituted with at least one lower substituent group.

In embodiments, R^{2.1} is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, or substituted or unsubstituted heteroalkyl.

In embodiments, R^{2.1} is hydrogen or halogen. In embodiments, R^{2.1} is hydrogen, —Cl, or —F.

In embodiments, R^{2.1} is hydrogen. In embodiments, R^{2.1} is halogen. In embodiments, R^{2.1} is —F. In embodiments, R^{2.1} is —Cl. In embodiments, R^{2.1} is —Br. In embodiments, R^{2.1} is —I. In embodiments, R^{2.1} is —CCl₃. In embodiments, R^{2.1} is —CBr₃. In embodiments, R^{2.1} is —CF₃. In embodiments, R^{2.1} is —Cl₃. In embodiments, R^{2.1} is —CH₂Cl. In embodiments, R^{2.1} is —CH₂Br. In embodiments, R^{2.1} is —CH₂F. In embodiments, R^{2.1} is —CH₂I. In embodiments, R^{2.1} is —CHCl₂. In embodiments, R^{2.1} is —CHBr₂. In embodiments, R^{2.1} is —CHF₂. In embodiments, R^{2.1} is —CHI₂. In embodiments, R^{2.1} is —CN. In embodiments, R^{2.1} is —OH. In embodiments, R^{2.1} is —NH₂. In embodiments, R^{2.1} is —COOH. In embodiments, R^{2.1} is —CONH₂. In embodiments, R^{2.1} is —NO₂. In embodiments, R^{2.1} is —SH. In embodiments, R^{2.1} is —SO₃H. In embodiments, R^{2.1} is —OSO₃H. In embodiments, R^{2.1} is —SO₂NH₂. In embodiments, R^{2.1} is —NHNH₂. In embodiments, R^{2.1} is —ONH₂. In embodiments, R^{2.1} is —NHC(O)NH₂. In embodiments, R^{2.1} is —NHSO₂H. In embodiments, R^{2.1} is —NHC(O)H. In embodiments, R^{2.1} is —NHC(O)OH. In embodiments, R^{2.1} is —NHOH. In embodiments, R^{2.1} is —OCCl₃. In embodiments, R^{2.1} is —OCBr₃. In embodiments, R^{2.1} is —OCF₃. In embodiments, R^{2.1} is —OCI₃. In embodiments, R^{2.1} is —OCH₂Cl. In embodiments, R^{2.1} is —OCH₂Br. In embodiments, R^{2.1} is —OCH₂F. In embodiments, R^{2.1} is —OCH₂I. In embodiments, R^{2.1} is —OCHCl₂. In embodiments, R^{2.1} is —OCHBr₂. In embodiments, R^{2.1} is —OCHF₂. In embodiments, R^{2.1} is —OCHI₂. In embodiments, R^{2.1} is —SF₅. In embodiments, R^{2.1} is —N₃. In embodiments, R^{2.1} is unsubstituted C₁-C₄ alkyl. In embodiments, R^{2.1} is unsubstituted methyl. In embodiments, R^{2.1} is unsubstituted ethyl. In embodiments, R^{2.1} is unsubstituted propyl. In embodiments, R^{2.1} is unsubstituted n-propyl. In embodiments, R^{2.1} is unsubstituted isopropyl. In embodiments, R^{2.1} is unsubstituted butyl. In embodiments, R^{2.1} is unsubstituted n-butyl. In embodiments, R^{2.1} is unsubstituted isobutyl. In embodiments, R^{2.1} is unsubstituted tert-butyl. In embodiments, R^{2.1} is unsubstituted 2 to 6 membered heteroalkyl. In embodiments, R^{2.1} is unsubstituted methoxy. In embodiments, R^{2.1} is unsubstituted ethoxy. In embodiments, R^{2.1} is unsubstituted propoxy. In embodiments, R^{2.1} is unsubstituted n-propoxy. In embodiments, R^{2.1} is unsubstituted isopropoxy. In embodiments, R^{2.1} is unsubstituted butoxy.

In embodiments, a substituted R^{2.2} (e.g., substituted alkyl and/or substituted heteroalkyl) is substituted with at least one substituent group, size-limited substituent group, or lower substituent group; wherein if the substituted R^{2.2} is substituted with a plurality of groups selected from substituent groups, size-limited substituent groups, and lower substituent groups; each substituent group, size-limited substituent group, and/or lower substituent group may optionally be different. In embodiments, when R^{2.2} is substituted, it is substituted with at least one substituent group. In embodiments, when R^{2.2} is substituted, it is substituted with at least one size-limited substituent group. In embodiments, when R^{2.2} is substituted, it is substituted with at least one lower substituent group.

In embodiments, R^{2.2} is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, or substituted or unsubstituted heteroalkyl.

In embodiments, R^{2.2} is hydrogen or halogen. In embodiments, R^{2.2} is hydrogen, —Cl, or —F.

In embodiments, R^{2.2} is hydrogen. In embodiments, R^{2.2} is halogen. In embodiments, R^{2.2} is —F. In embodiments, R^{2.2} is —Cl. In embodiments, R^{2.2} is —Br. In embodiments, R^{2.2} is —I. In embodiments, R^{2.2} is —CCl₃. In embodiments, R^{2.2} is —CBr₃. In embodiments, R^{2.2} is —CF₃. In embodiments, R^{2.2} is —Cl₃. In embodiments, R^{2.2} is —CH₂Cl. In embodiments, R^{2.2} is —CH₂Br. In embodiments, R^{2.2} is —CH₂F. In embodiments, R^{2.2} is —CH₂I. In embodiments, R^{2.2} is —CHCl₂. In embodiments, R^{2.2} is —CHBr₂. In embodiments, R^{2.2} is —CHF₂. In embodiments, R^{2.2} is —CHI₂. In embodiments, R^{2.2} is —CN. In embodiments, R^{2.2} is —OH. In embodiments, R^{2.2} is —NH₂. In embodiments, R^{2.2} is —COOH. In embodiments, R^{2.2} is —CONH₂. In embodiments, R^{2.2} is —NO₂. In embodiments, R^{2.2} is —SH. In embodiments, R^{2.2} is —SO₃H. In embodiments, R^{2.2} is —OSO₃H. In embodiments, R^{2.2} is —SO₂NH₂. In embodiments, R^{2.2} is —NHNH₂. In embodiments, R^{2.2} is —ONH₂. In embodiments, R^{2.2} is —NHC(O)NH₂. In embodiments, R^{2.2} is —NHSO₂H. In embodiments, R^{2.2} is —NHC(O)H. In embodiments, R^{2.2} is —NHC(O)OH. In embodiments, R^{2.2} is —NHOH. In embodiments, R^{2.2} is —OCCl₃. In embodiments, R^{2.2} is —OCBr₃. In embodiments, R^{2.2} is —OCF₃. In embodiments, R^{2.2} is —OCI₃. In embodiments, R^{2.2} is —OCH₂Cl. In embodiments, R^{2.2} is —OCH₂Br. In embodiments, R^{2.2} is —OCH₂F. In embodiments, R^{2.2} is —OCH₂I. In embodiments, R^{2.2} is —OCHCl₂. In embodiments, R^{2.2} is —OCHBr₂. In embodiments, R^{2.2} is —OCHF₂. In embodiments, R^{2.2} is —OCHI₂. In embodiments, R^{2.2} is —SF₅. In embodiments, R^{2.2} is —N₃. In embodiments, R^{2.2} is unsubstituted C₁-C₄ alkyl. In embodiments, R^{2.2} is unsubstituted methyl. In embodiments, R^{2.2} is unsubstituted ethyl. In embodiments, R^{2.2} is unsubstituted propyl. In embodiments, R^{2.2} is unsubstituted n-propyl. In embodiments, R^{2.2} is unsubstituted isopropyl. In embodiments, R^{2.2} is unsubstituted butyl. In embodiments, R^{2.2} is unsubstituted n-butyl. In embodiments, R^{2.2} is unsubstituted isobutyl. In embodiments, R^{2.2} is unsubstituted tert-butyl. In embodiments, R^{2.2} is unsubstituted 2 to 6 membered heteroalkyl. In embodiments, R^{2.2} is unsubstituted methoxy. In embodiments, R^{2.2} is unsubstituted ethoxy. In embodiments, R^{2.2} is unsubstituted propoxy. In embodiments, R^{2.2} is unsubstituted n-propoxy. In embodiments, R^{2.2} is unsubstituted isopropoxy. In embodiments, R^{2.2} is unsubstituted butoxy.

In embodiments, R^{2.1} is hydrogen; and R^{2.2} is halogen. In embodiments, R^{2.1} is hydrogen; and R^{2.2} is —Cl. In embodiments, R^{2.1} is halogen; and R^{2.2} is hydrogen. In embodiments, R^{2.1} is —Cl; and R^{2.2} is hydrogen. In embodiments, R^{2.1} and R^{2.2} are halogen. In embodiments, R^{2.1} is —F; and R^{2.2} is —Cl.

In embodiments, a substituted R^{2A} (e.g., substituted alkyl and/or substituted heteroalkyl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{2B} (e.g., substituted alkyl and/or substituted heteroalkyl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, a substituted R^{2C} (e.g., substituted alkyl and/or substituted heteroalkyl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, a substituted R^{2D} (e.g., substituted alkyl and/or substituted heteroalkyl) is substituted with at least one substituent group, size-limited substituent group, or lower substituent group; wherein if the substituted R^{2D} is substituted with a plurality of groups selected from substituent groups, size-limited substituent groups, and lower substituent groups; each substituent group, size-limited substituent group, and/or lower substituent group may optionally be different. In embodiments, when R^{2D} is substituted, it is substituted with at least one substituent group. In embodiments, when R^{2D} is substituted, it is substituted with at least one size-limited substituent group. In embodiments, when R^{2D} is substituted, it is substituted with at least one lower substituent group.

In embodiments, R^{2A} is independently hydrogen. In embodiments, R^{2A} is independently unsubstituted C₁-C₄ alkyl. In embodiments, R^{2A} is independently unsubstituted methyl. In embodiments, R^{2A} is independently unsubstituted ethyl. In embodiments, R^{2A} is independently unsubstituted propyl. In embodiments, R^{2A} is independently unsubstituted n-propyl. In embodiments, R^{2A} is independently unsubstituted isopropyl. In embodiments, R^{2A} is independently unsubstituted butyl. In embodiments, R^{2A} is independently unsubstituted n-butyl. In embodiments, R^{2A} is independently unsubstituted isobutyl. In embodiments, R^{2A} is independently unsubstituted tert-butyl.

In embodiments, R^{2B} is independently hydrogen. In embodiments, R^{2B} is independently unsubstituted C₁-C₄ alkyl. In embodiments, R^{2B} is independently unsubstituted methyl. In embodiments, R^{2B} is independently unsubstituted ethyl. In embodiments, R^{2B} is independently unsubstituted propyl. In embodiments, R^{2B} is independently unsubstituted n-propyl. In embodiments, R^{2B} is independently unsubstituted isopropyl. In embodiments, R^{2B} is independently unsubstituted butyl. In embodiments, R^{2B} is independently unsubstituted n-butyl. In embodiments, R^{2B} is independently unsubstituted isobutyl. In embodiments, R^{2B} is independently unsubstituted tert-butyl.

In embodiments, R^{2C} is independently hydrogen. In embodiments, R^{2C} is independently unsubstituted C₁-C₄ alkyl. In embodiments, R^{2C} is independently unsubstituted methyl. In embodiments, R^{2C} is independently unsubstituted ethyl. In embodiments, R^{2C} is independently unsubstituted propyl. In embodiments, R^{2C} is independently unsubstituted n-propyl. In embodiments, R^{2C} is independently unsubstituted isopropyl. In embodiments, R^{2C} is independently unsubstituted butyl. In embodiments, R^{2C} is independently unsubstituted n-butyl. In embodiments, R^{2C} is independently unsubstituted isobutyl. In embodiments, R^{2C} is independently unsubstituted tert-butyl.

In embodiments, R^{2D} is independently hydrogen. In embodiments, R^{2D} is independently unsubstituted C₁-C₄ alkyl. In embodiments, R^{2D} is independently unsubstituted methyl. In embodiments, R^{2D} is independently unsubstituted ethyl. In embodiments, R^{2D} is independently unsubstituted propyl. In embodiments, R^{2D} is independently unsubstituted n-propyl. In embodiments, R^{2D} is independently unsubstituted isopropyl. In embodiments, R^{2D} is independently unsubstituted butyl. In embodiments, R^{2D} is independently unsubstituted n-butyl. In embodiments, R^{2D} is independently unsubstituted isobutyl. In embodiments, R^{2D} is independently unsubstituted tert-butyl.

In embodiments, when R^{1.1} is substituted, R^{1.1} is substituted with one or more first substituent groups denoted by R^{1.1.1} as explained in the definitions section above in the description of “first substituent group(s)”. In embodiments, when an R^{1.1.1} substituent group is substituted, the R^{1.1.1} substituent group is substituted with one or more second substituent groups denoted by R^{1.1.2} as explained in the definitions section above in the description of “first substituent group(s)”. In embodiments, when an R^{1.1.2} substituent group is substituted, the R^{1.1.2} substituent group is substituted with one or more third substituent groups denoted by R^{1.1.3} as explained in the definitions section above in the description of “first substituent group(s)”. In the above embodiments, R^{1.1}, R^{1.1.1}, R^{1.1.2}, and R^{1.1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{1.1}, R^{1.1.1}, R^{1.1.2}, and R^{1.1.3}, respectively.

In embodiments, when R^{1.2} is substituted, R^{1.2} is substituted with one or more first substituent groups denoted by R^{1.2.1} as explained in the definitions section above in the description of “first substituent group(s)”. In embodiments, when an R^{1.2.1} substituent group is substituted, the R^{1.2.1} substituent group is substituted with one or more second substituent groups denoted by R^{1.2.2} as explained in the definitions section above in the description of “first substituent group(s)”. In embodiments, when an R^{1.2.2} substituent group is substituted, the R^{1.2.2} substituent group is substituted with one or more third substituent groups denoted by R^{1.2.3} as explained in the definitions section above in the description of “first substituent group(s)”. In the above embodiments, R^{1.2}, R^{1.2.1}, R^{1.2.2}, and R^{1.2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{1.2}, R^{1.2.1}, R^{1.2.2}, and R^{1.2.3}, respectively.

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to RIA, R^{1A.1}, R^{1A.2}, and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2}, and R^{1B.3} have values corresponding to the values of R^{WW}, R^{WW.1}. R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{1B}, R^{1B.1}, R^{1B.2}, and R^{1B.3}, respectively.

In embodiments, when R^{1C} is substituted, R^{1C} is substituted with one or more first substituent groups denoted by R^{1C.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, R^{1C}, R^{1C.1}, R^{1C.2}, and R^{1C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{1C}, R^{1C.1}, R^{1C.2}, and R^{1C.3}, respectively.

In embodiments, when R^{1D} is substituted, R^{1D} is substituted with one or more first substituent groups denoted by R^{1D.1} as explained in the definitions section above in the description of “first substituent group(s)”. In embodiments, when an R^{1D.1} substituent group is substituted, the R^{1D.1} substituent group is substituted with one or more second substituent groups denoted by R^{1D.2} as explained in the definitions section above in the description of “first substituent group(s)”. In embodiments, when an R^{1D.2} substituent group is substituted, the R^{1D.2} substituent group is substituted with one or more third substituent groups denoted by R^{1D.3} as explained in the definitions section above in the description of “first substituent group(s)”. In the above embodiments, R^{1D}, R^{1D.1}, R^{1D.2}, and R^{1D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{1D}, R^{1D.1}, R^{1D.2}, and R^{1D.3}, respectively.

In embodiments, when R^{2.1} is substituted, R^{2.1} is substituted with one or more first substituent groups denoted by R^{2.1.1} as explained in the definitions section above in the description of “first substituent group(s)”. In embodiments, when an R^{2.1.1} substituent group is substituted, the R^{2.1.1} substituent group is substituted with one or more second substituent groups denoted by R^{2.1.2} as explained in the definitions section above in the description of “first substituent group(s)”. In embodiments, when an R^{2.1.2} substituent group is substituted, the R^{2.1.2} substituent group is substituted with one or more third substituent groups denoted by R^{2.1.3} as explained in the definitions section above in the description of “first substituent group(s)”. In the above embodiments, R^{2.1}, R^{2.1.1}, R^{2.1.2}, and R^{2.1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{2.1}, R^{2.1.1}, R^{2.1.2}, and R^{2.1.3}, respectively.

In embodiments, when R^{2.2} is substituted, R^{2.2} is substituted with one or more first substituent groups denoted by R^{2.2.1} as explained in the definitions section above in the description of “first substituent group(s)”. In embodiments, when an R^{2.2.1} substituent group is substituted, the R^{2.2.1} substituent group is substituted with one or more second substituent groups denoted by R^{2.2.2} as explained in the definitions section above in the description of “first substituent group(s)”. In embodiments, when an R^{2.2.2} substituent group is substituted, the R^{2.2.2} substituent group is substituted with one or more third substituent groups denoted by R^{2.2.3} as explained in the definitions section above in the description of “first substituent group(s)”. In the above embodiments, R^{2.2}, R^{2.2.1}, R^{2.2.2}, and R^{2.2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{2.2}, R^{2.2.1}, R^{2.2.2}, and R^{2.2.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{2B}, R^{2B.1}. R^{2B.2}, and R^{2B.3} respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{2C}, R^{2C.1}, R^{2C.2}, and R^{2C.3}, respectively.

In embodiments, when R^{2D} is substituted, R^{2D} is substituted with one or more first substituent groups denoted by R^{2D.1} as explained in the definitions section above in the description of “first substituent group(s)”. In embodiments, when an R^{2D.1} substituent group is substituted, the R^{2D.1} substituent group is substituted with one or more second substituent groups denoted by R^{2D.2} as explained in the definitions section above in the description of “first substituent group(s)”. In embodiments, when an R^{2D.2} substituent group is substituted, the R^{2D.2} substituent group is substituted with one or more third substituent groups denoted by R^{2D.3} as explained in the definitions section above in the description of “first substituent group(s)”. In the above embodiments, R^{2D}, R^{2D.1}, R^{2D.2}, and R^{2D.3} have values corresponding to the values of R^{WW}, R^{WW.1}. R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{2D}, R^{2D.1}, R^{2D.2}, and R^{2D.3}, respectively.

In embodiments, the compound has the formula:

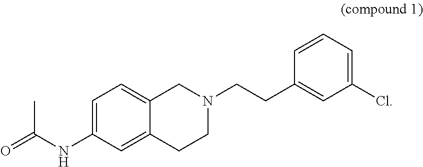 [SMILES: CC(=O)Nc1ccc2c(c1)CCN(CCc1cccc(Cl)c1)C2]

In embodiments, the compound has the formula:

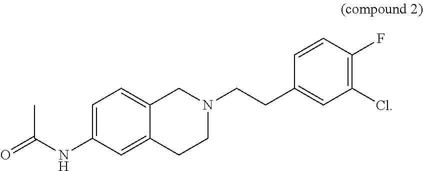 [SMILES: CC(=O)Nc1ccc2c(c1)CCN(CCc1ccc(F)c(Cl)c1)C2]

In embodiments, the compound has the formula:

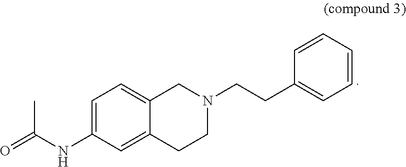 [SMILES: CC(=O)Nc1ccc2c(c1)CCN(CCc1ccccc1)C2]

In embodiments, the compound has the formula:

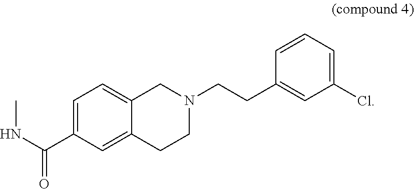 [SMILES: CNC(=O)c1ccc2c(c1)CCN(CCc1cccc(Cl)c1)C2]

In embodiments, the compound has the formula:

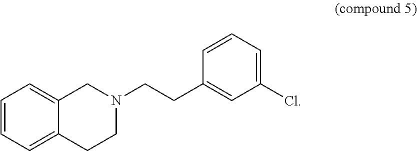 [SMILES: Clc1cccc(CCN2CCc3ccccc3C2)c1]

In embodiments, the compound has the formula:

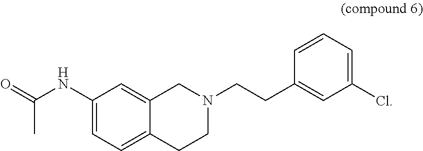 [SMILES: CC(=O)Nc1ccc2c(c1)CN(CCc1cccc(Cl)c1)CC2]

In embodiments, the compound has the formula:

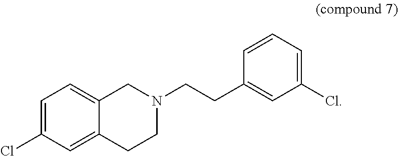 [SMILES: Clc1cccc(CCN2CCc3cc(Cl)ccc3C2)c1]

In embodiments, the compound has the formula:

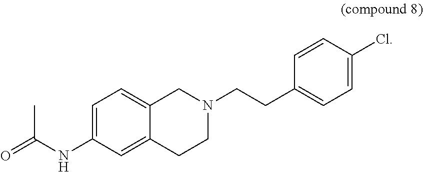 [SMILES: CC(=O)Nc1ccc2c(c1)CCN(CCc1ccc(Cl)cc1)C2]

In embodiments, the compound has the formula:

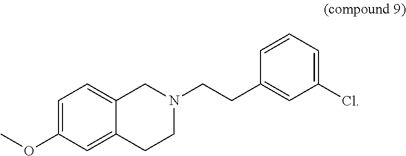 [SMILES: COc1ccc2c(c1)CCN(CCc1cccc(Cl)c1)C2]

In embodiments, the compound has the formula:

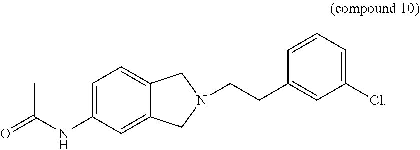 [SMILES: CC(=O)Nc1ccc2c(c1)CN(CCc1cccc(Cl)c1)C2]

In embodiments, the compound has the formula:

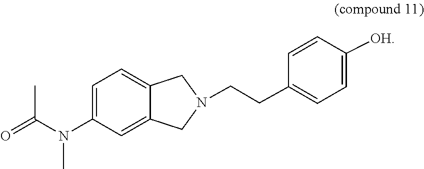 [SMILES: CC(=O)N(C)c1ccc2c(c1)CN(CCc1ccc(O)cc1)C2]

In embodiments, the compound has the formula:

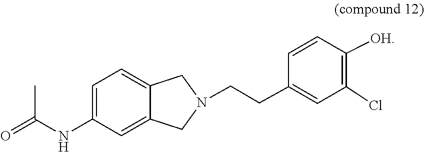 [SMILES: CC(=O)Nc1ccc2c(c1)CN(CCc1ccc(O)c(Cl)c1)C2]

In embodiments, the compound has the formula:

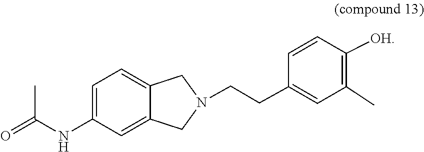 [SMILES: CC(=O)Nc1ccc2c(c1)CN(CCc1ccc(O)c(C)c1)C2]

In embodiments, the compound has the formula:

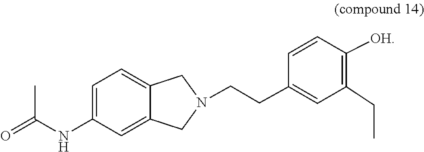 [SMILES: CCc1cc(CCN2Cc3ccc(NC(C)=O)cc3C2)ccc1O]

In embodiments, the compound has the formula:

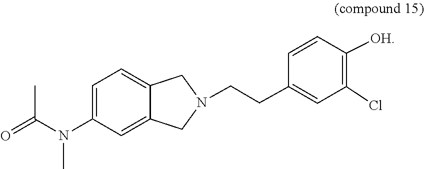 [SMILES: CC(=O)N(C)c1ccc2c(c1)CN(CCc1ccc(O)c(Cl)c1)C2]

In embodiments, the compound has the formula:

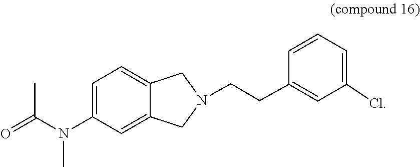 [SMILES: CC(=O)N(C)c1ccc2c(c1)CN(CCc1cccc(Cl)c1)C2]

In embodiments, R^{1.1} and R^{1.2} are not halogen, —C(O)NR^{1A}R^{1B}, or —NR^{1A}C(O)R^{1C}. In embodiments, R^{1.1} and R^{1.2} are not-NHC(O) CH₃. In embodiments, R^{1.1} and R^{1.2} are not —C(O)NHCH₃. In embodiments, R^{1.1} and R^{1.2} are not —OR^{1D}. In embodiments, R^{1.1} and R^{1.2} are not —OCH₃.

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

### III. Pharmaceutical Compositions

In an aspect is provided a pharmaceutical composition including a compound described herein, or a pharmaceutically acceptable salt thereof, and a pharmaceutically acceptable excipient.

In embodiments, the pharmaceutical composition includes an effective amount of the compound. In embodiments, the pharmaceutical composition includes a therapeutically effective amount of the compound.

In embodiments, the compound is a compound of formula (I) or (Ia), including all embodiments thereof. In embodiments, the compound is a compound as described herein, including in embodiments. In embodiments the compound is a compound described herein (e.g., in the examples section, figures, tables, or claims).

In embodiments, the pharmaceutical composition is an oral dosage form. In embodiments, the pharmaceutical composition is a tablet dosage form. In embodiments, the pharmaceutical composition is a capsule dosage form. In embodiments, the pharmaceutical composition is an intranasal dosage form. In embodiments, the pharmaceutical composition is an intravenous dosage form. In embodiments, the pharmaceutical composition is an injectable dosage form.

### IV. Methods of Use

In an aspect is provided a method of treating a protein aggregation disease in a subject in need thereof, the method including administering to the subject a therapeutically effective amount of a compound described herein, or a pharmaceutically acceptable salt thereof.

In embodiments, the compound is a compound of formula (I) or (Ia), including all embodiments thereof. In embodiments, the compound is a compound as described herein, including in embodiments. In embodiments the compound is a compound described herein (e.g., in the examples section, figures, tables, or claims).

In embodiments, the protein aggregation disease is a neurodegenerative disease. In embodiments, the neurodegenerative disease is frontotemporal dementia. In embodiments, the neurodegenerative disease is a synucleinopathy. In embodiments, the neurodegenerative disease is a tauopathy. In embodiments, the neurodegenerative disease is amyotrophic lateral sclerosis (ALS). In embodiments, the neurodegenerative disease is sporadic amyotrophic lateral sclerosis. In embodiments, the neurodegenerative disease is TDP-43 mutated amyotrophic lateral sclerosis. In embodiments, the neurodegenerative disease is amyotrophic lateral sclerosis and frontotemporal lobar degeneration. In embodiments, the neurodegenerative disease is frontotemporal lobar degeneration. In embodiments, the neurodegenerative disease is Pick's disease. In embodiments, the neurodegenerative disease is frontotemporal lobar degeneration TDP-43 predominant pathology. In embodiments, the neurodegenerative disease is frontotemporal lobar degeneration tau predominant pathology. In embodiments, the neurodegenerative disease is semantic dementia. In embodiments, the neurodegenerative disease is primary progressive aphasia. In embodiments, the neurodegenerative disease is multiple system proteinopathy. In embodiments, the neurodegenerative disease is Perry syndrome. In embodiments, the neurodegenerative disease is facial onset sensory and motor neuropathy. In embodiments, the neurodegenerative disease is valosin containing protein (VCP) disease. In embodiments, the neurodegenerative disease is inclusion body myositis. In embodiments, the neurodegenerative disease is myotonic disorder. In embodiments, the neurodegenerative disease is Alzheimer's disease. In embodiments, the neurodegenerative disease is Parkinson's disease. In embodiments, the neurodegenerative disease is dementia with Lewy bodies. In embodiments, the neurodegenerative disease is Parkinson's disease-associated dementia. In embodiments, the neurodegenerative disease is multiple system atrophy. In embodiments, the neurodegenerative disease is corticobasal degeneration. In embodiments, the neurodegenerative disease is progressive supranuclear palsy. In embodiments, the neurodegenerative disease is Huntington's disease. In embodiments, the neurodegenerative disease is C9orf72 amyotrophic lateral sclerosis. In embodiments, the neurodegenerative disease is amyloidosis. In embodiments, the neurodegenerative disease is antibody light chain amyloidosis. In embodiments, the neurodegenerative disease is transthyretin amyloidosis. In embodiments, the neurodegenerative disease is prion disease. In embodiments, the prion disease is Creutzfeldt-Jakob disease. In embodiments, the prion disease is variant Creutzfeldt-Jakob disease. In embodiments, the prion disease is genetic human prion disease. In embodiments, the prion disease is bovine spongiform encephalopathy. In embodiments, the prion disease is scrapie. In embodiments, the neurodegenerative disease is diffuse Lewy body disease. In embodiments, the neurodegenerative disease is spinocerebellar ataxia. In embodiments, the neurodegenerative disease is poly-Q disease. In embodiments, the neurodegenerative disease is hereditary cerebral amyloid angiopathy. In embodiments, the neurodegenerative disease is familial amyloid polyneuropathy. In embodiments, the neurodegenerative disease is C9orf72 frontotemporal dementia. In embodiments, the neurodegenerative disease is FUS ALS and FUS frontotemporal dementia. In embodiments, the neurodegenerative disease is TMEM106b Parkinson's disease. In embodiments, the neurodegenerative disease is TMEM106b Alzheimer's disease. In embodiments, the neurodegenerative disease is TMEM106b ALS. In embodiments, the neurodegenerative disease is TMEM175 Parkinson's disease. In embodiments, the neurodegenerative disease is TMEM175 Alzheimer's disease. In embodiments, the neurodegenerative disease is TMEM175 Alzheimer's disease.

In embodiments, the protein aggregation disease is diabetes. In embodiments, the protein aggregation disease is type II diabetes. In embodiments, the protein aggregation disease is type I diabetes. In embodiments, the protein aggregation disease is a p53 mutant cancer. In embodiments, the protein aggregation disease is an influenza A virus infection. In embodiments, the protein aggregation disease is a herpesvirus infection. In embodiments, the herpesvirus is an M45 herpesvirus infection. In embodiments, the protein aggregation disease is primary systemic amyloidosis. In embodiments, the protein aggregation disease is reactive systemic amyloidosis. In embodiments, the protein aggregation disease is injection-localized amyloidosis. In embodiments, the protein aggregation disease is beta-2 microglobulin amyloidosis. In embodiments, the protein aggregation disease is hereditary non-neuropathic amyloidosis. In embodiments, the protein aggregation disease is Finnish hereditary systemic amyloidosis.

In embodiments, the compound is administered orally. In embodiments, the compound is administered intranasally. In embodiments, the compound is administered intravenously. In embodiments, the compound is administered by injection.

In an aspect is provided a method of reducing formation of a protein aggregate or disrupting a protein aggregate, the method including contacting the protein aggregate with an effective amount of a compound described herein, or a pharmaceutically acceptable salt thereof.

In embodiments, the compound is a compound of formula (I) or (Ia), including all embodiments thereof. In embodiments, the compound is a compound as described herein, including in embodiments. In embodiments the compound is a compound described herein (e.g., in the examples section, figures, tables, or claims).

In embodiments, the protein aggregate is a prion protein aggregate. In embodiments, the protein aggregate is an amyloid precursor protein aggregate. In embodiments, the protein aggregate is a beta amyloid aggregate. In embodiments, the protein aggregate is an alpha synuclein aggregate. In embodiments, the protein aggregate is a superoxide dismutase aggregate. In embodiments, the protein aggregate is a tau aggregate. In embodiments, the protein aggregate is an immunoglobulin aggregate. In embodiments, the protein aggregate is an amyloid-A aggregate. In embodiments, the protein aggregate is a transthyretin aggregate. In embodiments, the protein aggregate is a beta-2 microglobulin aggregate. In embodiments, the protein aggregate is a cystatin C aggregate. In embodiments, the protein aggregate is an apolipoprotein A1 aggregate. In embodiments, the protein aggregate is a TDP-43 aggregate. In embodiments, the protein aggregate is an islet amyloid polypeptide aggregate. In embodiments, the protein aggregate is an ANF aggregate. In embodiments, the protein aggregate is a gelsolin aggregate. In embodiments, the protein aggregate is an insulin aggregate. In embodiments, the protein aggregate is a lysozyme aggregate. In embodiments, the protein aggregate is a fibrinogen aggregate. In embodiments, the protein aggregate is a huntingtin aggregate. In embodiments, the protein aggregate is an ataxin aggregate. In embodiments, the protein aggregate is an aggregate of a protein with a Poly-Q stretch. In embodiments, the protein with a Poly-Q stretch is a protein having at least 36 consecutive glutamine residues.

In embodiments, the protein aggregate is an oligomer. In embodiments, the oligomer is a trimer. In embodiments, the oligomer is a tetramer. In embodiments, the oligomer is a pentamer. In embodiments, the oligomer is a hexamer. In embodiments, the oligomer is a heptamer. In embodiments, the oligomer is an octamer. In embodiments, the oligomer is a nonamer. In embodiments, the oligomer is a decamer. In embodiments, the oligomer is an 11-mer. In embodiments, the oligomer is a 12-mer. In embodiments, the oligomer is a 13-mer. In embodiments, the oligomer is a 14-mer. In embodiments, the oligomer is a 15-mer. In embodiments, the oligomer is a 16-mer. In embodiments, the oligomer is a 17-mer. In embodiments, the oligomer is an 18-mer. In embodiments, the oligomer is a 19-mer. In embodiments, the oligomer is a 20-mer.

In embodiments, the protein aggregate is an oligomer. In embodiments, the oligomer has a size range of from about 40 kD to about 500 kD. In embodiments, the oligomer has a size range of from about 50 kD to about 400 kD. In embodiments, the oligomer has a size range of from about 65 kD to about 150 kD. In embodiments, the oligomer has a size range of from about 100 kD to about 300 kD. In embodiments, the oligomer has a size range of from about 45 kD to about 200 kD. In embodiments, the oligomer has a size range of from about 80 kD to about 500 kD. In embodiments, the oligomer is about 40 kD. In embodiments, the oligomer is about 50 kD. In embodiments, the oligomer is about 60 kD. In embodiments, the oligomer is about 70 kD. In embodiments, the oligomer is about 80 kD. In embodiments, the oligomer is about 90 kD. In embodiments, the oligomer is about 100 kD. In embodiments, the oligomer is about 120 kD. In embodiments, the oligomer is about 140 kD. In embodiments, the oligomer is about 160 kD. In embodiments, the oligomer is about 180 kD. In embodiments, the oligomer is about 200 kD. In embodiments, the oligomer is about 220 kD. In embodiments, the oligomer is about 240 kD. In embodiments, the oligomer is about 260 kD. In embodiments, the oligomer is about 280 kD. In embodiments, the oligomer is about 300 kD. In embodiments, the oligomer is about 320 kD. In embodiments, the oligomer is about 340 kD. In embodiments, the oligomer is about 360 kD. In embodiments, the oligomer is about 380 kD. In embodiments, the oligomer is about 400 kD. In embodiments, the oligomer is about 420 kD. In embodiments, the oligomer is about 440 kD. In embodiments, the oligomer is about 460 kD. In embodiments, the oligomer is about 480 kD. In embodiments, the oligomer is about 500 kD.

In embodiments, the protein aggregate is an oligomer. In embodiments, the oligomer has a size range of from 40 kD to 500 kD. In embodiments, the oligomer has a size range of from 50 kD to 400 kD. In embodiments, the oligomer has a size range of from 65 kD to 150 kD. In embodiments, the oligomer has a size range of from 100 kD to 300 kD. In embodiments, the oligomer has a size range of from 45 kD to 200 kD. In embodiments, the oligomer has a size range of from 80 kD to 500 kD. In embodiments, the oligomer is 40 kD. In embodiments, the oligomer is 50 kD. In embodiments, the oligomer is 60 kD. In embodiments, the oligomer is 70 kD. In embodiments, the oligomer is 80 kD. In embodiments, the oligomer is 90 kD. In embodiments, the oligomer is 100 kD. In embodiments, the oligomer is 120 kD. In embodiments, the oligomer is 140 kD. In embodiments, the oligomer is 160 kD. In embodiments, the oligomer is 180 kD. In embodiments, the oligomer is 200 kD. In embodiments, the oligomer is 220 kD. In embodiments, the oligomer is 240 kD. In embodiments, the oligomer is 260 kD. In embodiments, the oligomer is 280 kD. In embodiments, the oligomer is 300 kD. In embodiments, the oligomer is 320 kD. In embodiments, the oligomer is 340 kD. In embodiments, the oligomer is 360 kD. In embodiments, the oligomer is 380 kD. In embodiments, the oligomer is 400 kD. In embodiments, the oligomer is 420 kD. In embodiments, the oligomer is 440 kD. In embodiments, the oligomer is 460 kD. In embodiments, the oligomer is 480 kD. In embodiments, the oligomer is 500 kD.

In embodiments, the contacting occurs in vivo. In embodiments, the contacting occurs in a subject, wherein an effective amount of the compound is administered to the subject. In embodiments, the subject has a protein aggregation disease. In embodiments, the contacting occurs in vitro. In embodiments, the contacting occurs ex vivo.

In embodiments, the amount of the protein aggregate is decreased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the amount of the protein aggregate is decreased by about 1.5-fold relative to a control (e.g., absence of the compound). In embodiments, the amount of the protein aggregate is decreased by about 2-fold relative to a control (e.g., absence of the compound). In embodiments, the amount of the protein aggregate is decreased by about 5-fold relative to a control (e.g., absence of the compound). In embodiments, the amount of the protein aggregate is decreased by about 10-fold relative to a control (e.g., absence of the compound). In embodiments, the amount of the protein aggregate is decreased by about 25-fold relative to a control (e.g., absence of the compound). In embodiments, the amount of the protein aggregate is decreased by about 50-fold relative to a control (e.g., absence of the compound). In embodiments, the amount of the protein aggregate is decreased by about 100-fold relative to a control (e.g., absence of the compound). In embodiments, the amount of the protein aggregate is decreased by about 250-fold relative to a control (e.g., absence of the compound). In embodiments, the amount of the protein aggregate is decreased by about 500-fold relative to a control (e.g., absence of the compound). In embodiments, the amount of the protein aggregate is decreased by about 1000-fold relative to a control (e.g., absence of the compound).

In embodiments, the amount of the protein aggregate is decreased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the amount of the protein aggregate is decreased by at least 1.5-fold relative to a control (e.g., absence of the compound). In embodiments, the amount of the protein aggregate is decreased by at least 2-fold relative to a control (e.g., absence of the compound). In embodiments, the amount of the protein aggregate is decreased by at least 5-fold relative to a control (e.g., absence of the compound). In embodiments, the amount of the protein aggregate is decreased by at least 10-fold relative to a control (e.g., absence of the compound). In embodiments, the amount of the protein aggregate is decreased by at least 25-fold relative to a control (e.g., absence of the compound). In embodiments, the amount of the protein aggregate is decreased by at least 50-fold relative to a control (e.g., absence of the compound). In embodiments, the amount of the protein aggregate is decreased by at least 100-fold relative to a control (e.g., absence of the compound). In embodiments, the amount of the protein aggregate is decreased by at least 250-fold relative to a control (e.g., absence of the compound). In embodiments, the amount of the protein aggregate is decreased by at least 500-fold relative to a control (e.g., absence of the compound). In embodiments, the amount of the protein aggregate is decreased by at least 1000-fold relative to a control (e.g., absence of the compound).

### V. Embodiments

Embodiment P1. A compound, or a pharmaceutically acceptable salt thereof, having the formula:

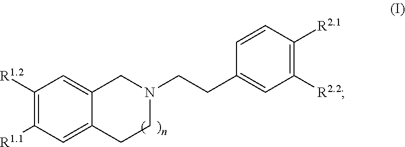

wherein
n is 0 or 1;
R^{1.1} and R^{1.2} are independently hydrogen, halogen, —C(O)NR^{1A}R^{1B}, or —NR^{1A}C(O)R^{1C};
R^{2.1} and R^{2.2} are independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NR^{2C}C(O)NR^{2A}R^{2B}, —N(O)ₘ₂, —NR^{2A}R^{2B}, —C(O)R^{2C}, —C(O) OR^{2C}, —OC(O)R^{2C}, —OC(O)OR^{2C}, —C(O)NR^{2A}R^{2B}, —OC(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, or substituted or unsubstituted heteroalkyl;

R^{1A}, R^{1B}, R^{1C}, R^{2A}, R^{2B}, R^{2C}, and R^{2D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
each X² is independently —F, —Cl, —Br, or —I;
n2 is an integer from 0 to 4; and
m2 and v2 are independently 1 or 2;
wherein at least one of R^{1.1} and R^{1.2} is not hydrogen; and
wherein the compound is not:

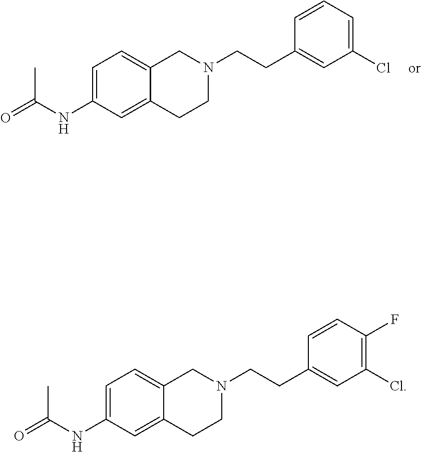 [SMILES: CC(=O)Nc1ccc2c(c1)CCN(CCc1cccc(Cl)c1)C2 ; CC(=O)Nc1ccc2c(c1)CCN(CCc1ccc(F)c(Cl)c1)C2]

Embodiment P2. The compound of embodiment P1, wherein R^{1.1} is halogen, —C(O)NR^{1A}R^{1B}, or —NR^{1A}C(O)R^{1C}; and R^{1.2} is hydrogen.

Embodiment P3. The compound of embodiment P2, wherein R^{1A} and R^{1B} are independently hydrogen or unsubstituted C₁-C₄ alkyl, and R^{1C} is unsubstituted C₁-C₄ alkyl.

Embodiment P4. The compound of embodiment P1, wherein R^{1.1} is —NHC(O) CH₃; and R^{1.2} is hydrogen.

Embodiment P5. The compound of embodiment P1, wherein R^{1.1} is —C(O)NHCH₃; and R^{1.2} is hydrogen.

Embodiment P6. The compound of embodiment P1, wherein R^{1.1} is hydrogen; and R^{1.2} is halogen, —C(O)NR^{1A}R^{1B}, or —NR^{1A}C(O)R^{1C}.

Embodiment P7. The compound of embodiment P6, wherein R^{1A} and R^{1B} are independently hydrogen or unsubstituted C₁-C₄ alkyl, and R^{1C} is unsubstituted C₁-C₄ alkyl.

Embodiment P8. The compound of embodiment P1, wherein R^{1.1} is hydrogen; and R^{1.2} is —NHC(O) CH₃.

Embodiment P9. The compound of one of embodiments P1 to P8, wherein R^{2.1} and R^{2.2} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, or substituted or unsubstituted heteroalkyl.

Embodiment P10. The compound of one of embodiments P1 to P8, wherein R^{2.1} and R^{2.2} are independently hydrogen or halogen.

Embodiment P11. The compound of one of embodiments P1 to P8, wherein R^{2.1} and R^{2.2} are independently hydrogen, —Cl, or —F.

Embodiment P12. The compound of one of embodiments P1 to P11, wherein n is 0.

Embodiment P13. The compound of one of embodiments P1 to P11, wherein n is 1.

Embodiment P14. The compound of embodiment P1, having the formula:

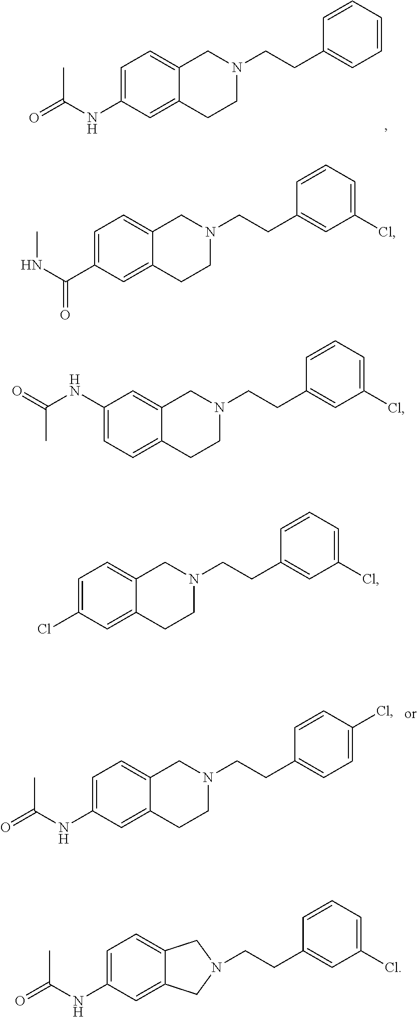 [SMILES: CC(=O)Nc1ccc2c(c1)CCN(CCc1ccccc1)C2 ; CNC(=O)c1ccc2c(c1)CCN(CCc1cccc(Cl)c1)C2 ; CC(=O)Nc1ccc2c(c1)CN(CCc1cccc(Cl)c1)CC2 ; Clc1cccc(CCN2CCc3cc(Cl)ccc3C2)c1 ; CC(=O)Nc1ccc2c(c1)CCN(CCc1ccc(Cl)cc1)C2 ; CC(=O)Nc1ccc2c(c1)CN(CCc1cccc(Cl)c1)C2]

Embodiment P15. A compound, or a pharmaceutically acceptable salt thereof, having the formula:

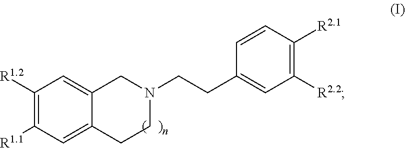

wherein
n is 0 or 1;
R^{1.1} and R^{1.2} are independently hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O) OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, or substituted or unsubstituted heteroalkyl;
R^{2.1} and R^{2.2} are independently hydrogen or halogen;
R^{1A}, R^{1B}, R^{1C}, and R^{1D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
each X¹ is independently —F, —Cl, —Br, or —I;
n1 is an integer from 0 to 4; and
m1 and v1 are independently 1 or 2;
wherein at least one of R^{2.1} and R^{2.2} is not hydrogen; and
wherein the compound is not:

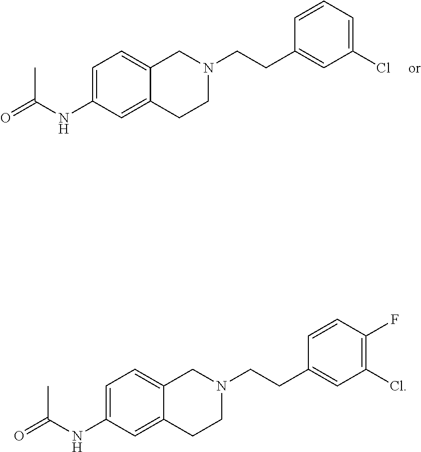 [SMILES: CC(=O)Nc1ccc2c(c1)CCN(CCc1cccc(Cl)c1)C2 ; CC(=O)Nc1ccc2c(c1)CCN(CCc1ccc(F)c(Cl)c1)C2]

Embodiment P16. The compound of embodiment P15, wherein R^{2.1} is hydrogen; and R^{2.2} is halogen.

Embodiment P17. The compound of embodiment P15, wherein R^{2.1} is hydrogen; and R^{2.2} is —Cl.

Embodiment P18. The compound of embodiment P15, wherein R^{2.1} is halogen; and R^{2.2} is hydrogen.

Embodiment P19. The compound of embodiment P15, wherein R^{2.1} is —Cl; and R^{2.2} is hydrogen.

Embodiment P20. The compound of embodiment P15, wherein R^{2.1} and R^{2.2} are halogen.

Embodiment P21. The compound of embodiment P15, wherein R^{2.1} is —F; and R^{2.2} is —Cl.

Embodiment P22. The compound of one of embodiments P15 to P21, wherein R^{1.1} and R^{1.2} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, or substituted or unsubstituted heteroalkyl.

Embodiment P23. The compound of one of embodiments P15 to P21, wherein R^{1.1} and R^{1.2} are independently hydrogen or —OR^{1D}.

Embodiment P24. The compound of embodiment P23, wherein R^{1D} is independently hydrogen or unsubstituted C₁-C₄ alkyl.

Embodiment P25. The compound of one of embodiments P15 to P21, wherein R^{1.1} and R^{1.2} are independently hydrogen or —OCH₃.

Embodiment P26. The compound of one of embodiments P15 to P25, wherein n is 0.

Embodiment P27. The compound of one of embodiments P15 to P25, wherein n is 1.

Embodiment P28. The compound of embodiment P15, having the formula:

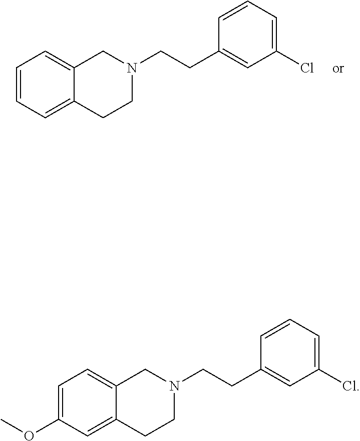 [SMILES: Clc1cccc(CCN2CCc3ccccc3C2)c1 ; COc1ccc2c(c1)CCN(CCc1cccc(Cl)c1)C2]

Embodiment P29. A pharmaceutical composition comprising a pharmaceutically acceptable excipient and the compound of one of embodiments P1 to P28, or a pharmaceutically acceptable salt thereof, or a compound having the formula:

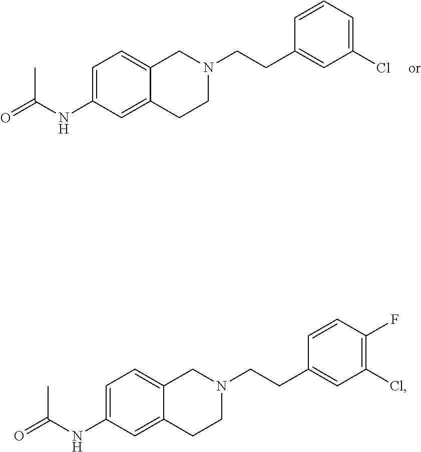 [SMILES: CC(=O)Nc1ccc2c(c1)CCN(CCc1cccc(Cl)c1)C2 ; CC(=O)Nc1ccc2c(c1)CCN(CCc1ccc(F)c(Cl)c1)C2]

or a pharmaceutically acceptable salt thereof.

Embodiment P30. The pharmaceutical composition of embodiment P29, wherein the pharmaceutical composition is an oral dosage form, an intranasal dosage form, an intravenous dosage form, or an injectable dosage form.

Embodiment P31. A method of treating a protein aggregation disease in a subject in need thereof, said method comprising administering to the subject a therapeutically effective amount of the compound of one of embodiments P1 to P28, or a pharmaceutically acceptable salt thereof, or a compound having the formula:

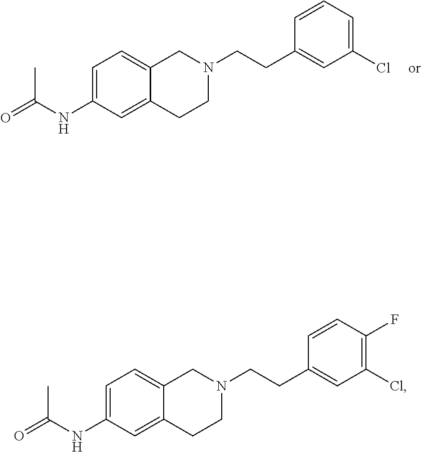 [SMILES: CC(=O)Nc1ccc2c(c1)CCN(CCc1cccc(Cl)c1)C2 ; CC(=O)Nc1ccc2c(c1)CCN(CCc1ccc(F)c(Cl)c1)C2]

or a pharmaceutically acceptable salt thereof.

Embodiment P32. The method of embodiment P31, wherein the protein aggregation disease is a neurodegenerative disease.

Embodiment P33. The method of embodiment P32, wherein the neurodegenerative disease is frontotemporal dementia, a synucleinopathy, or a tauopathy.

Embodiment P34. The method of embodiment P32, wherein the neurodegenerative disease is sporadic amyotrophic lateral sclerosis, amyotrophic lateral sclerosis and frontotemporal lobar degeneration, frontotemporal lobar degeneration, Pick's disease, frontotemporal lobar degeneration TDP-43 predominant pathology, frontotemporal lobar degeneration tau predominant pathology, semantic dementia, primary progressive aphasia, multiple system proteinopathy, Perry syndrome, facial onset sensory and motor neuropathy, sporadic inclusion body myositis, myotonic disorder, Alzheimer's disease, Parkinson's disease, dementia with Lewy bodies, Parkinson's disease-associated dementia, multiple system atrophy, corticobasal degeneration, progressive supranuclear palsy, Huntington's disease, C9orf72 amyotrophic lateral sclerosis, amyloidosis, antibody light chain amyloidosis, transthyretin amyloidosis, prion disease, diffuse Lewy body disease, spinocerebellar ataxia, poly-Q disease, hereditary cerebral amyloid angiopathy, or familial amyloid polyneuropathy.

Embodiment P35. The method of embodiment P34, wherein the prion disease is Creutzfeldt-Jakob disease, variant Creutzfeldt-Jakob disease, genetic human prion disease, bovine spongiform encephalopathy, or scrapie.

Embodiment P36. The method of embodiment P31, wherein the protein aggregation disease is diabetes.

Embodiment P37. The method of embodiment P31, wherein the protein aggregation disease is a p53 mutant cancer.

Embodiment P38. The method of embodiment P31, wherein the protein aggregation disease is an influenza A virus infection or a herpesvirus infection.

Embodiment P39. The method of embodiment P38, wherein the herpesvirus is an M45 herpesvirus infection.

Embodiment P40. The method of embodiment P31, wherein the protein aggregation disease is primary systemic amyloidosis, reactive systemic amyloidosis, type II diabetes, injection-localized amyloidosis, beta-2 microglobulin amyloidosis, hereditary non-neuropathic amyloidosis, or Finnish hereditary systemic amyloidosis.

Embodiment P41. The method of one of embodiments P31 to P40, wherein the compound is administered orally, intranasally, intravenously, or by injection.

Embodiment P42. A method of reducing formation of a protein aggregate or disrupting a protein aggregate, said method comprising contacting the protein aggregate with an effective amount of the compound of one of embodiments P1 to P28, or a pharmaceutically acceptable salt thereof, or a compound having the formula:

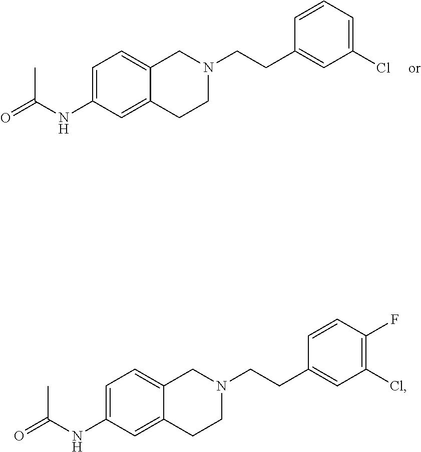 [SMILES: CC(=O)Nc1ccc2c(c1)CCN(CCc1cccc(Cl)c1)C2 ; CC(=O)Nc1ccc2c(c1)CCN(CCc1ccc(F)c(Cl)c1)C2]

or a pharmaceutically acceptable salt thereof.

Embodiment P43. The method of embodiment P42, wherein the protein aggregate is a prion protein aggregate, amyloid precursor protein aggregate, beta amyloid aggregate, alpha synuclein aggregate, superoxide dismutase aggregate, tau aggregate, immunoglobulin aggregate, amyloid-A aggregate, transthyretin aggregate, beta-2 microglobulin aggregate, cystatin C aggregate, apolipoprotein A1 aggregate, TDP-43 aggregate, islet amyloid polypeptide aggregate, ANF aggregate, gelsolin aggregate, insulin aggregate, lysozyme aggregate, fibrinogen aggregate, huntingtin aggregate, or ataxin aggregate.

Embodiment P44. The method of embodiment P42, wherein the protein aggregate is a beta amyloid aggregate.

Embodiment P45. The method of embodiment P42, wherein the protein aggregate is a tau aggregate.

Embodiment P46. The method of embodiment P42, wherein the protein aggregate is an alpha synuclein aggregate.

Embodiment P47. The method of embodiment P42, wherein the protein aggregate is a TDP-43 aggregate.

Embodiment P48. The method of one of embodiments P42 to P47, wherein the protein aggregate is an oligomer.

Embodiment P49. The method of one of embodiments P42 to P48, wherein the contacting occurs in vivo.

Embodiment P50. The method of one of embodiments P42 to P48, wherein the contacting occurs in a subject, wherein an effective amount of the compound is administered to the subject.

Embodiment P51. The method of embodiment P50, wherein the subject has a protein aggregation disease.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Atomic Force Microscopy (AFM) Methods for Amyloid Beta Peptide Protein Aggregation

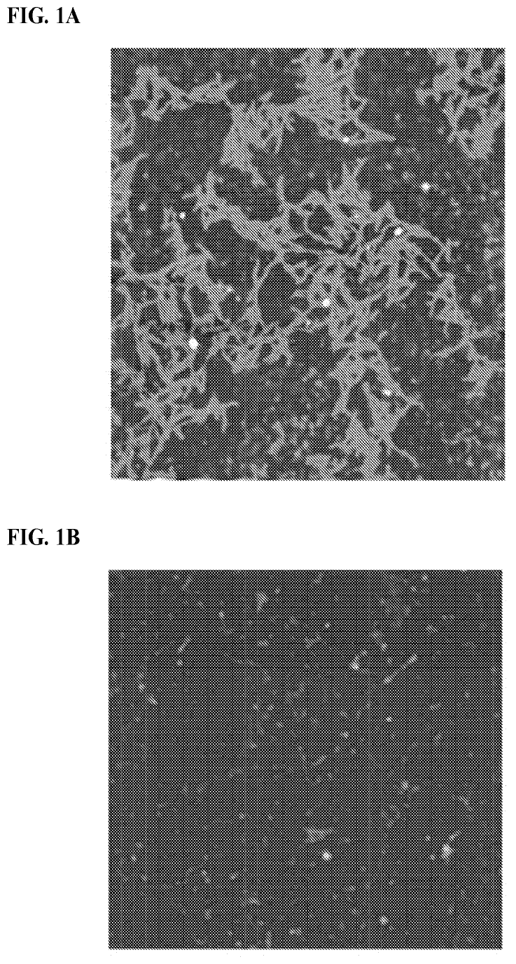

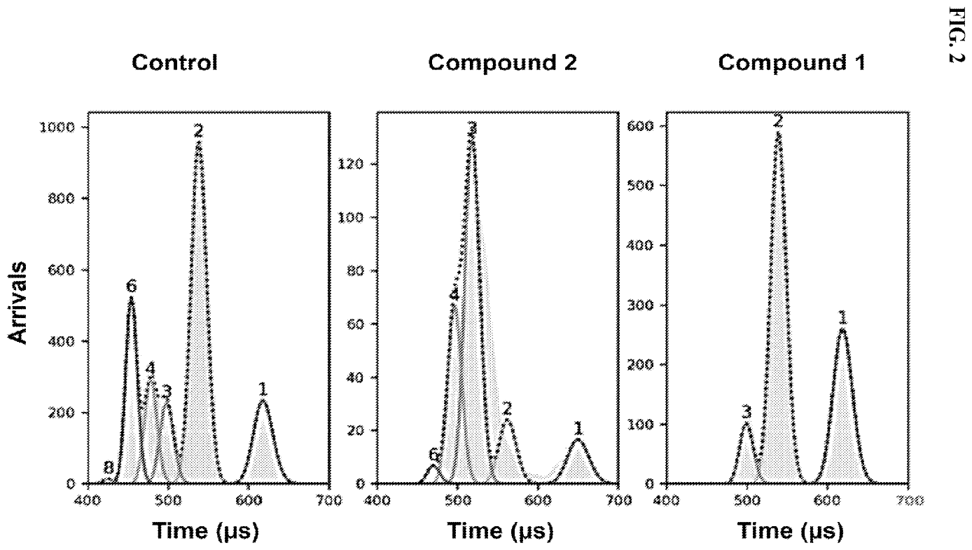

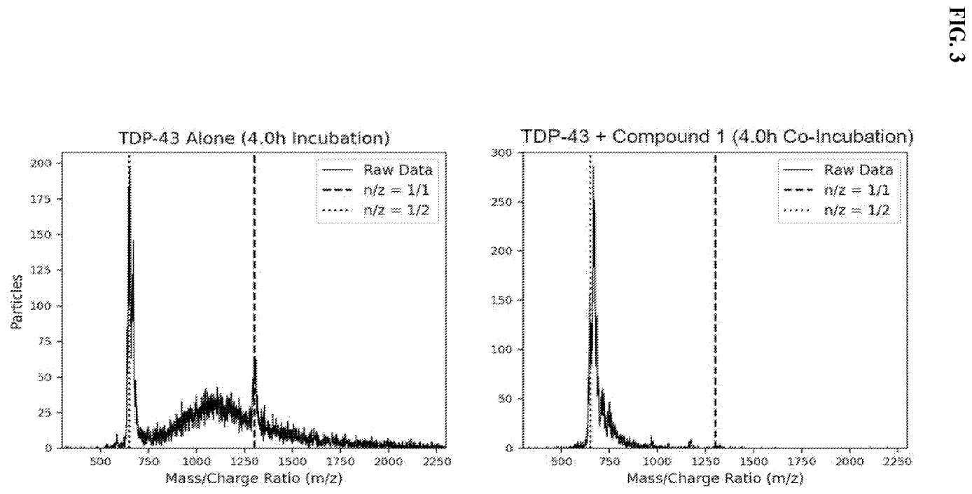

Atomic force microscopy (AFM) was utilized to study the effect of compound 1 on the formation of larger scale amyloid beta peptide 42 (Aβ42) protein aggregates and to determine the effect on pre-formed aggregates and fibrils. Amyloid beta peptide 42 protein aggregates are a major component of the amyloid plaques found in the brains of people with Alzheimer's disease. A 50 μL aliquot of 10 μM Aβ42 solution in 10 mM ammonium acetate was removed and drop-cast onto a mica disc, dried at ambient conditions in a desiccator, and imaged. It took approximately 5 min to prepare the peptide and for the disc to dry, making it the earliest time Aβ42 aggregation can be observed. After 5 min incubation, large globular aggregates formed but few fibrils were observed (FIG. 1A). After 60 min, there was a clear trend toward higher fibril content and larger oligomers. Thus, FIG. 1A represents a control, showing Aβ42 aggregation without the addition of any compound causing Aβ42 aggregation disruption.

Compound 1 was added to the same solution of pre-incubated Aβ42 in a 1:1 compound:Aβ42 ratio. The AFM image shown in FIG. 1B was taken at 60 minutes after compound 1 addition. FIG. 1B shows a dramatic reduction in Aβ42 protein aggregation after treatment with compound 1.

### Example 2: Ion-Mobility Mass Spectrometry (IM-MS)

One of the assays used to determine the effectiveness of the compounds is Ion Mobility Mass Spectroscopy, or IM-MS. In this assay, a protein solution was fed into the IM-MS tube. At the entrance to the tube, an electric field ionized the proteins, propelling them into the body of the tube, where a constant electric field propelled them forward. Upon reaching the end of the tube, the ionized proteins were counted by a particle detector, which can also measure the mass/charge ratio and flight time of each particle.

Methods: IM-MS was utilized to monitor the oligomerization of a peptide fragment of TAR DNA-binding protein 43 (TDP-43) in the presence of compounds 1 and 2 using methods as described in Laos et al ACS Chem. Neuro. 2019, 10 (9), 4112-4123; and Biochemistry 2019, 59 (4), 499-508. The accumulation of TDP-43 aggregates in the central nervous system is a common feature of many neurodegenerative diseases, such as amyotrophic lateral sclerosis, frontotemporal dementia, Alzheimer's disease, and limbic predominant age-related TDP-43 encephalopathy. The ability of IM-MS to separate TDP-43 aggregate species of a specific [m]z+ (m=mass, z=charge) that contains different [n]z+ (n=number of TDP-43 peptide molecules aggregated together) by measuring the arrival time distributions (ATDs) yields oligomeric distribution spectrographs.

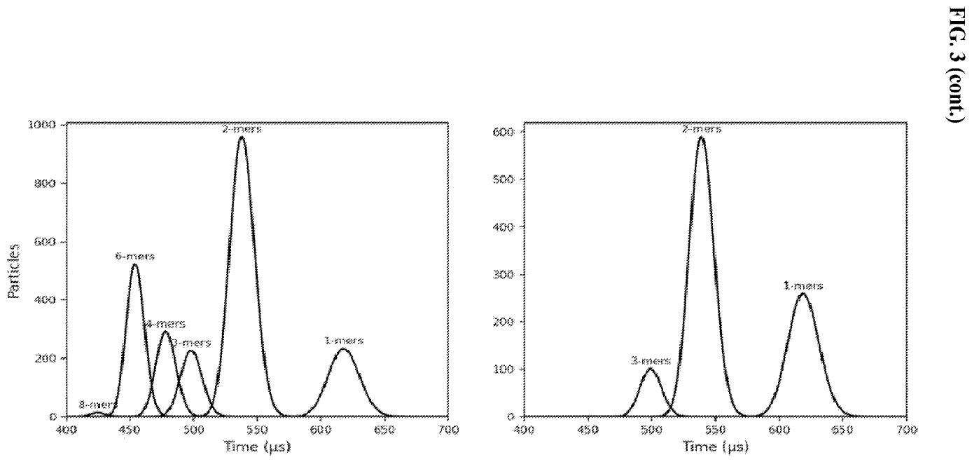

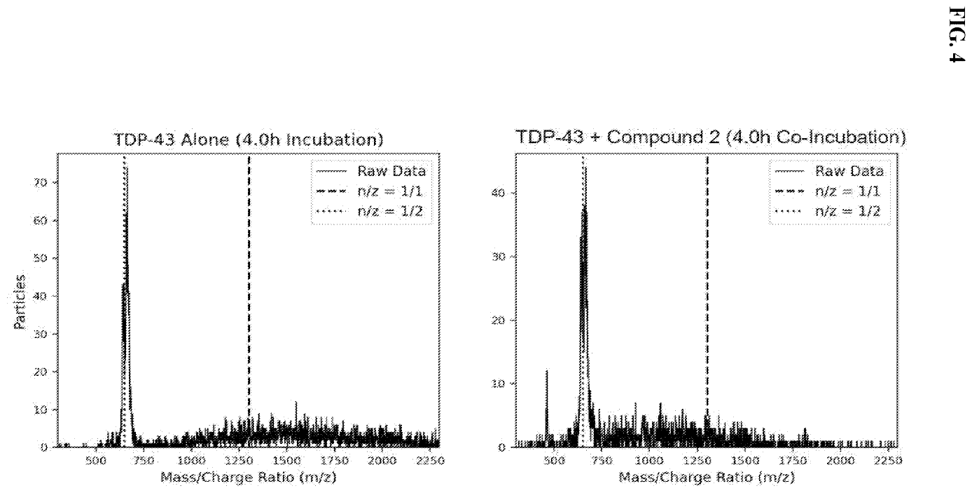

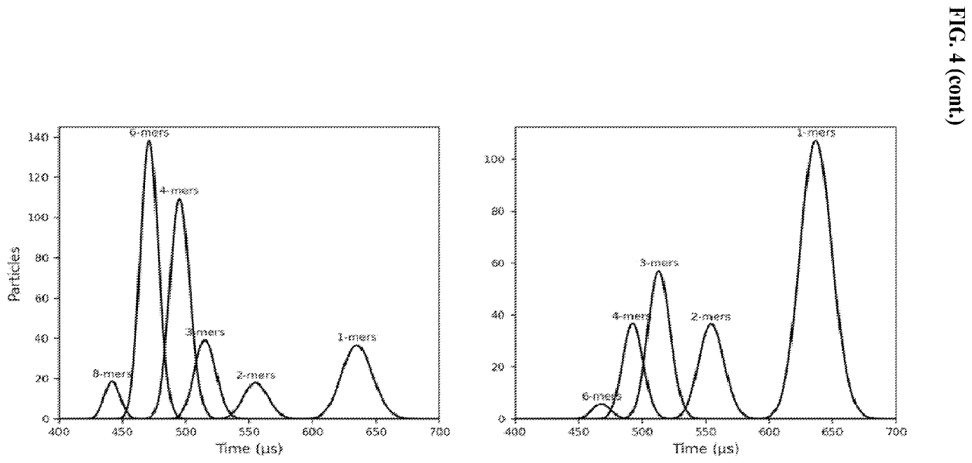

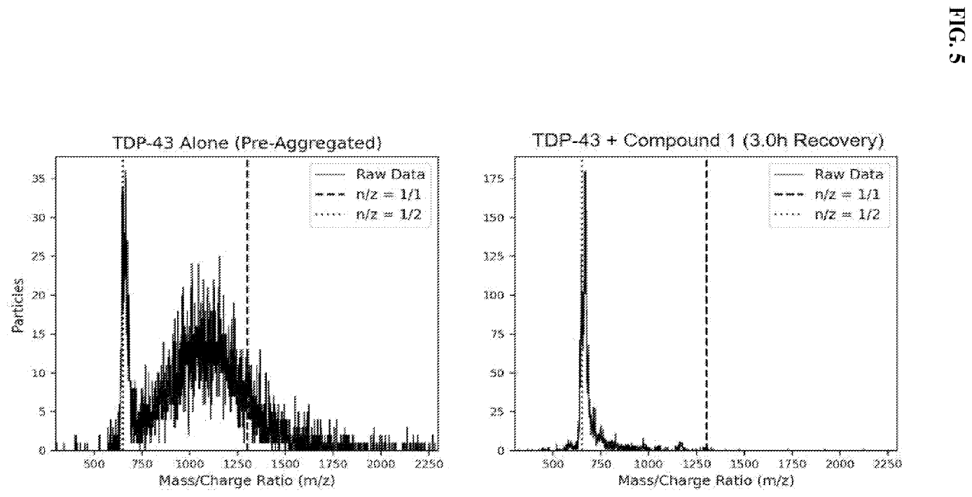

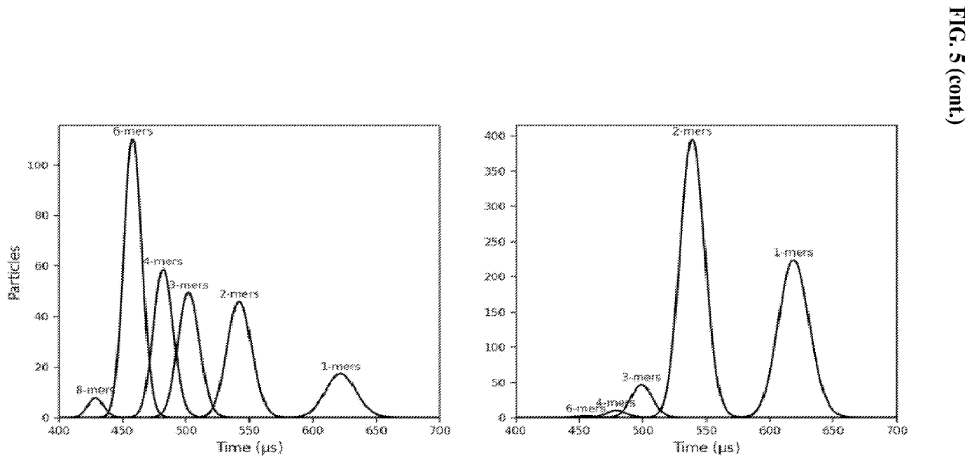

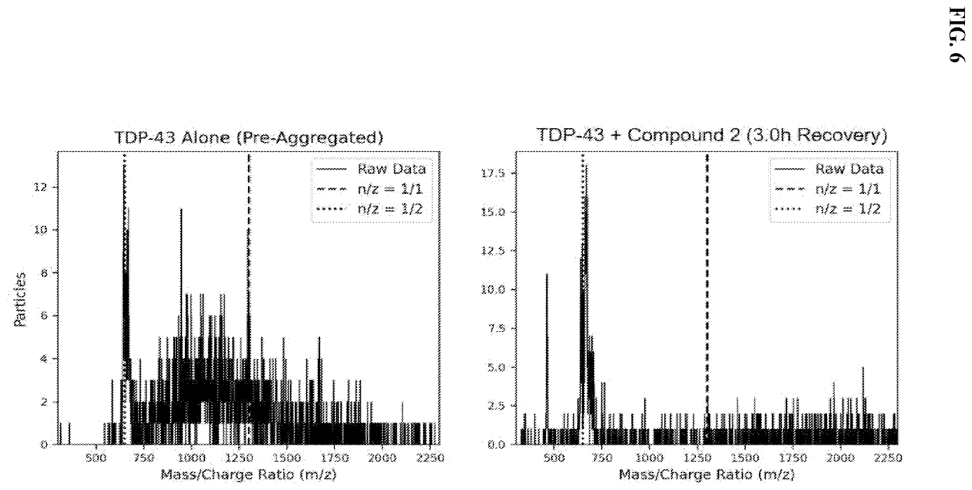

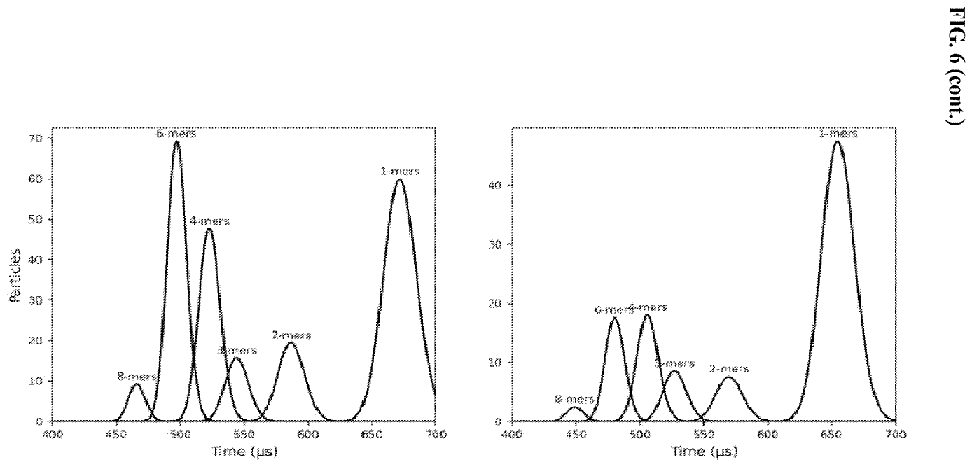

Using IM-MS, we observed the distribution of TDP-43 peptide aggregate sizes shown in FIG. 2. In FIG. 2, the number denoted at the top of each peak is the peptide aggregate size (e.g., 6=6 peptides aggregated together). The spectrograph designated as Control is the peptide form of TDP-43 without any protein aggregation disrupting compound added. Measurements were taken approximately 4 hours after addition of compound to aggregated peptide sample. The compound 1 and compound 2 spectrographs of FIG. 2 each show that the 6mer/hexamer peak present in the control is depleted after addition of compound 1 or 2. The 6mer is the toxic oligomer that propagates disease and neuronal degeneration. FIG. 2 also discloses that the addition of compound 1 or compound 2 decreased the number of higher-order TDP-43 oligomers and increased the number of TDP-43 monomers and dimers.

We conducted two types of experiments. The first was a co-incubation experiment, where we added the compounds to a fresh solution of monomeric protein, and allowed the protein to aggregate (or, ideally, not aggregate) in the presence of the compound. The second experiment was a recovery experiment, where we added our compounds to a solution of pre-aggregated protein. Co-incubation experiments allowed us to determine if the compounds can prevent initial aggregation, while recovery experiments allowed us to determine if the compounds can break up existing aggregates.

### Example 3: Neuronal Cell Toxicity Assay

Primary rat cortical and striatal neurons were prepared from foetuses' brain as previously described (Garcia et al., Diabetes, Obesity & Metabolism 2010, 12, 403-413; Roby et al., Food chemistry 2015, 171, 397-404; Colin et al., Biochimie 2016, 130, 178-187). Briefly, the embryos were extracted, brains were collected and dissected using a binocular magnifier. Embryonic cortical/striatal tissues were dissociated by a combination of enzymatic digestion and mechanical dissociation. The relative number of live cells in cell suspension was evaluated for each cortical and striatal neurons using Thoma cell counting chamber and Axio Scope A1 microscope (Carl Zeiss, Oberkochen, Germany). Dissociated neurons were plated (80,000 cells/well) in 48-well plates pre-coated with 15 μg/mL Poly-ornithine (Sigma), 1 μg/ml Laminin (Sigma). Cells were cultured in Neurobasal medium (Invitrogen, Paris, France) serum free of and supplemented with 2% (v/v) of B-27 Supplement (Invitrogen). Cultures were kept at 37° C. in a humidified 5% CO₂ atmosphere until 7 days in vitro.

To induce neuronal toxicity, neurotoxin (oligomeric forms of Aβ, Tau and alpha-synuclein at 0.1-5 μM) were used. Test compound solutions (1000-fold concentrated, working solutions) were prepared in the compound's vehicle. Working solutions were diluted into the culture medium. All of the culture medium was removed on in vitro day 4. Following this, 110 μl of compounds or compound's vehicle (1.1× concentrated solution) were added to cell culture wells. Then, 11 μl of neurotoxin or their vehicles (11× concentrated solution) were added. Cell viability was evaluated by a 3-(4,5-dimethylthiazol-2-yl)-2,5-diphenyl-2H-tetrazolium bromide (MTT) assay at 24-72 h post neurotoxin challenge. Briefly, cells were incubated at 37° C. for 1 h with MTT (0.5 mg/ml final concentration). Culture medium was removed and cells lysed in 100% DMSO. After complete solubilization of formazan, absorbance was recorded at 500 nm with a spectrophotometer (SpectraMax® i3x, Molecular Devices, San José, USA).

TABLE 1
Tau neuronal cell toxicity results. A higher value indicates greater
protection against the damaging effects of toxic tau protein aggregates
in cells. The positive control (no cell death) was set to 100% using
cells without tau oligomers. The negative control was set to 0%
using cells with tau oligomers and DMSO. The positive rescue control,
BDNF (Brain-Derived Neurotrophic Factor), resulted in 74% rescue.
Thus, the window of anti-aggregation activity observed for this
tau assay could be between 0 and 74% rescue.

| | % rescue at 10 μM compound |
|---|---|
| Compound ID | from toxic tau oligomers |
| Compound 2 | 33.7 |

TABLE 2
Alpha-synuclein neuronal cell toxicity results. A higher value
indicates greater protection against the damaging effects of toxic
alpha-synuclein protein aggregates in cells. The positive control
(no cell death) was set to 100% using cells without alpha-synuclein
oligomers. The negative control was set to 0% using cells with
alpha-synuclein oligomers and DMSO. The positive rescue control,
BDNF (Brain-Derived Neurotrophic Factor), resulted in 58% rescue.
Thus, the window of anti-aggregation activity observed for this
tau assay could be between 0 and 58% rescue.

| | % rescue at 10 μM comopund |
|---|---|
| Compound ID | from toxic alpha-synuclein oligomers |
| Compound 1 | 24 |

### Example 4: TDP-43 Cytoplasmic Aggregation Assay

Recombinant TDP-43-tGFP-U2OS cell line was thawed (2×10⁶ cells per T75) and was maintained in DMEM-F12 supplemented with 10% FBS at 37° C. in a humidified 5% CO₂ atmosphere. Cells were plated in 96-well plates with a density of 10,000 cells (+/−2000 cells) per well. Cells were incubated with the test compounds and isopropyl β-D-1-thiogalactopyranoside (IPTG) 1 mM to induce TDP43 expression for 24 hours. The assays were carried out in triplicate. Cells were treated with 125-250 μM of sodium arsenite for 120 min and TDP-43 punctae formation was quantified in both cell cytoplasm and cell nucleus, in living cells. The nuclei were stained using Hoechst (0.5 μg/ml) and the fluorescence was measured using a Cell Insight High-Content Bioimager from Thermo Fisher. To detect the Hoechst, the filters used were 380/10 and 460/10 nm for excitation and emission, respectively, and to detect the TDP-43 punctae, the filters were 488/20 and 520/20 nm, respectively. The images were obtained with an objective of 20×. Cell quantification was performed delimitating the region of interest of the nuclei and after quantification the average of each triplicate was performed. For granule quantification the spot detector application from Cell Software delimitated 2 regions of interest, the nuclei and cytosol. This software quantified the average number of granules per nuclei and the average number of granules per cytosol in each well.

TABLE 3
A lower score indicates higher potency and greater effectiveness
at removing toxic cytosolic aggregates.

| | TDP-43 |
|---|---|
| Compound ID | % cytosolic aggregates at 10 μM |
| Compound 1 | 34.5 |
| Compound 2 | 10.2 |

TABLE 4
A lower % remaining TDP-43 cytosolic aggregates
after 100 μM compound addition signals a
more potent compound in this cell-based assay.

| | % remaining TDP-43 cytosolic aggregates |
|---|---|
| Compound ID | after 100 μM compound addition |
| Compound 3 | >99.9 |
| Compound 4 | 59.1 |
| Compound 5 | 40.7 |
| Compound 6 | 93.7 |

### Example 5: Aβ42 Thioflavin T Assay

The effect of compounds on fibril formation by Aβ 1-42 was screened using a thioflavin T (ThT) fluorescence assay in a 96-well plate format. The monomeric form of Aβ 1-42 (rPeptide, A1170-025) film was diluted with 10 mM NaOH to form a 1 mg/ml stock solution. This stock solution was then further diluted with cell culture grade water (Cytiva, SH30529.01) and 10×PBS (VWR, 97063-660) to a working concentration of 100 μM in 1×PBS on ice. Compounds were initially dissolved in 100% DMSO (MilliporeSigma, D2650) at room temperature to make a 10 mM stock and stored at −20° C. prior to the experiment. On the day of the experiment, the stock solution was diluted with 1×PBS to a concentration of 500 μM, containing 5% DMSO. The reaction mixture, containing 10 μM ThT (rPeptide, A1170-025), 5 μM Aβ 1-42, and 1×PBS was prepared in 15 ml Falcon centrifuge tubes (Corning, 352070) and kept on ice during preparation. The reaction mixture was then dispensed into multiple wells of a 96-well half-area, low-binding polyethylene non-treated plate (Corning, 3880) with a clear bottom, at 90 μL per well. To each well, 10 μL of 500 μM compound stock was added, with three replicates per compound. To account for the presence of DMSO, 5% DMSO in 1×PBS was used as the vehicle control. The positive control consisted of 10 μM ThT, 5 μM Aβ 1-42, and 0.5% DMSO in 1×PBS, while the negative control consisted of 10 μM ThT and 0.5% DMSO in 1×PBS. The plates were sealed with polyester sealing tape (VWR, 89134-430) to prevent evaporation and then placed in a plate reader (Molecular Devices SpectraMax ID5) at 37° C. without agitation. ThT fluorescence was measured through the bottom of the plate using 440- and 480-nm excitation and emission filters for 15 hours at 0.5-hour intervals. Each well was scanned at 5 points with a 1.25 mm distance between points. The relative fluorescence unit (RFU) at 15 hours was used to rank the percent of aggregation for all tested compounds and controls.

TABLE 5
ThT results. A lower score means more potent anti-aggregation properties
for all aggregate size ranges (not just a specific size range).
The negative aggregation control was set to zero using no Aβ. The
positive aggregation control was set to 100% using Aβ +
DMSO. Averaged negative control values were subtracted from all
sample values and then the % aggregation of samples was calculated
as percentage of average aggregation of the positive control.

| | Aβ42 |
|---|---|
| Compound ID | % aggregation at 50 μM |
| Compound 1 | 60.9 |
| Compound 2 | 73.5 |
| Compound 3 | >99.9 |
| Compound 4 | 74.6 |
| Compound 5 | 58.4 |
| Compound 6 | 59.7 |

### Example 6: TDP-43 Thioflavin T Assay

The effect of compounds on fibril formation by TDP-43 peptide (amino acids 307-319) was screened using a thioflavin T (ThT) fluorescence assay in a 96-well plate format. The monomeric form of TDP-43 307-319 was stored in HFIP at −20° C. in 32 μL vials and then thawed on ice. After thawing a stock aliquot on ice, 16 μL of HFIP and 432 μL 100 mM ammonium acetate was added. This mix was pipetted three times. Three such prepared aliquots were combined to create a master mix. 400 μM of ThT stock was prepared by dissolving 31.8 mg ThT in 10 mL Milli-Q water to make 10 mmM stock. The 10 mM ThT stock was then diluted to 400 μM with Milli-Q water and stored at −20° C. until use. 20 μL aliquots of compounds were used as initial starting material. 98 μL of master mix was then added to each well of 96-well plate, and then 2 μL of compounds was added to each well testing a compound. The plate was spun at 4700×rpm to remove bubbles at 4° C., and then the plate measurements were collected. ThT fluorescence was measured through the bottom of the plate using 440- and 480-nm excitation and emission filters for 15 hours at 0.5-hour intervals. Each well was scanned at 5 points with a 1.25 mm distance between points. The relative fluorescence unit (RFU) at 15 hours was used to rank the percent of aggregation for all tested compounds.

TABLE 6
ThT results. A lower score means more potent anti-aggregation
properties for all aggregate size ranges (not just a specific
size range). The negative aggregation control was set to
zero using DMSO and no TDP-43. The positive aggregation control
was set to 100% using TDP-43 + DMSO. Averaged negative
control values were subtracted from all sample values and
then the % aggregation of samples was calculated as percentage
of average aggregation of the positive control.

| | % aggregation TDP-43 |
|---|---|
| Compound ID | ThT at 50 μM TDP-43 |
| Compound 1 | 77.4 |
| Compound 2 | 80.7 |
| Compound 3 | 78.9 |
| Compound 5 | 64.6 |
| Compound 6 | 73.1 |
| Compound 12 | 43.5 |
| Compound 13 | 48.8 |
| Compound 14 | 48.6 |
| Compound 15 | 47.2 |

### Example 7: Alpha-Synuclein Thioflavin T Assay

Recombinant alpha-synuclein monomeric protein was procured from Proteos (catalog no. RP-003, 10 mg/ml or 700 μM concentration). Prior to aggregation, the protein was centrifuged at 10,000 rpm for 5 minutes to remove any pre-existing aggregates. The stock solution was then diluted to 50 μM concentration in filtered 1×PBS and allowed to aggregate with continuous shaking at 1000 rpm on a thermomixer for 96 hours at 37° C. After 96 hours, the ThT fluorescence was checked for the sample to confirm aggregate formation using ThT, a well-known amyloid marker dye. The ThT active aggregates were then used for high-throughput small molecule inhibitor screening. For screening small molecule disaggregators, 10 mM DMSO stock solutions of small molecules were diluted to 250 μM in the reaction mix containing 20 μM ThT and aggregates formed by 50 μM alpha-synuclein. As a positive/vehicle control, 100% DMSO stock was diluted to 2.5% to match the DMSO in the small molecule conditions. ThT fluorescence assay was performed for 24 hours with 3 replicates per sample in a 384 well plate (Corning low volume black/clear flat bottom) on a microplate reader (Molecular Devices SpectraMax iD5 or BioTek Agilent Synergy Neo2) using excitation wavelength of 440 nm and emission wavelength of 490 nm. The temperature was maintained at 37° C. and the plate was continuously shaken throughout the measurement. All data were plotted and analyzed in GraphPad Prism.

TABLE 7
ThT results. A lower score means more potent anti-aggregation
properties for all aggregate size ranges (not just a specific
size range). The negative aggregation control was set to zero
using DMSO and no alpha synuclein. The positive aggregation
control was set to 100% using alpha synuclein + DMSO. Averaged
negative control values were subtracted from all sample values
and then the % aggregation of samples was calculated as percentage
of average aggregation of the positive control.

| | % alpha synuclein aggregation |
|---|---|
| Compound ID | as measured on ThT |
| Compound 1 | 77.8 |
| Compound 2 | 95.6 |
| Compound 3 | 82.8 |
| Compound 4 | >99.9 |
| Compound 5 | 99 |
| Compound 6 | >99.9 |

### Example 8: Experimental Procedures

General Information: Reagents and solvents obtained from commercial suppliers were used without purification or drying unless otherwise noted. ¹H NMR were recorded using a Bruker mass spectrometer run at 400 MHz & 500 MHz using tetramethylsilane (TMS) as an internal standard. LCMS analysis was performed on Waters UPLC with SQD2 mass detector (Single quadrupole), HPLC analyses were performed on an Acquity UPLC H-CLASS with WATERS.

### Experimental Procedures for Compound 3:

### Synthesis of Compound Int-2:

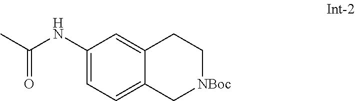 [SMILES: CC(=O)Nc1ccc2c(c1)CCN(C(=O)OC(C)(C)C)C2]

tert-Butyl 6-acetamido-3.4-dihydroisoquinoline-2(1H)-carboxylate: The mixture of tert-butyl 6-amino-3,4-dihydroisoquinoline-2(1H)-carboxylate (0.5 g, 2.013 mmol, 1.0 eq.) and acetic anhydride (0.61 g, 6.012 mmol, 3.0 eq.) was stirred at 25° C. for 16 h. The progress of the reaction was monitored by TLC and LCMS. After completion of the reaction, the reaction mixture was poured into ice-water (20 mL) and extracted with dichloromethane twice. The combined organic layer was dried over by anhydrous Na₂SO₄, filtered and concentrated under reduced pressure to give crude compound. to afford the desired compound Int-2 (0.4 g, Y: 68%) as an off white solid. ¹H NMR (400 MHz, DMSO-d₆) δ 9.84 (s, 1H), 7.42 (s, 1H), 7.32 (d, J=8.0 Hz, 1H), 7.05 (d, J=8.4 Hz, 1H), 4.41 (s, 2H), 3.52 (t, J=5.6 Hz 2H), 2.72 (t, J=5.6 Hz 2H), 2.01 (s, 3H), 1.42 (s, 9H). LC-MS (M-BOC+H): 191.4.

### Synthesis of Compound Int-3:

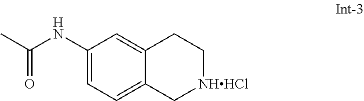

N-(1,2,3,4-Tetrahydroisoquinolin-6-yl) acetamide hydrochloride: To a stirred mixture of tert-butyl 6-acetamido-3,4-dihydroisoquinoline-2(1H)-carboxylate (0.4 g, 1.374 mmol, 1.0 eq.) in dichloromethane (10 mL) was added 4M HCl in dioxane (5 mL) at 0° C. and the reaction mixture was stirred at 25° C. for 1 h. The progress of the reaction was monitored by TLC and LCMS. After completion of the reaction, the reaction mixture was evaporated under reduced pressure under nitrogen atmosphere and washed with di ethyl ether, dried under reduced pressure to afford the desired compound Int-3 (0.25 g, Y: 96%) as an off white solid. LC-MS (M+H): 191.4.

### Synthesis of Compound 3:

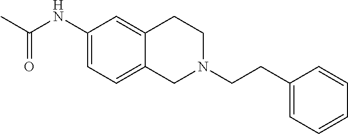 [SMILES: CC(=O)Nc1ccc2c(c1)CCN(CCc1ccccc1)C2]

N-(2-Phenethyl-1,2,3,4-tetrahydroisoquinolin-6-yl) acetamide: To a stirred mixture of N-(1,2,3,4-tetrahydroisoquinolin-6-yl) acetamide hydrochloride (0.15 g, 0.663 mmol, 1.0 eq.) in dimethylformamide (2 mL) were added DIPEA (0.256 g, 1.991 mmol, 3.0 eq.) and (2-bromoethyl)benzene (0.147 g, 0.7.964 mmol, 1.2 eq.) at 0° C. and the reaction mixture was stirred at 70° C. for 16 h. The progress of the reaction was monitored by TLC and LCMS. After completion of the reaction, the reaction mixture was quenched with ice cold water and extracted with ethyl acetate twice. The combined organic layers were dried over by anhydrous Na₂SO₄, filtered and concentrated under reduced pressure to give crude compound. The crude was purified by prep HPLC. mobile phase: A 10 mm NH₄HCO₃ in water. Column YMC ACTUS (150*12) mm3 um. the fractions were lyophilized to afford the desired compound 3 (0.09 g, Y: 46%) as an off white solid. ¹H NMR (400 MHz, DMSO-d₆) δ 9.78 (s, 1H), 7.35 (s, 1H), 7.29-7.26 (m, 5H), 7.20-7.16 (m, 1H), 6.96 (d, J=8.4 Hz, 1H), 3.56 (s, 2H), 2.84-2.65 (m, 8H), 2.00 (s, 3H). LC-MS (M+H): 295.24.

### Experimental Procedures for Compound 4:

### Synthesis of Compound Int-2:

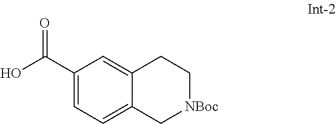 [SMILES: CC(C)(C)OC(=O)N1CCc2cc(C(=O)O)ccc2C1]

2-(tert-Butoxycarbonyl)-1,2,3,4-tetrahydroisoquinoline-6-carboxylic acid: To a stirred mixture of tert-butyl 6-bromo-3,4-dihydroisoquinoline-2(1H)-carboxylate (0.5 g, 1.607 mmol, 1.0 eq.) in 1,4-dioxane (3 mL) and water (3 mL) under inert atmosphere at room temperature was added NaOAc (0.263 g, 3.214 mmol, 2.0 eq.) and degassed for 5 min. This was followed by the addition of Pd(dppf)Cl₂ (0.129 g, 0.160 mmol, 0.1 eq.) and Co₂(CO)₈ (0.138 g, 0.964 mmol, 0.6 eq.) at room temperature and the reaction mixture was stirred at 70° C. for 16 h. The progress of the reaction was monitored by TLC and LCMS. After completion of the reaction, the reaction mixture was poured into ice-water and acidified with saturated citric acid solution and extracted with ethyl acetate twice. The combined organic layers were dried over by anhydrous Na₂SO₄, filtered and concentrated under reduced pressure to give crude compound. The crude material was triturated with diethyl ether, offered compound Int-2 (0.3 g, Y: 71%) as an off white solid compound. ¹H NMR (400 MHz, DMSO-d₆) δ 12.85 (br. s, 1H), 7.73-7.72 (m, 2H), 7.27 (d, J=8.4 Hz, 1H), 4.55 (s, 2H), 3.56 (t, J=5.8 Hz, 2H), 2.83 (t, J=5.8 Hz, 2H), 1.43 (s, 9H). LC-MS (M−H): 276.18.

### Synthesis of Compound Int-3:

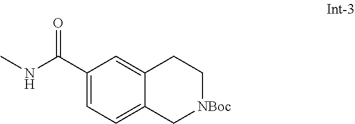 [SMILES: CNC(=O)c1ccc2c(c1)CCN(C(=O)OC(C)(C)C)C2]

tert-Butyl 6-(methylcarbamoyl)-3,4-dihydroisoquinoline-2(1H)-carboxylate: To a stirred mixture of 2-(tert-butoxy carbonyl)-1,2,3,4-tetrahydroisoquinoline-6-carboxylic acid (0.30 g, 1.083 mmol, 1.0 eq.) in dimethylformamide (6 mL) was added DIPEA (0.47 mL, 2.707 mmol, 2.5 eq.), HATU (0.61 g, 1.624 mmol, 1.5 eq.) and 2M methyl amine solution in THF (0.81 mL, 1.624 mmol, 1.5 eq.) at 0° C. and the reaction mixture was stirred at 25° C. for 2 h. The progress of the reaction was monitored by TLC and LCMS. After completion of the reaction, the reaction mixture was quenched with ice cold water and extracted with ethyl acetate twice. The combined organic layers were dried over by anhydrous Na₂SO₄, filtered and concentrated under reduced pressure to give crude compound. The crude was purified by column chromatography using 100-200 silica-gel mesh, eluted with 80% ethyl acetate/pet ether to afford the desired compound Int-3 (0.2 g, Y: 63%) as white solid compound. ¹H NMR (500 MHz, CDCl₃) δ 7.57-7.53 (m, 2H), 7.15 (d, J=8.0 Hz, 1H), 6.13 (br. s, 1H), 4.59 (s, 2H), 3.65 (t, J=5.8 Hz 2H), 3.10 (s, 3H), 2.86 (t, J=5.8 Hz 2H), 1.49 (s, 9H). LC-MS (M+H): 291.25.

### Synthesis of Compound Int-4:

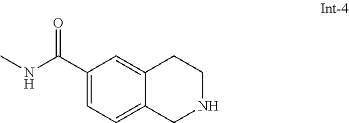

N-Methyl-1,2,3,4-tetrahydroisoquinoline-6-carboxamide: To a stirred mixture of tert-butyl 6-(methylcarbamoyl)-3,4-dihydroisoquinoline-2(1H)-carboxylate (0.2 g, 0.689 mmol, 1.0 eq.) in dichloromethane (5 mL) was added 4M HCl in diethyl ether (0.86 mL) at 0° C. and the reaction mixture was stirred at 25° C. for 4 h. the progress of the reaction was monitored by TLC. After completion of the reaction, the reaction mixture was evaporated under reduced pressure under nitrogen atmosphere and washed with di ethyl ether, dried under reduced pressure to afford the desired compound Int-4 (0.13 g, Y: 100%) as an off white solid. The crude was used for next step based on TLC.

### Synthesis of Compound 4:

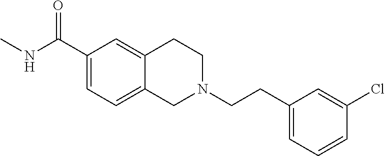 [SMILES: CNC(=O)c1ccc2c(c1)CCN(CCc1cccc(Cl)c1)C2]

2-(3-chlorophenethyl)-N-methyl-1.2.3.4-tetrahydroisoquinoline-6-carboxamide: To a stirred mixture of N-methyl-1,2,3,4-tetrahydroisoquinoline-6-carboxamide (0.15 g, 0.785 mmol, 1.0 eq.) in dimethylformamide (5 mL) was added N,N-diisopropylethylamine (0.255 g, 2.356 mmol, 3.0 eq.) and 1-(2-bromoethyl)-3-chlorobenzene (0.204 g, 0.942 mmol, 1.2 eq.) at 0° C. and the reaction mixture was stirred at 70° C. for 16 h. The progress of the reaction was monitored by TLC and LCMS. After completion of the reaction, the reaction mixture was quenched with ice cold water and extracted with ethyl acetate twice. The combined organic layers were dried over by anhydrous Na₂SO₄, filtered and concentrated under reduced pressure to give crude compound. The crude was purified by prep HPLC. mobile phase: A 10 mm NH₄HCO₃ in water. Column YMC ACTUS (150*12) mm3 um. the fractions were lyophilized to afford the desired compound 4 (0.065 g, Y: 25%) as an off white solid. ¹H NMR (400 MHz, DMSO-d₆) δ 8.32-8.31 (m, 1H), 7.57-7.55 (m, 2H), 7.36 (t, J=1.6 Hz, 1H), 7.33-7.30 (m, 1H), 7.25-7.23 (m, 2H), 7.12 (d, J=8.0 Hz, 1H), 3.65 (s, 2H), 2.87-2.69 (m, 11H). LC-MS (M+H): 329.28.

### Synthetic Scheme and Experimental Procedure for Compound 5:

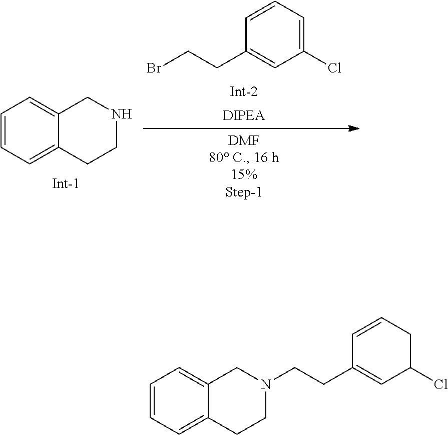 [SMILES: Clc1cccc(CCBr)c1 ; c1ccc2c(c1)CCNC2 ; ClC1C=C(CCN2CCc3ccccc3C2)C=CC1]

2-(3-chlorophenethyl)-1,2,3,4-tetrahydroisoquinoline: To a stirred mixture of 1,2,3,4-tetrahydroisoquinoline (0.2 g, 1.503 mmol, 1.0 eq.) and 1-(2-bromoethyl)-3-chlorobenzene (0.39 g, 1.804 mmol, 1.0 eq.) in dimethylformamide (4 mL) was added N,N-diisopropylethylamine (0.75 mL, 4.509 mmol, 3.0 eq.) at 0° C. and the reaction mixture was stirred at 80° C. for 16 h. The progress of the reaction was monitored by TLC and LCMS. After completion of the reaction, the reaction mixture was quenched with ice cold water, and extracted with dichloromethane twice. The combined organic layers were dried over by anhydrous Na₂SO₄, filtered and concentrated under reduced pressure to give crude compound. The crude was purified by prep HPLC. mobile phase: A 10 mm NH₄HCO₃ in water. Column YMC ACTUS (150*12) mm3 um. the fractions were lyophilized to afford the desired compound 5 (0.019 g, Y: 15%) as colourless gum. ¹H NMR (400 MHz, DMSO-d₆) δ 7.35 (s, 1H), 7.30 (t, J=8.6 Hz, 1H), 7.23 (d, J=8.0 Hz, 2H), 7.11-7.03 (m, 4H), 3.61 (s, 2H), 2.86-2.82 (m, 2H), 2.80-2.77 (m, 2H), 2.72-2.67 (m, 4H). LC-MS (M+H): 272.18.

### Experimental Procedures for Compound 6:

### Synthesis of Compound Int-2:

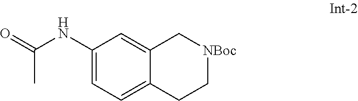 [SMILES: CC(=O)Nc1ccc2c(c1)CN(C(=O)OC(C)(C)C)CC2]

tert-Butyl 7-acetamido-3,4-dihydroisoquinoline-2(1H)-carboxylate: The mixture of tert-butyl 7-amino-3,4-dihydroisoquinoline-2(1H)-carboxylate (0.5 g, 2.013 mmol, 1.0 eq.) and acetic anhydride (0.61 g, 6.012 mmol, 3.0 eq.) was stirred at 25° C. for 16 h. The progress of the reaction was monitored by TLC and LCMS. After completion of the reaction, the reaction mixture was poured into ice-water (20 mL), and extracted with dichloromethane twice. The combined organic layer was dried over by anhydrous Na₂SO₄, filtered and concentrated under reduced pressure to give the desired compound Int-2 (0.4 g, Y: 68%) as an off white solid. ¹H NMR (400 MHz, CDCl₃) δ 7.48 (br. s, 1H), 7.29 (s, 1H), 7.06 (d, J=7.6 Hz, 2H), 4.54 (s, 2H), 3.64-3.60 (m, 2H), 2.79-2.76 (m, 2H), 2.16 (s, 3H), 1.49 (s, 9H). LC-MS (M-BOC+H): 191.4.

### Synthesis of Compound Int-3:

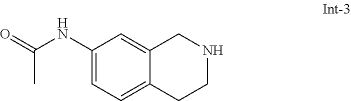

N-(1,2,3,4-Tetrahydroisoquinolin-7-yl) acetamide: To a stirred mixture of tert-butyl 7-acetamido-3,4-dihydroisoquinoline-2(1H)-carboxylate (0.4 g, 1.374 mmol, 1.0 eq.) in dichloromethane (10 mL), was added 4M HCl in dioxane (2 mL) at 0° C. and the reaction mixture was stirred at 25° C. for 1 h. The progress of the reaction was monitored by TLC and LCMS. After completion of the reaction, the reaction mixture was evaporated under reduced pressure and washed with di ethyl ether, dried under reduced pressure to afford the desired compound Int-3 (0.25 g, Y: 96%) as an off white solid. ¹H NMR (400 MHz, DMSO-d₆) δ 10.05 (s, 1H), 9.44 (s, 1H), 7.51 (s, 1H), 7.38 (dd, J=2.0, 8.4 Hz, 1H), 7.13 (d, J=8.4 Hz 1H), 4.21 (t, J=4.4 Hz 2H), 3.33-3.31 (m, 2H), 2.93 (t, J=6.2 Hz 2H), 2.03 (s, 3H). LC-MS (M+H): 191.4.

### Synthesis of Compound 6:

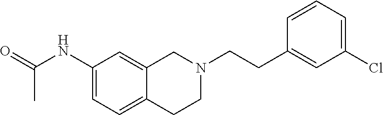 [SMILES: CC(=O)Nc1ccc2c(c1)CN(CCc1cccc(Cl)c1)CC2]

N-(2-(3-Chlorophenethyl)-1,2,3,4-tetrahydroisoquinolin-7-yl) acetamide: To a stirred mixture of N-(1,2,3,4-tetrahydroisoquinolin-7-yl) acetamide (0.24 g, 1.263 mmol, 1.0 eq.) in dimethylformamide (4 mL) was added N,N-diisopropylethylamine (0.5 mL, 2.526 mmol, 2.0 eq.) and 1-(2-bromoethyl)-3-chlorobenzene (0.33 g, 1.515 mmol, 1.2 eq.) at 0° C. and the reaction mixture was stirred at 80° C. for 16 h. The progress of the reaction was monitored by TLC and LCMS. After completion of the reaction, the reaction mixture was quenched with ice cold water and extracted with ethyl acetate twice. The combined organic layers were dried over by anhydrous Na₂SO₄, filtered and concentrated under reduced pressure to give crude compound. The crude was purified by prep HPLC. mobile phase: A 10 mm NH₄HCO₃ in water. Column YMC ACTUS (150*12) mm3 um. the fractions were lyophilized to afford the desired compound 6 (0.064 g, Y: 15%) as a brown solid. ¹H NMR (400 MHz, DMSO-d₆) δ 9.78 (s, 1H), 7.35-7.30 (m, 3H), 7.26-7.22 (m, 3H), 6.99 (d, J=8.4 Hz, 1H), 3.56 (s, 2H), 2.85 (t, J=8.4 Hz, 2H), 2.70-2.66 (m, 6H), 2.00 (s, 3H). LC-MS (M+H): 329.25.

### Synthetic Scheme and Experimental Procedure for Compound 7:

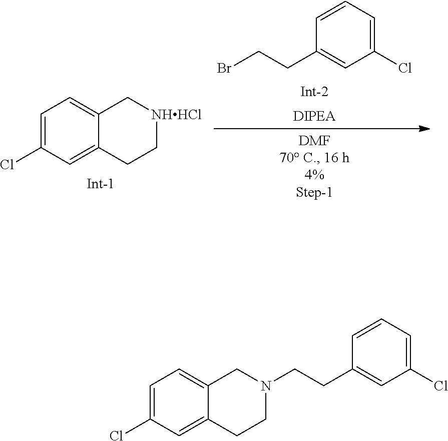

6-Chloro-2-(3-chlorophenethyl)-1,2,3,4-tetrahydroisoquinoline: To a stirred solution of 6-chloro-1,2,3,4-tetrahydroisoquinoline hydrochloride (0.2 g, 0.98 mmol, 1.0 eq.) in N,N-dimethylformamide (5 mL) were added N. N-diisopropylethylamine (0.51 mL, 2.94 mmol, 3.0 eq.) and 1-(2-bromoethyl)-3-chlorobenzene (0.214 g, 0.98 mmol, 1.0 eq.) at room temperature and the reaction mixture was stirred at 70° C. for 16 h. The progress of the reaction was monitored by TLC and LCMS. After completion of the reaction, the reaction mixture was poured into ice-water and extracted with ethyl acetate twice. The combined organic layer was washed with water, brine, dried over by anhydrous Na₂SO₄, filtered and concentrated under reduced pressure to give the crude compound. The crude was purified by prep HPLC, Mobile Phase: A-10 mM Ammonium bicarbonate in Water: B-Acetonitrile Column: X-Terra 19×50 mm, 5 μm Gradient: Time/% B 0/40; 11/95; 11.1/100; 15/100; 15.1/40; 17/40 Flow Rate: 15 ml/min. The fractions were lyophilized to afford the desired compound 7 (0.012 g, Y: 4%) as an off-white solid. ¹H NMR (400 MHz, DMSO-d₆) δ 7.35-7.34 (m, 1H), 7.32-7.29 (m, 1H), 7.25-7.22 (m, 2H), 7.17-7.14 (m, 2H), 7.08 (d, J=8.0 Hz, 1H), 3.59 (s, 2H), 2.86-2.78 (m, 4H), 2.72-2.67 (m, 4H). LC-MS (M+H): 306.31.

### Experimental Procedures for Compound 8:

### Synthesis of Compound Int-2:

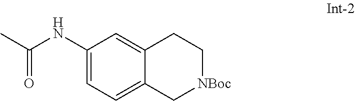 [SMILES: CC(=O)Nc1ccc2c(c1)CCN(C(=O)OC(C)(C)C)C2]

tert-Butyl 6-acetamido-3,4-dihydroisoquinoline-2(1H)-carboxylate: The mixture of tert-butyl 6-amino-3,4-dihydroisoquinoline-2(1H)-carboxylate (0.5 g, 2.02 mmol, 1.0 eq.) and acetic anhydride (0.6 mL, 6.05 mmol, 3.0 eq.) was stirred at the room temperature for 16 h. The progress of the reaction was monitored by TLC and LCMS. After completion of the reaction, the reaction mixture was diluted with water and extracted with ethyl acetate twice. The combined organic layer was washed with water, brine, dried over by anhydrous Na₂SO₄, filtered and concentrated under reduced pressure to give Int-2 (0.450 g, Y: 77%) as colourless gum. ¹H NMR (500 MHz, CDCl₃) δ 7.47-7.27 (m, 2H), 7.13 (br. s, 1H), 7.03 (d, J=1.0 Hz, 1H), 4.52 (s, 2H), 3.64-3.61 (m, 2H), 2.84-2.79 (m, 2H), 2.22 (s, 9H), 2.09 (s, 3H). LC-MS (M-BOC+H): 191.23.

### Synthesis of Compound Int-3:

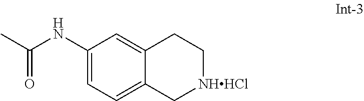

N-(1,2,3,4-Tetrahydroisoquinolin-6-yl) acetamide hydrochloride: To a stirred mixture of Int-2 (0.450 g, 1.55 mmol, 1.0 eq.) in dichloromethane (10 mL) was added 4 M HCl in 1,4-dioxane (4 mL) at room temperature and the reaction mixture was stirred at the same temperature for 12 h. The progress of the reaction was monitored by TLC and LCMS. After completion of the reaction, the reaction mixture was concentrated under reduced pressure to give Int-3 (0.2 g, crude) as an off-white solid. LC-MS (M+H): 191.17.

### Synthesis of Compound 8:

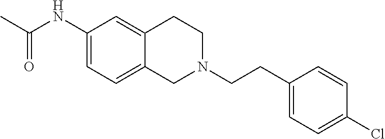 [SMILES: CC(=O)Nc1ccc2c(c1)CCN(CCc1ccc(Cl)cc1)C2]

N-(2-(4-Chlorophenethyl)-1,2,3,4-tetrahydroisoquinolin-6-yl) acetamide: To a stirred solution of Int-3 (0.2 g, 0.88 mmol, 1.0 eq.) in N,N-dimethylformamide (5 mL) was added N,N-diisopropylethylamine (0.46 mL, 2.65 mmol, 3.0 eq.) and 1-(2-bromoethyl)-4-chlorobenzene (0.232 g, 1.06 mmol, 1.2 eq.) at 0° C. and the reaction mixture was stirred at 70° C. for 16 h. The progress of the reaction was monitored by TLC and LCMS. After completion of the reaction, the reaction mixture was poured into ice-water and extracted with ethyl acetate twice. The combined organic layer was washed with water, brine, dried over by anhydrous Na₂SO₄, filtered and concentrated under reduced pressure to give the crude compound. The crude compound was purified by prep. HPLC, Mobile Phase: A-10 mM Ammonium bicarbonate in Water: B-Acetonitrile Column: YMC Trait C18 (150*25) mm 7 μm, Gradient: Time/% B 0/30; 11/95; 11.1/100; 13/100; 13.1/30; 16/30 Flow Rate: 20 ml/min to afford compound 8 (0.098 g, Y: 34%) as an off-white solid. ¹H NMR (400 MHz, DMSO-d₆) δ 9.78 (s, 1H), 7.35-7.25 (m, 6H), 6.95 (d, J=8.4 Hz, 1H), 3.54 (s, 2H), 2.82 (t, J=7.4 Hz, 2H), 2.76-2.73 (m, 2H), 2.69-2.64 (m, 4H), 2.00 (s, 3H). LC-MS (M+H): 329.35.

### Synthetic Scheme and Experimental Procedure for Compound 9:

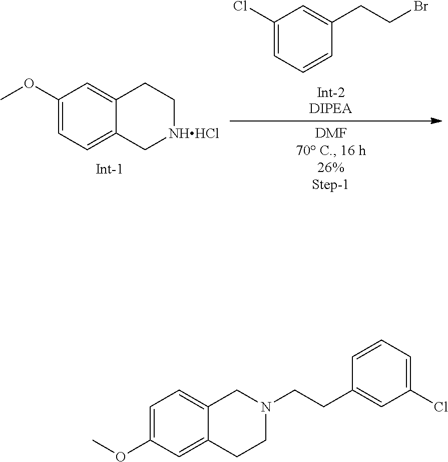

2-(3-Chlorophenethyl)-6-methoxy-1,2,3,4-tetrahydroisoquinoline: To a stirred solution of 6-methoxy-1,2,3,4-tetrahydroisoquinoline hydrochloride (0.2 g, 1.00 mmol, 1.0 eq.) in N,N-dimethylformamide (5 mL) were added N,N-diisopropylethylamine (0.52 mL, 3.00 mmol, 3.0 eq.) and 1-(2-bromoethyl)-3-chlorobenzene (0.264 g, 1.2 mmol, 1.2 eq.) at room temperature and the reaction mixture was stirred at 70° C. for 16 h. The progress of the reaction was monitored by TLC and LCMS. After completion of the reaction, the reaction mixture was poured into ice-water and extracted with ethyl acetate twice. The combined organic layer was dried over by anhydrous Na₂SO₄, filtered and concentrated under reduced pressure to give compound. The crude compound was purified by prep. HPLC, Mobile Phase: A—10 mM Ammonium bicarbonate in Water: B—Acetonitrile Column: Kromasil C18 (150*25) mm 7 μm gradient: Time/% B 0/40; 5/67; 12/67; 12.1/100; 13/100; 13.1/40; 16/40 Flow Rate: 20 ml/min to afford compound 9 (79 mg, Y: 26%) as an off-white solid. ¹H NMR (400 MHz, DMSO-d₆) δ 7.35-7.33 (m, 1H), 7.32-7.28 (m, 1H), 7.23 (d, J=7.2 Hz, 2H), 6.95 (d, J=8.4 Hz, 1H), 6.70-6.65 (m, 2H), 3.70 (s, 3H), 3.53 (s, 2H), 2.83 (t, J=7.4 Hz, 2H), 2.76 (t, J=5.4 Hz, 2H), 2.69-2.64 (m, 4H). LC-MS (M+H): 302.29.

### Experimental Procedures for Compounds 11, 15, and 16:

### Synthetic Schemes:

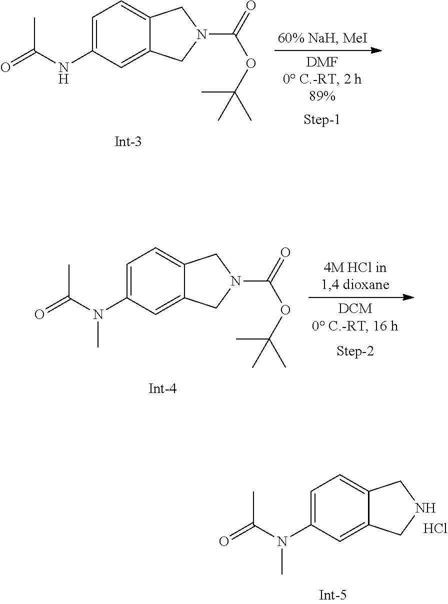 [SMILES: CC(=O)Nc1ccc2c(c1)CN(C(=O)OC(C)(C)C)C2 ; CC(=O)N(C)c1ccc2c(c1)CN(C(=O)OC(C)(C)C)C2 ; CC(=O)N(C)c1ccc2c(c1)CNC2 ; Cl]

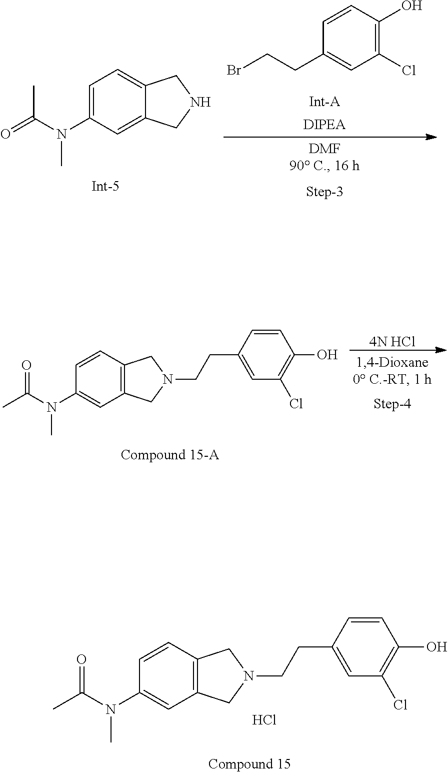

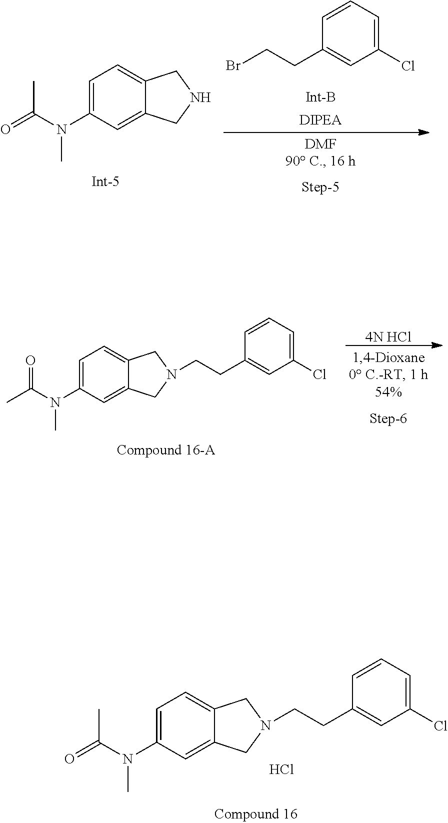

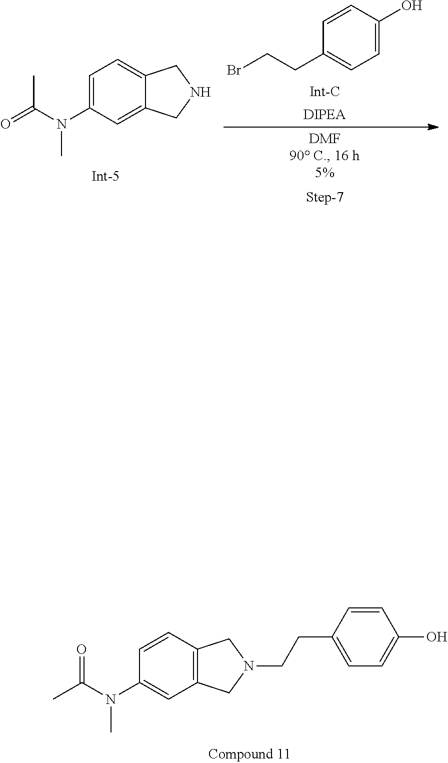 [SMILES: CC(=O)N(C)c1ccc2c(c1)CNC2 ; Oc1ccc(CCBr)cc1 ; CC(=O)N(C)c1ccc2c(c1)CN(CCc1ccc(O)cc1)C2]

### Synthesis of Compound Int-4:

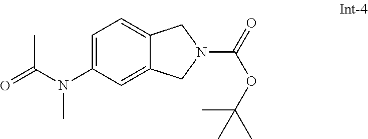 [SMILES: CC(=O)N(C)c1ccc2c(c1)CN(C(=O)OC(C)(C)C)C2]

tert-Butyl 5-(N-methylacetamido) isoindoline-2-carboxylate: To a stirred mixture of Int-3 (1.6 g, 5.79 mmol, 1.0 eq.) in N,N-dimethylformamide (20 mL) was added 60% sodium hydride (0.278 g, 6.95 mmol, 1.2 eq.) at 0° C. and the reaction mixture was stirred at the same temperature for 15 minutes, followed by the addition of methyl iodide (0.541 mL, 8.68 mmol, 1.5 eq.) at room temperature and the reaction mixture was stirred at the same temperature for 2 h. The progress of the reaction was monitored by TLC and LCMS. After completion of the reaction, the reaction mixture was diluted with ice-water and extracted with ethyl acetate twice. The combined organic layer was washed with water, brine, dried over by anhydrous Na₂SO₄, filtered and concentrated under reduced pressure to give Int-4 (1.5 g, Y: 89%) as brown gum. ¹H NMR (400 MHz, CHLOROFORM-d) δ=7.34-7.30 (m, 1H), 7.14-7.02 (m, 2H), 4.69 (d, J=14.2 Hz, 4H), 3.25 (s, 3H), 1.87 (s, 3H), 1.52 (s, 9H). LC-MS (M+H): 291.24.

### Synthesis of Compound Int-5:

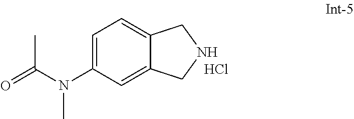

N-(Isoindolin-5-yl)-N-methylacetamide: To a stirred solution of Int-4 (1.5 g, 5.17 mmol, 1.0 eq.) in dichloromethane (20 mL) was added 4N HCl in 1,4-dioxane (7 mL, 25.83 mmol, 5.0 eq.) at 0° C. and the reaction mixture was stirred at the room temperature for 16 h. The progress of the reaction was monitored by TLC and LCMS. After completion of the reaction, the reaction mixture was concentrated under reduced pressure to give Int-5 (1.0 g, crude) as green gum. ¹H NMR (400 MHz, DMSO-d₆) δ=9.99 (br. s, 1H), 7.46 (d, J=7.8 Hz, 1H), 7.38 (br. s, 1H), 7.32 (d, J=7.6 Hz, 1H), 4.51 (t, J=5.6 Hz, 4H), 4.41 (s, 6H).

### Synthesis of Compound 15-A:

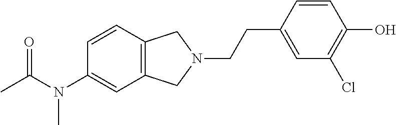 [SMILES: CC(=O)N(C)c1ccc2c(c1)CN(CCc1ccc(O)c(Cl)c1)C2]

N-(2-(3-Chloro-4-hydroxyphenethyl) isoindolin-5-yl)-N-methylacetamide: To a stirred solution of Int-5 (0.150 g, 0.66 mmol, 1.0 eq.) in N,N-dimethylformamide (3 mL) were added N,N-diisopropylethylamine (0.34 mL, 1.98 mmol, 3.0 eq.) and 4-(2-bromoethyl)-2-chlorophenol (0.187 g, 0.79 mmol, 1.2 eq.) at room temperature and the reaction mixture was stirred at 90° C. for 16 h. The progress of the reaction was monitored by TLC and LCMS. After completion of the reaction, the reaction mixture was poured into ice-water and extracted with ethyl acetate twice. The combined organic layer was washed with water, brine, dried over by anhydrous Na₂SO₄, filtered and concentrated under reduced pressure to give crude. The crude was purified by prep. HPLC to afford compound 15-A (20 mg, Impure) as brown gum. LC-MS (M+H): 345.13.

### Synthesis of Compound 15:

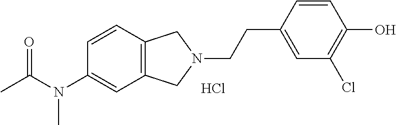 [SMILES: CC(=O)N(C)c1ccc2c(c1)CN(CCc1ccc(O)c(Cl)c1)C2 ; Cl]

N-(2-(3-Chloro-4-hydroxyphenethyl) isoindolin-5-yl)-N-methylacetamide hydrochloride: To a stirred solution of compound 15-A (20 mg, 0.06 mmol, 1.0 eq.) in dichloromethane (2 mL) was added 4N HCl in 1,4-dioxane (0.01 mL) at 0° C. and the reaction mixture was stirred at the room temperature for 1 h. The progress of the reaction was monitored by TLC and LCMS. After completion of the reaction, the reaction mixture was concentrated under reduced pressure to give compound 15 (20 mg, quantitative yield) as a pale-green solid. ¹H NMR (400 MHz, DMSO-d₆) δ=11.30 (br. s, 1H), 10.14 (s, 1H), 7.52-7.30 (m, 4H), 7.08 (dd, J=2.0, 8.3 Hz, 1H), 6.95 (d, J=8.3 Hz, 1H), 4.86-4.75 (m, 2H), 4.65-4.49 (m, 2H), 3.59 (d, J=4.5 Hz, 2H), 3.16 (d, J=6.5 Hz, 3H), 2.98-2.94 (m, 2H), 1.78 (br. s, 3H). LC-MS (M+H): 345.21.

### Synthesis of Compound 16-A:

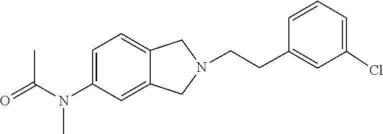 [SMILES: CC(=O)N(C)c1ccc2c(c1)CN(CCc1cccc(Cl)c1)C2]

N-(2-(3-Chlorophenethyl) isoindolin-5-yl)-N-methylacetamide: To a stirred solution of Int-5 (0.200 g, 0.88 mmol, 1.0 eq.) in N,N-dimethylformamide (5 mL) were added N,N-diisopropylethylamine (0.46 mL, 2.65 mmol, 3.0 eq.) and 1-(2-bromoethyl)-3-chlorobenzene (0.232 g, 1.06 mmol, 1.2 eq.) at room temperature and the reaction mixture was stirred at 90° C. for 16 h. The progress of the reaction was monitored by TLC and LCMS. After completion of the reaction, the reaction mixture was poured into ice-water and extracted with ethyl acetate twice. The combined organic layer was washed with water, brine, dried over by anhydrous Na₂SO₄, filtered and concentrated under reduced pressure to give crude. The crude was purified by column chromatography using silica gel (230-400 mesh), eluted with 4-5% methanol/dichloromethane to afford compound 16-A (130 mg, Impure) as brown gum. ¹H NMR (400 MHz, CDCl₃) δ=7.25-7.19 (m, 4H), 7.15-7.14 (m, 1H), 7.03-7.00 (m, 2H), 3.99 (s, 4H), 3.24 (s, 3H), 3.02-2.98 (m, 2H), 2.90-2.86 (m, 2H), 1.86 (s, 3H). LC-MS (M+H): 329.26.

### Synthesis of Compound 16:

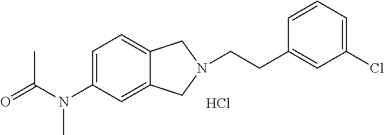 [SMILES: CC(=O)N(C)c1ccc2c(c1)CN(CCc1cccc(Cl)c1)C2 ; Cl]

N-(2-(3-Chlorophenethyl) isoindolin-5-yl)-N-methylacetamide hydrochloride: To a stirred solution of compound 16-A (130 mg, 0.39 mmol, 1.0 eq.) in dichloromethane (5 mL) was added 4N HCl in 1,4-dioxane (0.2 mL) at 0° C. and the reaction mixture was stirred at the room temperature for 1 h. The progress of the reaction was monitored by TLC and LCMS. After completion of the reaction, the reaction mixture was concentrated under reduced pressure to give compound 16 (70 mg, Y: 54%) as a green solid. ¹H NMR (400 MHz, DMSO-d₆) δ=11.47 (br. s, 1H), 7.58-7.18 (m, 7H), 4.90-4.49 (m, 4H), 3.69-3.56 (m, 2H), 3.25-2.93 (m, 5H), 1.79 (s, 3H). LC-MS (M+H): 329.25.

### Synthesis of Compound 11:

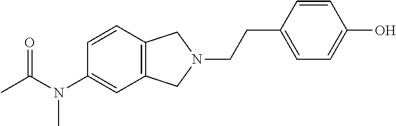 [SMILES: CC(=O)N(C)c1ccc2c(c1)CN(CCc1ccc(O)cc1)C2]

N-(2-(4-Hydroxyphenethyl) isoindolin-5-yl)-N-methylacetamide: To a stirred solution of Int-5 (0.130 g, 0.68 mmol, 1.0 eq.) in N,N-dimethylformamide (3 mL) were added N,N-diisopropylethylamine (0.357 mL, 2.05 mmol, 3.0 eq.) and 4-(2-bromoethyl) phenol (0.164 g, 0.82 mmol, 1.2 eq.) at room temperature and the reaction mixture was stirred at 90° C. for 16 h. The progress of the reaction was monitored by TLC and LCMS. After completion of the reaction, the reaction mixture was poured into ice-water and extracted with ethyl acetate twice. The combined organic layer was washed with water, brine, dried over by anhydrous Na₂SO₄, filtered and concentrated under reduced pressure to give crude. The crude was purified by prep. HPLC to afford compound 11 (10 mg, Y: 5%) as an off-white solid. ¹H NMR (400 MHz, DMSO-d₆) δ=9.13 (s, 1H), 7.31-7.11 (m, 3H), 7.07-7.03 (m, 2H), 6.70-6.64 (m, 2H), 3.92-3.88 (m, 4H), 3.11 (s, 3H), 2.89-2.78 (m, 2H), 2.72-2.66 (m, 2H), 1.74 (s, 3H). LC-MS (M+H): 311.21.

### Experimental Procedures for Compound 12:

### Synthetic Scheme:

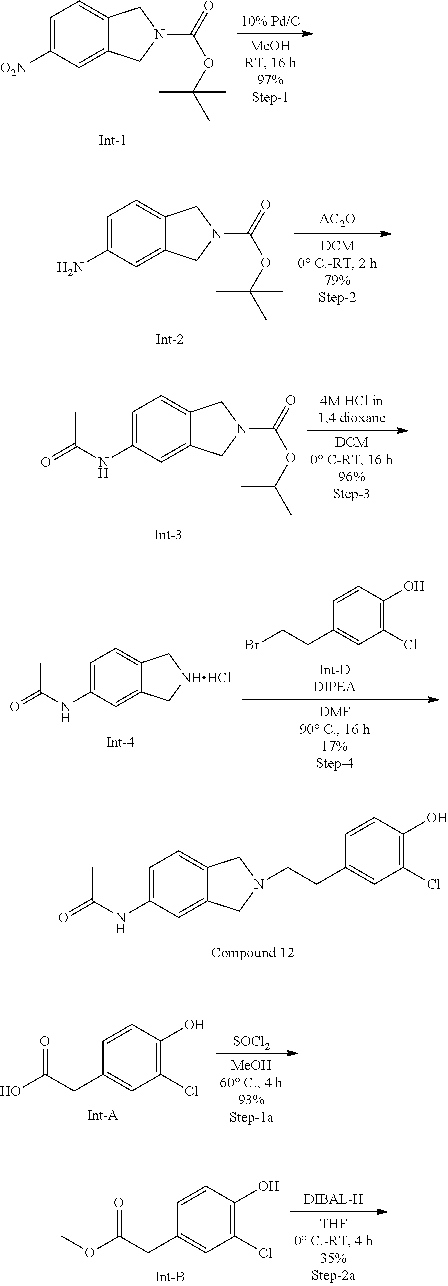

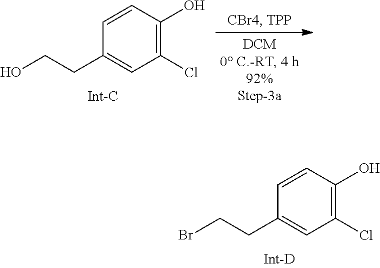 [SMILES: OCCc1ccc(O)c(Cl)c1 ; Oc1ccc(CCBr)cc1Cl]

### Synthesis of Compound Int-B:

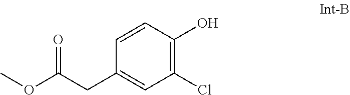

Methyl 2-(3-chloro-4-hydroxyphenyl) acetate: To a stirred mixture of 2-(3-chloro-4-hydroxyphenyl) acetic acid (1.0 g, 5.36 mmol, 1.0 eq.) in methanol (15 mL) was added thionyl chloride (3.88 mL, 53.59 mmol, 10.0 eq.) at 0° C. and the reaction mixture was stirred at 60° C. for 4 h. The progress of the reaction was monitored by TLC and LCMS. After completion of the reaction, the reaction mixture was concentrated under reduced pressure to give crude. The crude was co-distilled with dichloromethane twice to give Int-B (1.0 g, Y: 93%) as brown oil. ¹H NMR (400 MHz, CDCl₃) δ 7.25-7.24 (m, 1H), 7.09 (dd, J=2.0, 8.0 Hz, 1H), 6.98-6.95 (m, 1H), 3.70 (s, 3H), 3.57 (s, 2H).

### Synthesis of Compound Int-C:

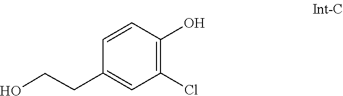 [SMILES: OCCc1ccc(O)c(Cl)c1]

2-Chloro-4-(2-hydroxyethyl) phenol: To a stirred mixture of Int-B (0.5 g, 2.49 mmol, 1.0 eq.) in tetrahydrofuran (5 mL) was added DIBAL-H (1 M in toluene) (13.4 mL, 13.46 mmol, 3 equiv.) at 0° C. and the reaction mixture was stirred at room temperature for 4 h. The progress of the reaction was monitored by TLC and LCMS. After completion of the reaction, the reaction mixture was quenched with saturated ammonium chloride solution and extracted with ethyl acetate twice. The combined organic layer was dried over anhydrous Na₂SO₄, filtered and concentrated under reduced pressure to give crude. The crude was purified by column chromatography using silica gel (100-200 mesh), eluted with 30-50% ethyl acetate/pet ether to afford Int-C (0.150 g, Y: 35%) as colourless gum. ¹H NMR (400 MHz, CHLOROFORM-d) δ=7.20 (d, J=2.0 Hz, 1H), 7.07-7.02 (m, 1H), 6.99-6.93 (m, 1H), 5.48 (br. s, 1H), 3.83 (t, J=6.5 Hz, 2H), 2.79 (t, J=6.4 Hz, 2H). One exchangeable proton is not observed in ¹H NMR. No ionization in LC-MS.

### Synthesis of Compound Int-D:

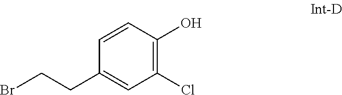 [SMILES: Oc1ccc(CCBr)cc1Cl]

4-(2-Bromoethyl)-2-chlorophenol: To a stirred solution of Int-C (0.8 g, 4.63 mmol, 1.0 eq.) in dichloromethane (50 mL) were added carbon tetrabromide (2.30 g, 6.95 mmol, 1.5 eq.) and triphenylphosphine (1.82 g, 6.95 mmol, 1.5 eq.) at 0° C. and the reaction mixture was stirred at room temperature for 4 h. The progress of the reaction was monitored by TLC and LCMS. After completion of the reaction, the reaction mixture was poured into ice-water and extracted with dichloromethane twice. The combined organic layer was washed with water, brine, dried over by anhydrous Na₂SO₄, filtered and concentrated under reduced pressure to give crude. The crude was purified by column chromatography using silica gel (100-200 mesh), eluted with 10-50% ethyl acetate/pet ether to afford Int-D (1.0 g, Y: 92%) as colourless oil. ¹H NMR (400 MHz, DMSO-d₆) δ=9.98 (s, 1H), 7.25 (d, J=2.0 Hz, 1H), 7.03 (dd, J=2.4, 8.4 Hz, 1H), 6.89 (d, J=8.8 Hz, 1H), 3.72-3.62 (m, 2H), 3.05-2.99 (m, 2H).

### Synthesis of Compound Int-2:

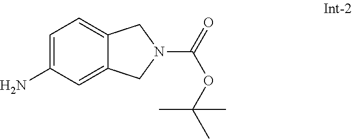 [SMILES: CC(C)(C)OC(=O)N1Cc2ccc(N)cc2C1]

tert-Butyl 5-aminoisoindoline-2-carboxylate: To a stirred mixture of tert-butyl 5-nitroisoindoline-2-carboxylate (3.0 g, 11.35 mmol, 1.0 eq.) in methanol (30 mL) was added 10% Pd/C (0.6 g) at room temperature and the reaction mixture was hydrogenated under balloon pressure for 16 h. The progress of the reaction was monitored by TLC and LCMS. After completion of the reaction, the reaction mixture was filtered through Celite bed and washed with methanol. The filtrate was concentrated under reduced pressure to give Int-2 (2.6 g, Y: 97%) as brown gum. LC-MS (M+H): 235.07.

### Synthesis of Compound Int-3:

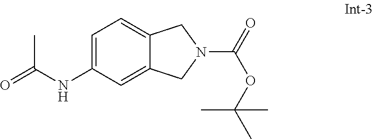 [SMILES: CC(=O)Nc1ccc2c(c1)CN(C(=O)OC(C)(C)C)C2]

tert-Butyl 5-acetamidoisoindoline-2-carboxylate: To a stirred mixture of Int-2 (1.5 g, 6.40 mmol, 1.0 eq.) in dichloromethane (20 mL) was added acetic anhydride (3.20 mL, 32.01 mmol, 5.0 eq.) at 0° C. and the reaction mixture was stirred at room temperature for 2 h. The progress of the reaction was monitored by TLC and LCMS. After completion of the reaction, the reaction mixture was diluted with ice-water and extracted with dichloromethane twice. The combined organic layer was washed with saturated sodium bicarbonate solution, brine, dried over anhydrous Na₂SO₄, filtered and concentrated under reduced pressure to give Int-3 (1.4 g, Y: 79%) as an off-white solid. ¹H NMR (500 MHz, CHLOROFORM-d) δ=7.60-7.52 (m, 1H), 7.30 (d, J=8.0 Hz, 1H), 7.20-7.14 (m, 2H), 4.66-4.60 (m, 4H), 2.18 (s, 3H), 1.52 (s, 9H). LC-MS (M+H): 277.03.

### Synthesis of Compound Int-4:

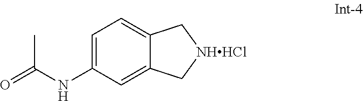

N-(Isoindolin-5-yl) acetamide hydrochloride: To a stirred mixture of Int-3 (0.7 g, 2.53 mmol, 1.0 eq.) in dichloromethane (10 mL) was added 4N HCl in 1,4-dioxane (3.17 mL, 12.67 mmol, 5.0 eq.) at 0° C. and the reaction mixture was stirred at room temperature for 16 h. The progress of the reaction was monitored by TLC and LCMS. After completion of the reaction, the reaction mixture was concentrated under reduced pressure to give Int-5 (0.520 g, Y: 96%) as an off-white solid. ¹H NMR (400 MHz, DMSO-d₆) δ=10.20 (s, 1H), 9.95 (br. s, 2H), 7.75 (s, 1H), 7.46 (dd, J=1.3, 8.3 Hz, 1H), 7.30 (d, J=8.3 Hz, 1H), 4.47-4.40 (m, 4H), 2.05 (s, 3H). LC-MS (M+H): 177.06.

### Synthesis of Compound 12:

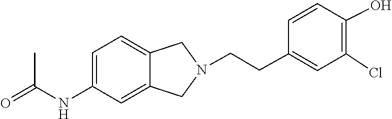 [SMILES: CC(=O)Nc1ccc2c(c1)CN(CCc1ccc(O)c(Cl)c1)C2]

N-(2-(3-Chloro-4-hydroxyphenethyl) isoindolin-5-yl) acetamide: To a stirred mixture of Int-4 (0.130 g, 0.61 mmol, 1.0 eq.) in N,N-Dimethylformamide (3 mL) were added N, N-diisopropylethylamine (0.32 mL, 1.83 mmol, 3.0 eq.) and 4-(2-bromoethyl)-2-chlorophenol (0.172 g, 0.73 mmol, 1.2 eq.) at room temperature and the reaction mixture was stirred at 90° C. for 16 h. The progress of the reaction was monitored by TLC and LCMS. After completion of the reaction, the reaction mixture was diluted with ice-water and extracted with ethyl acetate twice. The combined organic layer was washed with brine, dried over anhydrous Na₂SO₄, filtered and concentrated under reduced pressure to give crude. The crude was purified by prep. HPLC to afford compound 12 (35 mg, Y: 17%) as an off-white solid. ¹H NMR (400 MHz, DMSO-d₆) δ=9.86 (s, 2H), 7.52 (s, 1H), 7.30 (dd, J=1.6, 8.1 Hz, 1H), 7.23 (d, J=1.8 Hz, 1H), 7.12 (d, J=8.0 Hz, 1H), 7.03 (dd, J=2.0, 8.3 Hz, 1H), 6.87 (d, J=8.3 Hz, 1H), 3.83-3.80 (m, 4H), 2.87-2.77 (m, 2H), 2.73-2.65 (m, 2H), 2.02 (s, 3H). LC-MS (M+H): 331.20.

### Experimental Procedures for Compound 13:

### Synthetic Scheme:

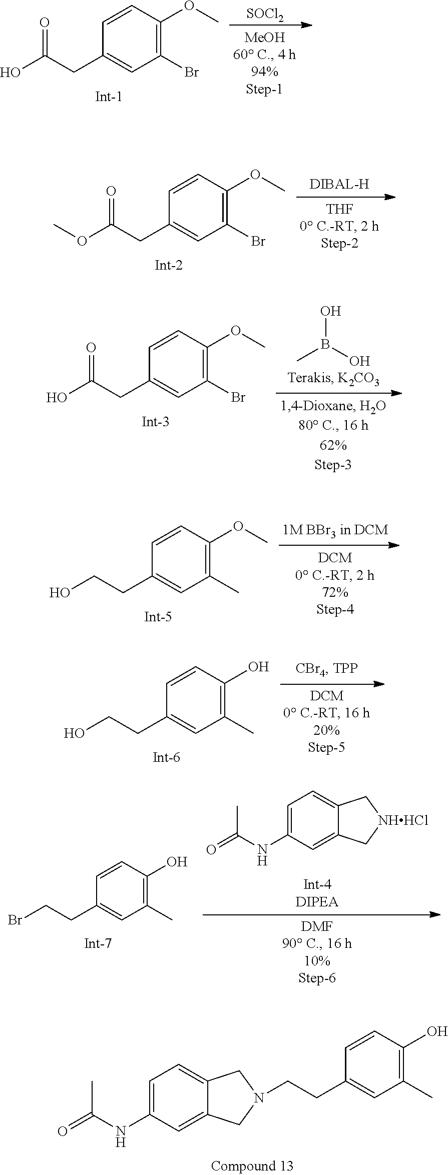

### Synthesis of Compound Int-2:

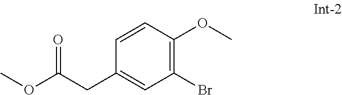

Methyl 2-(3-bromo-4-methoxyphenyl) acetate: To a stirred mixture of 2-(3-bromo-4-methoxyphenyl) acetic acid (3.0 g, 12.24 mmol, 1.0 eq.) in methanol (30 mL) was added thionyl chloride (8.88 mL, 122.41 mmol, 10.0 eq.) at 0° C. and the reaction mixture was stirred at 60° C. for 4 h. The progress of the reaction was monitored by TLC and LCMS. After completion of the reaction, the reaction mixture was concentrated under reduced pressure to 10 give crude. The crude was co-distilled with dichloromethane twice to give Int-2 (3.0 g, Y: 94%) as brown oil. ¹H NMR (400 MHz, CHLOROFORM-d) δ=7.47 (d, J=2.0 Hz, 1H), 7.19 (dd, J=2.1, 8.4 Hz, 1H), 6.85 (d, J=8.5 Hz, 1H), 3.88 (s, 3H), 3.70 (s, 3H), 3.54 (s, 2H).

### Synthesis of Compound Int-3:

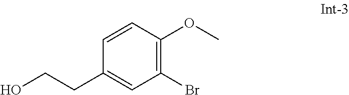

2-(3-Bromo-4-methoxyphenyl) ethan-1-ol: To a stirred mixture of Int-2 (3.0 g, 11.58 mmol, 1.0 eq.) in tetrahydrofuran (60 mL) was added DIBAL-H (1 M in toluene) (34.74 mL, 34.74 mmol, 3 eq.) at 0° C. and the reaction mixture was stirred at room temperature for 2 h. The progress of the reaction was monitored by TLC and LCMS. After completion of the reaction, the reaction mixture was quenched with saturated ammonium chloride solution and extracted with ethyl acetate twice. The combined organic layer was dried over anhydrous Na₂SO₄, filtered and concentrated under reduced pressure to give Int-3 (3.0 g, crude) as pale-yellow oil. ¹H NMR (500 MHz, CHLOROFORM-d) 8=7.42 (d, J=2.1 Hz, 1H), 7.13 (dd, J=2.1, 8.2 Hz, 1H), 6.85 (d, J=8.5 Hz, 1H), 3.88 (s, 3H), 3.83 (t, J=6.4 Hz, 2H), 2.79 (t, J=6.6 Hz, 2H). One exchangeable proton is not observed in ¹H NMR.

### Synthesis of Compound Int-5:

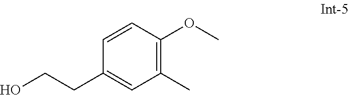

2-(4-Methoxy-3-methylphenyl) ethan-1-ol: The stirred mixture of Int-3 (1.0 g, 4.33 mmol, 1.0 eq.), methylboronic acid (0.389 g, 6.49 mmol, 1.5 eq.) and potassium carbonate (1.196 g, 8.65 mmol, 2.0 eq.) in 1,4-dioxane (10 mL): water (1 mL) was degassed with argon for 5 minutes, followed by the addition of Tetrakis (triphenylphosphine) palladium (0) (0.5 g, 0.43 mmol, 0.1 eq.) at room temperature and the reaction mixture was stirred at 80° C. for 16 h. The progress of the reaction was monitored by TLC and LCMS. After completion of the reaction, the reaction mixture was filtered through Celite bed and washed with dichloromethane twice. The filtrate was concentrated under reduced pressure to give crude. The crude was purified by column chromatography using silica gel (100-200 mesh), eluted with 10-20% ethyl acetate/pet ether to afford Int-5 (0.450 g, Y: 62%) as pale-yellow oil. ¹H NMR (400 MHz, CHLOROFORM-d) δ=7.05-6.97 (m, 2H), 6.77 (d, J=8.0 Hz, 1H), 3.85-3.77 (m, 5H), 2.78 (t, J=6.5 Hz, 2H), 2.21 (s, 3H), 1.41 (t, J=5.8 Hz, 1H).

### Synthesis of Compound Int-6:

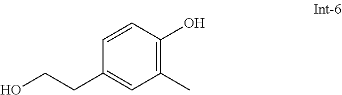

4-(2-Hydroxyethyl)-2-methylphenol: To a stirred solution of Int-3 (0.640 g, 3.85 mmol, 1.0 eq.) in dichloromethane (15 mL) was added boron tribromide (38 mL, 38.50 mmol, 10.0 eq., 1.0M in dichloromethane) at 0° C. and the reaction mixture was stirred at the room temperature for 2 h. The progress of the reaction was monitored by TLC and LCMS. After completion of the reaction, the reaction mixture was concentrated under reduced pressure to give residue. The residue was diluted with ice-water, and basified by 25% aqueous ammonia solution and extracted with ethyl acetate twice. The crude was purified by column chromatography using silica gel (100-200 mesh), eluted with 10-50% ethyl acetate/pet ether to afford Int-6 (0.420 g, Y: 72%) as pale-yellow oil. ¹H NMR (400 MHz, CHLOROFORM-d) δ=6.99-6.87 (m, 2H), 6.72 (d, J=8.1 Hz, 1H), 4.70 (s, 1H), 3.52 (t, J=7.7 Hz, 2H), 3.06 (t, J=7.7 Hz, 2H), 2.24 (s, 3H). One exchangeable proton is not observed in ¹H NMR. No ionization in LC-MS.

### Synthesis of Compound Int-7:

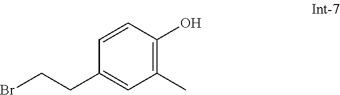

4-(2-Bromoethyl)-2-methylphenol: To a stirred solution of Int-6 (0.420 g, 2.76 mmol, 1.0 eq.) in dichloromethane (10 mL) were added triphenyl phosphine (1.08 g, 4.14 mmol, 1.5 eq.) and carbon tetrabromide (1.098 g, 3.31 mmol, 1.2 eq.) at 0° C. and the reaction mixture was stirred at the room temperature for 16 h. The progress of the reaction was monitored by TLC and LCMS. After completion of the reaction, the reaction mixture was concentrated under reduced pressure to give crude. The crude was purified by column chromatography using silica gel (100-200 mesh), eluted with 15-30% ethyl acetate/pet ether to afford Int-7 (0.120 g, Y: 20%) as a colourless oil. ¹H NMR (500 MHz, DMSO-d₆) δ=9.12 (s, 1H), 6.94 (d, J=1.5 Hz, 1H), 6.87 (dd, J=2.0, 8.1 Hz, 1H), 6.68 (d, J=7.9 Hz, 1H), 3.62 (t, J=7.5 Hz, 2H), 2.96 (t, J=7.5 Hz, 2H), 2.09 (s, 3H).

### Synthesis of Compound 13:

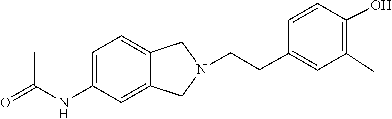 [SMILES: CC(=O)Nc1ccc2c(c1)CN(CCc1ccc(O)c(C)c1)C2]

N-(2-(4-Hydroxy-3-methylphenethyl) isoindolin-5-yl) acetamide: To a stirred solution of Int-4 (0.100 g, 0.47 mmol, 1.0 eq.) in N,N-Dimethylformamide (3 mL) were added N, N-diisopropylethylamine (0.24 mL, 1.41 mmol, 3.0 eq.) and Int-7 (0.101 g, 0.47 mmol, 1.0 eq.) at room temperature and the reaction mixture was stirred at 90° C. for 16 h. The progress of the reaction was monitored by TLC and LCMS. After completion of the reaction, the reaction mixture was diluted with ice-water and extracted with ethyl acetate twice. The combined organic layer was washed with brine, dried over anhydrous Na₂SO₄, filtered and concentrated under reduced pressure to give crude. The crude was purified by column chromatography using silica gel (230-400 mesh), eluted with 5-10% methanol/dichloromethane to afford compound 13 (15 mg, Y: 10%) as pale-brown solid. ¹H NMR (400 MHz, DMSO-d₆) δ=9.86 (s, 1H), 8.99 (s, 1H), 7.52 (s, 1H), 7.32-7.22 (m, 1H), 7.12 (d, J=8.3 Hz, 1H), 6.93 (s, 1H), 6.86 (dd, J=1.8, 8.0 Hz, 1H), 6.66 (d, J=8.0 Hz, 1H), 3.83 (d, J=12.0 Hz, 4H), 2.85-2.77 (m, 2H), 2.69-2.60 (m, 2H), 2.08 (s, 3H), 2.02 (s, 3H). LC-MS (M+H): 311.29.

### Experimental Procedures for Compound 14:

### Synthetic Scheme:

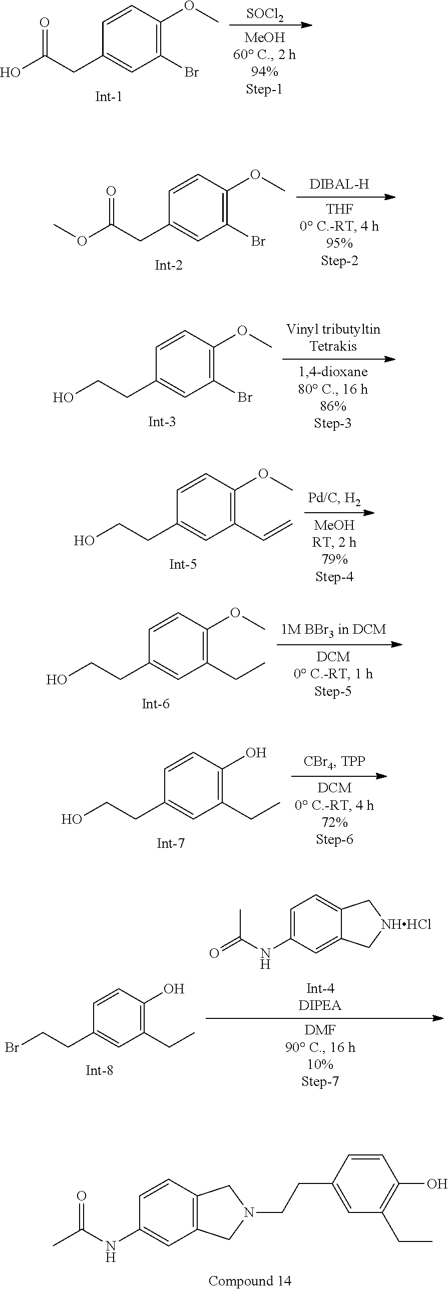

### Synthesis of Compound Int-2:

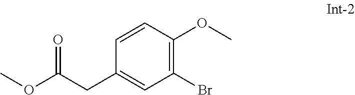

Methyl 2-(3-bromo-4-methoxyphenyl) acetate: To a stirred mixture of 2-(3-bromo-4-methoxyphenyl) acetic acid (2.0 g, 8.16 mmol, 1.0 eq.) in methanol (15 mL) was added thionyl chloride (5.92 mL, 81.61 mmol, 10.0 eq.) at 0° C. and the reaction mixture was stirred at 60° C. for 2 h. The progress of the reaction was monitored by TLC and LCMS. After completion of the reaction, the reaction mixture was concentrated under reduced pressure to give crude. The crude was co-distilled with dichloromethane twice to afford Int-2 (2.0 g, Y: 94%) as brown oil. ¹H NMR (400 MHz, CHLOROFORM-d) δ=7.47 (d, J=2.2 Hz, 1H), 7.18 (dd, J=2.1, 8.4 Hz, 1H), 6.85 (d, J=8.3 Hz, 1H), 3.88 (s, 3H), 3.69 (s, 3H), 3.54 (s, 2H). LC-MS (M+H): 259.05.

### Synthesis of Compound Int-3:

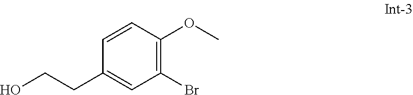

2-(3-Bromo-4-methoxyphenyl) ethan-1-ol: To a stirred mixture of Int-2 (2.0 g, 7.72 mmol, 1.0 eq.) in tetrahydrofuran (50 mL) was added DIBAL-H (1 M in toluene) (23.16 mL, 23.16 mmol, 3.0 eq.) at 0° C. and the reaction mixture was stirred at the room temperature for 4 h. The progress of the reaction was monitored by TLC and LCMS. After completion of the reaction, the reaction mixture was quenched with ammonium chloride solution and extracted with ethyl acetate twice. The combined organic layer was washed with water, brine, dried over anhydrous Na₂SO₄, filtered and concentrated under reduced pressure to give Int-3 (1.7 g, Y: 95%) as brown oil. ¹H NMR (400 MHz, CHLOROFORM-d) δ=7.42 (d, J=2.0 Hz, 1H), 7.13 (dd, J=2.1, 8.4 Hz, 1H), 6.85 (d, J=8.3 Hz, 1H), 3.88 (s, 3H), 3.83 (t, J=6.6 Hz, 2H), 2.79 (t, J=6.5 Hz, 2H). One exchangeable proton is not observed in ¹H NMR. LC-MS (M−H₂O+H): 213.04.

### Synthesis of Compound Int-5:

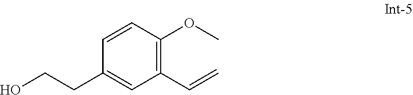

2-(4-Methoxy-3-vinylphenyl) ethan-1-ol: The mixture of Int-3 (1.5 g, 6.49 mmol, 1.0 eq.) and Tributylvinyltin (3.08 g, 9.74 mmol, 1.5 eq.) in 1,4-dioxane (10 mL) was degassed with argon for 5 minutes, followed by the addition of Tetrakis (triphenylphosphine) palladium (0) (0.750 g, 0.65 mmol, 0.1 eq.) at room temperature and the reaction mixture was stirred at 80° C. for 16 h. The progress of the reaction was monitored by TLC and LCMS. After completion of the reaction, the reaction mixture was filtered through Celite bed and washed with dichloromethane. The filtrate was concentrated under reduced pressure to give crude. The crude was purified by column chromatography using silica gel (100-200 mesh), eluted with 10-50% ethyl acetate/pet ether to afford Int-5 (1.0 g, Y: 86%) as a pale-yellow oil. ¹H NMR (500 MHz, CHLOROFORM-d) δ=7.32 (d, J=2.1 Hz, 1H), 7.10 (dd, J=2.3, 8.4 Hz, 1H), 7.02 (dd, J=11.1, 17.9 Hz, 1H), 6.83 (d, J=8.5 Hz, 1H), 5.74 (dd, J=1.4, 17.9 Hz, 1H), 5.26 (dd, J=1.4, 11.1 Hz, 1H), 3.89-3.79 (m, 6H), 2.82 (t, J=6.6 Hz, 2H). LC-MS (M+H): 179.10.

### Synthesis of Compound Int-6:

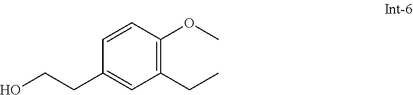

2-(3-Ethyl-4-methoxyphenyl) ethan-1-ol: To a stirred mixture of Int-5 (1.0 g, 5.61 mmol, 1.0 eq.) in methanol (50 mL) was added 10% Pd/C (0.5 g) at room temperature and the reaction mixture was hydrogenated under balloon for 2 h. The progress of the reaction was monitored by TLC and LCMS. After completion of the reaction, the reaction mixture was filtered through Celite bed and washed with methanol. The filtrate was concentrated under reduced pressure to give Int-6 (0.8 g, Y: 79%) as pale-yellow oil. ¹H NMR (500 MHz, CHLOROFORM-d) δ=7.04-6.99 (m, 2H), 6.79 (d, J=7.9 Hz, 1H), 3.85-3.78 (m, 6H), 2.80 (t, J=6.6 Hz, 2H), 2.62 (q, J=7.6 Hz, 2H), 1.19 (t, J=7.5 Hz, 3H). LC-MS (M−H₂O+H): 163.98.

### Synthesis of Compound Int-7:

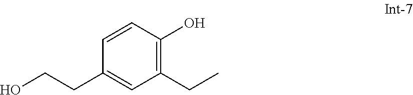

2-Ethyl-4-(2-hydroxyethyl) phenol: To a stirred mixture of Int-6 (1.0 g, 5.55 mmol, 1.0 eq.) in dichloromethane (50 mL) was added 1M BBr₃ in DCM (27.74 mL, 27.74 mmol, 5.0 eq.) at 0° C. and the reaction mixture was stirred at room temperature for 1 h. The progress of the reaction was monitored by TLC and LCMS. After completion of the reaction, the reaction mixture was concentrated under reduced pressure to give residue. The residue was basified with aqueous ammonia solution and extracted with ethyl acetate twice. The combined organic layer was washed with water, brine, dried over anhydrous Na₂SO₄, filtered and concentrated under reduced pressure to give Int-7 (0.8 g, Impure) as brown oil. ¹H NMR (400 MHz, CHLOROFORM-d) δ=6.99-6.96 (m, 1H), 6.91 (dd, J=2.2, 8.1 Hz, 1H), 6.70 (d, J=8.1 Hz, 1H), 3.52 (t, J=7.8 Hz, 2H), 3.07 (t, J=7.7 Hz, 2H), 2.62 (q, J=7.6 Hz, 2H), 1.23 (t, J=7.6 Hz, 3H). Two exchangeable protons are not observed in ¹H NMR LC-MS (M−H₂O+H): 149.02.

### Synthesis of Compound Int-8:

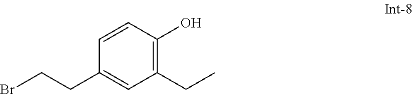

4-(2-Bromoethyl)-2-ethylphenol: To a stirred mixture of Int-7 (1.0 g, 6.02 mmol, 1.0 eq.) in dichloromethane (20 mL) were added triphenylphosphine (2.367 g, 9.02 mmol, 1.5 eq.) and carbon tetrabromide (2.39 g, 7.22 mmol, 1.2 eq.) at 0° C. and the reaction mixture was stirred at the room temperature for 4 h. The progress of the reaction was monitored by TLC and LCMS. After completion of the reaction, the reaction mixture was concentrated under reduced pressure to give crude. The crude was purified by column chromatography using silica gel (100-200 mesh), eluted with 15-30% ethyl acetate/pet ether to afford Int-8 (1.0 g, Y: 72%) as colourless oil. ¹H NMR (400 MHz, DMSO-d₆) δ=9.09 (s, 1H), 6.97-6.93 (m, 1H), 6.87 (dd, J=2.3, 8.2 Hz, 1H), 6.69 (d, J=8.1 Hz, 1H), 3.62 (t, J=7.6 Hz, 2H), 2.97 (t, J=7.5 Hz, 2H), 2.51-2.49 (m, 2H), 1.11 (t, J=7.6 Hz, 3H).

### Synthesis of Compound 14:

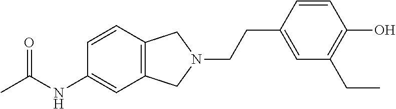 [SMILES: CCc1cc(CCN2Cc3ccc(NC(C)=O)cc3C2)ccc1O]

N-(2-(3-Ethyl-4-hydroxyphenethyl) isoindolin-5-yl) acetamide: To a stirred solution of N-(isoindolin-5-yl) acetamide hydrochloride (130 mg, 0.61 mmol, 1.0 eq.) in N,N-Dimethylformamide (3 mL) were added N, N-diisopropylethylamine (0.32 mL, 1.83 mmol, 3.0 eq.) and Int-8 (168 mg, 0.73 mmol, 1.2 eq.) at room temperature and the reaction mixture was stirred at 90° C. for 16 h. The progress of the reaction was monitored by TLC and LCMS. After completion of the reaction, the reaction mixture was diluted with ice-water and extracted with ethyl acetate twice. The combined organic layer was washed with water, brine, dried over anhydrous Na₂SO₄, filtered and concentrated under reduced pressure to give crude. The crude was purified by column chromatography using silica gel (100-200 mesh), eluted with 15-30% ethyl acetate/pet ether, followed by pep. HPLC to afford compound 14 (20 mg, Y: 10%) as an off-white solid. ¹H NMR (401 MHz, DMSO-d₆) δ=9.86 (s, 1H), 8.97 (s, 1H), 7.51 (s, 1H), 7.30 (dd, J=1.5, 8.0 Hz, 1H), 7.12 (d, J=8.3 Hz, 1H), 6.93 (d, J=2.0 Hz, 1H), 6.86 (dd, J=2.1, 8.1 Hz, 1H), 6.67 (d, J=8.3 Hz, 1H), 3.84 (s, 2H), 3.81 (s, 2H), 2.85-2.77 (m, 2H), 2.70-2.61 (m, 2H), 2.54-2.52 (m, 2H), 2.02 (s, 3H), 1.11 (t, J=7.5 Hz, 3H). LC-MS (M+H): 325.27.

What is claimed is:

1. A compound, or a pharmaceutically acceptable salt thereof, having the formula: 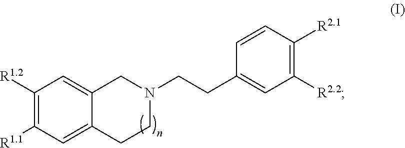
wherein
n is 0 or 1;
R^{1.1} and R^{1.2} are independently hydrogen, halogen, —C(O)NR^{1A}R^{1B}, or —NR^{1A}C(O)R^{1C};
R^{2.1} and R^{2.2} are independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NR^{2C}C(O)NR^{2A}R^{2B}, —N(O)ₘ₂, —NR^{2A}R^{2B}, —C(O)R^{2C}, —C(O) OR^{2C}, —OC(O)R^{2C}, —OC(O) OR^{2C}, —C(O)NR^{2A}R^{2B}, —OC(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, or substituted or unsubstituted heteroalkyl;
R^{1A}, R^{1B}, R^{1C}, R^{2A}, R^{2B}, R^{2C}, and R^{2D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
each X² is independently —F, —Cl, —Br, or —I;
n2 is an integer from 0 to 4; and
m2 and v2 are independently 1 or 2;
wherein at least one of R^{1.1} and R^{1.2} is not hydrogen; and
wherein the compound is not:

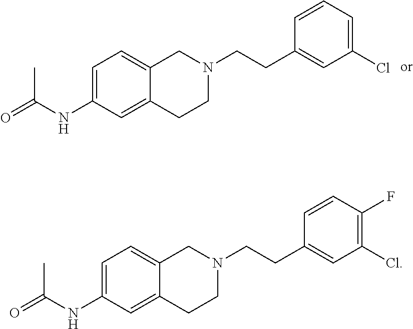 [SMILES: CC(=O)Nc1ccc2c(c1)CCN(CCc1cccc(Cl)c1)C2 ; "or" ; CC(=O)Nc1ccc2c(c1)CCN(CCc1ccc(F)c(Cl)c1)C2]

2. The compound of claim 1, wherein R^{1.1} is halogen, —C(O)NR^{1A}R^{1B}, or —NR^{1A}C(O)R^{1C}; and R^{1.2} is hydrogen.

3. The compound of claim 2, wherein R^{1A} and R^{1B} are independently hydrogen or unsubstituted C₁-C₄ alkyl, and R^{1C} is unsubstituted C₁-C₄ alkyl.

4. The compound of claim 1, wherein R^{1.1} is —NHC(O) CH₃; and R^{1.2} is hydrogen.

5. The compound of claim 1, wherein R^{1.1} is —C(O)NHCH₃; and R^{1.2} is hydrogen.

6. The compound of claim 1, wherein R^{1.1} is hydrogen; and R^{1.2} is halogen, —C(O)NR^{1A}R^{1B}, or —NR^{1A}C(O)R^{1C}.

7. The compound of claim 6, wherein R^{1A} and R^{1B} are independently hydrogen or unsubstituted C₁-C₄ alkyl, and R^{1C} is unsubstituted C₁-C₄ alkyl.

8. The compound of claim 1, wherein R^{1.1} is hydrogen; and R^{1.2} is —NHC(O) CH₃.

9. The compound of claim 1, wherein R^{2.1} and R^{2.2} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, or substituted or unsubstituted heteroalkyl.

10. The compound of claim 1, wherein R^{2.1} and R^{2.2} are independently hydrogen or halogen.

11. The compound of claim 1, wherein R^{2.1} and R^{2.2} are independently hydrogen, —Cl, or —F.

12. The compound of claim 1, wherein n is 0.

13. The compound of claim 1, wherein n is 1.

14. The compound of claim 1, having the formula:

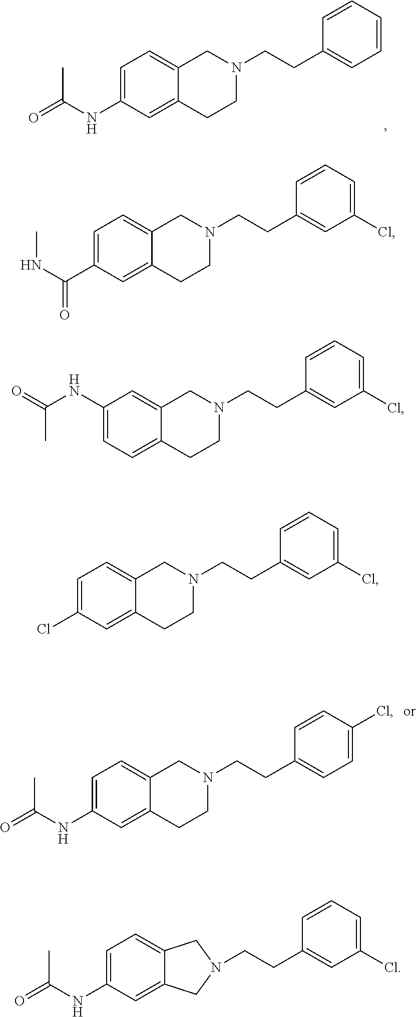 [SMILES: CC(=O)Nc1ccc2c(c1)CCN(CCc1ccccc1)C2 ; CNC(=O)c1ccc2c(c1)CCN(CCc1cccc(Cl)c1)C2 ; CC(=O)Nc1ccc2c(c1)CN(CCc1cccc(Cl)c1)CC2 ; Clc1cccc(CCN2CCc3cc(Cl)ccc3C2)c1 ; CC(=O)Nc1ccc2c(c1)CCN(CCc1ccc(Cl)cc1)C2 ; CC(=O)Nc1ccc2c(c1)CN(CCc1cccc(Cl)c1)C2]

15. A compound, or a pharmaceutically acceptable salt thereof, having the formula: 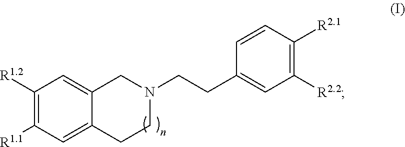
wherein
n is 0 or 1;
R^{1.1} and R^{1.2} are independently hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O) OR^{1C}, —C(O)NR^{1A}R^{1B}, —OC(O)NR^{1A}R^{1B}, —OR^{1D}, —SR 1D, —NR^{1A}SO₂R^{1D}, —NR^{1A}C(O)R^{1C}, —NR^{1A}C(O) OR^{1C}, —NR^{1A}OR^{1C}, —SF₅, —N₃, substituted or unsubstituted alkyl, or substituted or unsubstituted heteroalkyl;
R^{2.1} and R^{2.2} are independently hydrogen or halogen;
R^{1A}, R^{1B}, R^{1C}, and R^{1D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
each X¹ is independently —F, —Cl, —Br, or —I;
n1 is an integer from 0 to 4; and
m1 and v1 are independently 1 or 2;
wherein at least one of R^{2.1} and R^{2.2} is not hydrogen; and
wherein the compound is not:

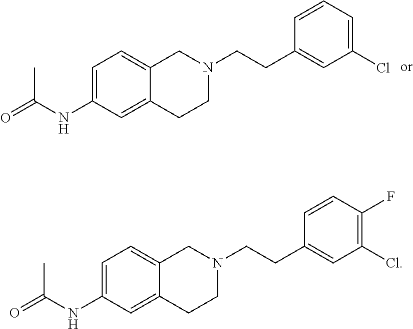 [SMILES: CC(=O)Nc1ccc2c(c1)CCN(CCc1cccc(Cl)c1)C2 ; "or" ; CC(=O)Nc1ccc2c(c1)CCN(CCc1ccc(F)c(Cl)c1)C2]

16. The compound of claim 15, wherein R^{2.1} is hydrogen; and R^{2.2} is halogen.

17. The compound of claim 15, wherein R^{2.1} is hydrogen; and R^{2.2} is —Cl.

18. The compound of claim 15, wherein R^{2.1} is halogen; and R^{2.2} is hydrogen.

19. The compound of claim 15, wherein R^{2.1} is —Cl; and R^{2.2} is hydrogen.

20. The compound of claim 15, wherein R^{2.1} and R^{2.2} are halogen.

21. The compound of claim 15, wherein R^{2.1} is —F; and R^{2.2} is —Cl.

22. The compound of claim 15, wherein R^{1.1} and R^{1.2} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, or substituted or unsubstituted heteroalkyl.

23. The compound of claim 15, wherein R^{1.1} and R^{1.2} are independently hydrogen or —OR^{1D}.

24. The compound of claim 23, wherein R^{1D} is independently hydrogen or unsubstituted C₁-C₄ alkyl.

25. The compound of claim 15, wherein R^{1.1} and R^{1.2} are independently hydrogen or —OCH₃.

26. The compound of claim 15, wherein n is 0.

27. The compound of claim 15, wherein n is 1.

28. The compound of claim 15, having the formula:

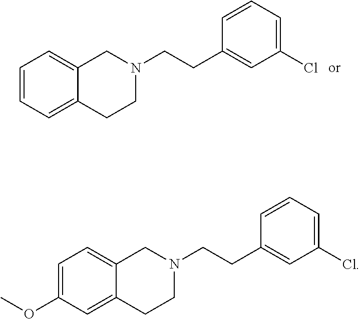 [SMILES: Clc1cccc(CCN2CCc3ccccc3C2)c1 ; COc1ccc2c(c1)CCN(CCc1cccc(Cl)c1)C2]

29. A pharmaceutical composition comprising a pharmaceutically acceptable excipient and the compound of one of claims 1 to 28, or a pharmaceutically acceptable salt thereof, or a compound having the formula: 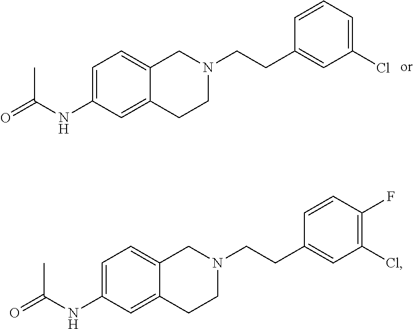 [SMILES: CC(=O)Nc1ccc2c(c1)CCN(CCc1cccc(Cl)c1)C2 ; "or" ; CC(=O)Nc1ccc2c(c1)CCN(CCc1ccc(F)c(Cl)c1)C2]
or a pharmaceutically acceptable salt thereof.

30. The pharmaceutical composition of claim 29, wherein the pharmaceutical composition is an oral dosage form, an intranasal dosage form, an intravenous dosage form, or an injectable dosage form.

31. A method of treating a protein aggregation disease in a subject in need thereof, said method comprising administering to the subject a therapeutically effective amount of the compound of one of claims 1 to 28, or a pharmaceutically acceptable salt thereof, or a compound having the formula: 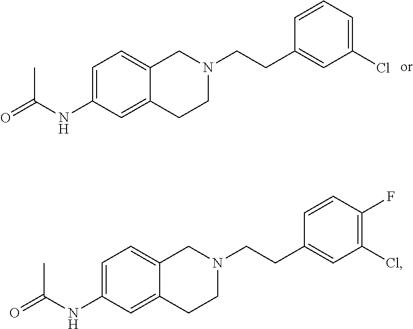 [SMILES: CC(=O)Nc1ccc2c(c1)CCN(CCc1cccc(Cl)c1)C2 ; "or" ; CC(=O)Nc1ccc2c(c1)CCN(CCc1ccc(F)c(Cl)c1)C2]
or a pharmaceutically acceptable salt thereof.

32. The method of claim 31, wherein the protein aggregation disease is a neurodegenerative disease.

33. The method of claim 32, wherein the neurodegenerative disease is frontotemporal dementia, a synucleinopathy, or a tauopathy.

34. The method of claim 32, wherein the neurodegenerative disease is sporadic amyotrophic lateral sclerosis, amyotrophic lateral sclerosis and frontotemporal lobar degeneration, frontotemporal lobar degeneration, Pick's disease, frontotemporal lobar degeneration TDP-43 predominant pathology, frontotemporal lobar degeneration tau predominant pathology, semantic dementia, primary progressive aphasia, multiple system proteinopathy, Perry syndrome, facial onset sensory and motor neuropathy, sporadic inclusion body myositis, myotonic disorder, Alzheimer's disease, Parkinson's disease, dementia with Lewy bodies, Parkinson's disease-associated dementia, multiple system atrophy, corticobasal degeneration, progressive supranuclear palsy, Huntington's disease, C9orf72 amyotrophic lateral sclerosis, amyloidosis, antibody light chain amyloidosis, transthyretin amyloidosis, prion disease, diffuse Lewy body disease, spinocerebellar ataxia, poly-Q disease, hereditary cerebral amyloid angiopathy, or familial amyloid polyneuropathy.

35. The method of claim 34, wherein the prion disease is Creutzfeldt-Jakob disease, variant Creutzfeldt-Jakob disease, genetic human prion disease, bovine spongiform encephalopathy, or scrapie.

36. The method of claim 31, wherein the protein aggregation disease is diabetes.

37. The method of claim 31, wherein the protein aggregation disease is a p53 mutant cancer.

38. The method of claim 31, wherein the protein aggregation disease is an influenza A virus infection or a herpesvirus infection.

39. The method of claim 38, wherein the herpesvirus is an M45 herpesvirus infection.

40. The method of claim 31, wherein the protein aggregation disease is primary systemic amyloidosis, reactive systemic amyloidosis, type II diabetes, injection-localized amyloidosis, beta-2 microglobulin amyloidosis, hereditary non-neuropathic amyloidosis, or Finnish hereditary systemic amyloidosis.

41. The method of claim 31, wherein the compound is administered orally, intranasally, intravenously, or by injection.

42. A method of reducing formation of a protein aggregate or disrupting a protein aggregate, said method comprising contacting the protein aggregate with an effective amount of the compound of one of claims 1 to 28, or a pharmaceutically acceptable salt thereof, or a compound having the formula: 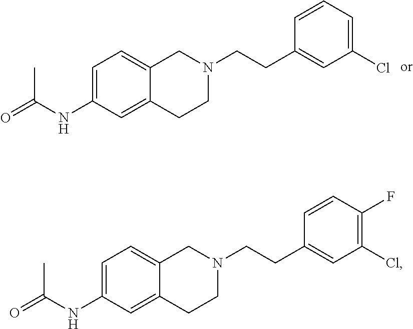 [SMILES: CC(=O)Nc1ccc2c(c1)CCN(CCc1cccc(Cl)c1)C2 ; "or" ; CC(=O)Nc1ccc2c(c1)CCN(CCc1ccc(F)c(Cl)c1)C2]
or a pharmaceutically acceptable salt thereof.

43. The method of claim 42, wherein the protein aggregate is a prion protein aggregate, amyloid precursor protein aggregate, beta amyloid aggregate, alpha synuclein aggregate, superoxide dismutase aggregate, tau aggregate, immunoglobulin aggregate, amyloid-A aggregate, transthyretin aggregate, beta-2 microglobulin aggregate, cystatin C aggregate, apolipoprotein A1 aggregate, TDP-43 aggregate, islet amyloid polypeptide aggregate, ANF aggregate, gelsolin aggregate, insulin aggregate, lysozyme aggregate, fibrinogen aggregate, huntingtin aggregate, or ataxin aggregate.

44. The method of claim 42, wherein the protein aggregate is a beta amyloid aggregate.

45. The method of claim 42, wherein the protein aggregate is a tau aggregate.

46. The method of claim 42, wherein the protein aggregate is an alpha synuclein aggregate.

47. The method of claim 42, wherein the protein aggregate is a TDP-43 aggregate.

48. The method of claim 42, wherein the protein aggregate is an oligomer.

49. The method of claim 42, wherein the contacting occurs in vivo.

50. The method of claim 42, wherein the contacting occurs in a subject, wherein an effective amount of the compound is administered to the subject.

51. The method of claim 50, wherein the subject has a protein aggregation disease.
